(19) United States
(12) Patent Application Publication
(10) Pub. No.: US 2021/0338782 A1
(43) Pub. Date: Nov. 4, 2021

(54) STORAGE STABLE SOMATOSTATIN-DOPAMINE CHIMERIC COMPOUNDS AND SALT FORMS THEREOF

(71) Applicant: Cydan II, Inc., Cambridge, MA (US)

(72) Inventors: Alfredo GROSSI, Stoneham, MA (US); Heather HALEM, Westborough, MA (US); Niels SVENSTRUP, Brookline, MA (US); Valerie CWYNAR, Dracut, MA (US)

(21) Appl. No.: 17/320,760

(22) Filed: May 14, 2021

Related U.S. Application Data
(63) Continuation of application No. PCT/US2020/054182, filed on Oct. 3, 2020.
(60) Provisional application No. 63/007,786, filed on Apr. 9, 2020.
(60) Provisional application No. 62/911,101, filed on Oct. 4, 2019.

(51) Int. Cl. A61K 38/31; A61K 31/4745; A61K 47/64; A61K 47/55; A61K 9/00

(52) U.S. Cl. CPC A61K 38/31; A61K 31/4745; A61K 9/0019; A61K 47/55; A61K 47/64

(57) ABSTRACT

The disclosure provides storage-stable somatostatin-dopamine chimeric analog compounds and storage-stable pharmaceutical compositions thereof for use in treating endocrine diseases and endocrine tumors.

### 1. CROSS-REFERENCE TO RELATED APPLICATIONS

This application is a continuation of PCT/US2020/054182, filed Oct. 3, 2020, which claims the benefit of and priority to U.S. Provisional Application Nos. 62/911,101, filed Oct. 4, 2019; and 63/007,786, filed Apr. 9, 2020. The content of each of the above-referenced applications is incorporated by reference in its entirety.

### 2. BACKGROUND

Non-functioning pituitary adenomas (NFPAs) are non-metastatic tumors of the pituitary gland that can grow to compress cerebral structures, including the optic nerve and carotid artery. NFPAs cause debilitating symptoms in patients, including hypertension, headaches, hypopituitarism, and even loss of vision. The current standard of care for treating NFPAs is trans-sphenoidal surgery, an invasive brain surgery that requires general anesthesia and results in partial retention of the tumor in approximately 50% of treated patients (Chen et al., 2012, Neuroendocrinology 96 (4): 333-42). Approximately 40% of NFPA patients treated with trans-sphenoidal surgery experience tumor regrowth within five years (Chen et al. (2012) and Dekkers et al., 2008, The Journal of Clinical Endocrinology and Metabolism 93 (10): 3717-26). Thus, there exists a need for an improved medical treatment for NFPAs.

Expression of dopamine receptor type 2 (D2R) and somatostatin receptors has been demonstrated in NFPAs, raising the possibility that dopamine agonists and/or somatostatin analogs may be useful medical agents for treating NFPAs (Drummond et al., 2018 Nov. 28, in: Feingold et al., editors, Endotext [Internet]. South Dartmouth (MA): MDText.com, Inc.; 2000-. Available from: https://www.ncbi.nlm.nih.gov/books/NBK534880/). Existing somatostatin-dopamine chimeric compounds are described in U.S. Pat. Nos. 7,517,853 and 8,822,442 as well as US PreGrant publication no. 2011/0065632, each of which is hereby incorporated by reference in its entirety.

There is a need for somatostatin-dopamine chimeric compounds that have improved stability and that lack N-acetyl-lysine as a degradation impurity to provide improved medical therapies for treating NFPAs.

### 3. SUMMARY OF THE INVENTION

TBR-760, previously known as BIM-23A760, is a chimeric somatostatin (SST)-dopamine (DA) compound with potent agonist activity at both SST type 2 and DA type 2 receptors. The compound name is Dop2-D-Lys(Dop2)-cyclo[Cys-Tyr-D-Trp-Lys-Abu-Cys]-Thr-NH₂, where “Dop2” refers to the compound of formula (II):

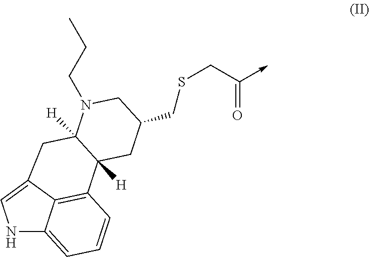

The structure of TBR-760 is shown in FIG. 44B. Both the macrocyclic peptide moiety and the Dop2 cyclic group would be expected to contribute to TBR-760's physicochemical properties.

In prior pre-clinical and clinical studies, TBR-760 was administered as the acetate salt. Acetate is the most commonly used salt form for peptide therapeutics.

We have now discovered that the acetate form of TBR-760 is surprisingly unstable, an instability that had not been identified during preclinical and early clinical development. Moreover, the instability of TBR-760 acetate cannot be predicted from the behavior of the structurally similar somatostatin analog, lanreotide.

Lanreotide is a somatostatin analogue approved by FDA to slow the growth of gastrointestinal and pancreatic neuroendocrine tumors. Lanreotide shares major structural features with TBR-760; the structures are compared in FIGS. 44A and 44B. Lanreotide is approved and sold as the acetate salt.

We have now discovered that while lanreotide acetate is both thermostable and stable to both visible and UV light, TBR-760 acetate is not. In addition, we have found that while lanreotide hydrobromide is moderately stable to heat and humidity and to UV and visible light, the hydrobromide salt of TBR-760 is significantly degraded under these conditions. Surprisingly, however, TBR-760 hydrochloride is thermostable, and the hydrochloride salt is as photostable as TBR-760 acetate.

Accordingly, in a first aspect, the compound, Dop2-D-Lys(Dop2)-cyclo[Cys-Tyr-D-Trp-Lys-Abu-Cys]-Thr-NH₂ (TBR-760) hydrochloride salt, is provided. In typical embodiments, the compound is Dop2-D-Lys(Dop2)-cyclo[Cys-Tyr-D-Trp-Lys-Abu-Cys]-Thr-NH₂(TBR-760) trihydrochloride.

In some embodiments, less than 1% of the chimeric peptide of the pharmaceutical composition is degraded after 8 weeks at 60° C. In some embodiments, less than 0.5% of the chimeric peptide is degraded after 4 weeks at 40° C. In some embodiments, less than 0.8% of the peptide is degraded after 4 weeks at 60° C. In some embodiments, less than 0.1% of the peptide is degraded after 4 weeks at 25° C.

In another aspect, provided herein is a storage stable pharmaceutical composition comprising a pharmaceutically acceptable salt of Dop2-D-Lys(Dop2)-cyclo[Cys-Tyr-D-Trp-Lys-Abu-Cys]-Thr-NH₂, and, optionally, water in an amount of less than 10%. In some embodiments, the salt is hydrochloride. In some embodiments, the salt is trihydrochloride. In some embodiments, the salt is sulfate. In some embodiments, the salt is mesylate. In some embodiments, the salt is tosylate.

In some embodiments, the pharmaceutical composition disclosed herein has a purity profile as shown in the eight week profile of FIG. 2. In some embodiments, the pharmaceutical composition has a purity profile as shown in the eight week profile of FIG. 3. In some embodiments, the pharmaceutical composition has a purity profile as shown in the eight week profile of FIG. 4. In some embodiments, the pharmaceutical composition has a purity profile as shown in the eight week profile of FIG. 5.

In some embodiments, less than 1% of the composition is degraded after 8 weeks at 60° C. In some embodiments, less than 0.8% of the composition is degraded after 4 weeks at 60° C. In some embodiments, less than 0.5% of the composition is degraded after 4 weeks at 40° C. In some embodiments, less than 0.1% of the composition is degraded after 4 weeks at 25° C.

In some embodiments, the degradation products of the pharmaceutical composition disclosed herein do not include N-acetyl-lysine.

In some embodiments, the pharmaceutical composition disclosed herein is in a prefilled syringe. In some embodiments, the prefilled syringe contains a single dose of the pharmaceutical composition. In some embodiments, the prefilled syringe is in an automated injection device.

In another aspect, provided herein is a method of treating a subject having an endocrine disease or an endocrine tumor, comprising administering to the subject an effective amount of the pharmaceutical composition. In some embodiments, the endocrine tumor is a neuroendocrine tumor. In some embodiments, the neuroendocrine tumor is a non-functioning pituitary adenoma (NFPA).

In some embodiments, the administering of the pharmaceutical composition is in an amount of 0.5 mg to 10 mg per week.

In another aspect, provided herein is a kit comprising the pharmaceutical composition disclosed herein and a diluent. In some embodiments, the kit further comprises an injection syringe, a vial comprising the pharmaceutical composition as a lyophilate, a vial comprising the diluent, and a transfer syringe. In some embodiments, the diluent comprises water, trehalose, and pH adjusters.

### 4. BRIEF DESCRIPTION OF THE SEVERAL VIEWS OF THE DRAWINGS

These and other features, aspects, and advantages of the present invention will become better understood with regard to the following description, and accompanying drawings, where:

FIG. 1 shows results of thermostability studies of the TBR-760 acetate salt over 2, 4, and 8 weeks.

FIG. 2 shows results of thermostability studies of the TBR-760 hydrochloride salt over 2, 4, and 8 weeks.

FIG. 3 shows results of thermostability studies of the TBR-760 sulfate salt over 2, 4, and 8 weeks.

FIG. 4 shows results of thermostability studies of the TBR-760 mesylate salt over 2, 4, and 8 weeks.

FIG. 5 shows results of thermostability studies of the TBR-760 tosylate salt over 2, 4, and 8 weeks.

FIG. 6 shows results of pharmacokinetic studies of TBR-760 salts in rat plasma: plasma concentration over time of TBR-760 following administration of each salt form is shown in FIG. 6A; maximum plasma concentration and time to maximum concentration for each salt form are shown in FIG. 6B.

FIG. 7 shows Draize Scoring results of injection sites of minipigs administered various TBR-760 salt forms over 7 days.

FIG. 8 shows thermogravimetric analysis (TGA) curves of all five TBR-760 salts.

FIG. 9 shows differential scanning calorimetry (DSC) traces of all five TBR-760 salts.

FIGS. 10A, 10B, and 10C graphically represent the drug purity of all five TBR-760 salts following exposure to various conditions. FIG. 10A shows results of salts exposed to 25° C. and 60% relative humidity (RH); FIG. 10B shows results of salts exposed to 40° C. and 75% RH;

FIG. 10C shows results of salts exposed to 60° C. in open dishes.

FIG. 11A shows a chromatogram generated by UPLC of TBR-760 acetate exposed to control conditions. FIG. 11B shows a chromatogram generated by UPLC of TBR-760 acetate exposed to 60° C. in an open dish for two weeks.

FIG. 12A shows a chromatogram generated by UPLC of TBR-760 chloride exposed to control conditions. FIG. 12B shows a chromatogram generated by UPLC of TBR-760 chloride exposed to 60° C. in an open dish for two weeks.

FIG. 13A shows a chromatogram generated by UPLC of TBR-760 sulfate exposed to control conditions. FIG. 13B shows a chromatogram generated by UPLC of TBR-760 sulfate exposed to 60° C. in an open dish for two weeks.

FIG. 14A shows a chromatogram generated by UPLC of TBR-760 mesylate exposed to control conditions. FIG. 14B shows a chromatogram generated by UPLC of TBR-760 mesylate exposed to 60° C. in an open dish for two weeks.

FIG. 15A shows a chromatogram generated by UPLC of TBR-760 mesylate exposed to control conditions. FIG. 15B shows a chromatogram generated by UPLC of TBR-760 mesylate exposed to 60° C. in an open dish for two weeks.

FIG. 16A shows drug purity results of TBR-760 acetate, chloride, mesylate, sulfate, and tosylate salts exposed to visible light during photostability studies. FIG. 16B shows drug purity results of TBR-760 acetate, chloride, mesylate, sulfate, and tosylate salts exposed to UV light during photostability studies.

FIG. 17 is a photograph showing samples of TBR-760 acetate, chloride, mesylate, sulfate, and tosylate salts following exposure to visible light during photostability studies.

FIG. 18 is a photograph showing samples of TBR-760 acetate, chloride, mesylate, sulfate, and tosylate salts following exposure to UV light during photostability studies.

FIGS. 19A and 19B show results of UPLC analysis of TBR-760 acetate after being stored in sealed, amber vials at −20° C. Undegraded TBR-760 is indicated with an arrow in FIG. 19B.

FIGS. 20A and 20B show results of UPLC analysis of TBR-760 acetate incubated in a visible light chamber while contained in a sealed, amber vial wrapped in aluminum foil. Undegraded TBR-760 is indicated with an arrow in FIG. 20B.

FIGS. 21A and 21B show results of UPLC analysis of TBR-760 acetate incubated in a UV light chamber while contained in a sealed, amber vial wrapped in aluminum foil. Undegraded TBR-760 is indicated with an arrow in FIG. 21B.

FIG. 22A shows a chromatogram of TBR-760 acetate following exposure to visible light while contained in a sealed, clear vial. FIG. 22B shows a chromatogram of TBR-760 acetate following exposure to UV light while contained in a sealed, clear vial.

FIG. 23A shows purity results for TBR 760 acetate following exposure to visible light. FIG. 23B shows purity results for TBR 760 acetate following exposure to UV light. Undegraded TBR-760 is indicated with arrows in FIG. 23A and FIG. 23B.

FIGS. 24A and 24B show results of UPLC analysis of TBR-760 chloride after being stored in sealed, amber vials at −20° C. Undegraded TBR-760 is indicated with an arrow in FIG. 24B.

FIGS. 25A and 25B show results of UPLC analysis of TBR-760 chloride incubated in a visible light chamber while contained in a sealed, amber vial wrapped in aluminum foil. Undegraded TBR-760 is indicated with an arrow in FIG. 25B.

FIGS. 26A and 26B show results of UPLC analysis of TBR-760 chloride incubated in a UV light chamber while contained in a sealed, amber vial wrapped in aluminum foil. Undegraded TBR-760 is indicated with an arrow in FIG. 26B.

FIG. 27A shows a chromatogram of TBR-760 chloride following exposure to visible light while contained in a sealed, clear vial. FIG. 27B shows a chromatogram of TBR-760 chloride following exposure to UV light while contained in a sealed, clear vial.

FIG. 28A shows purity results for TBR 760 chloride following exposure to visible light. FIG. 28B shows purity results for TBR 760 chloride following exposure to UV light. Undegraded TBR-760 is indicated with arrows in FIG. 28A and FIG. 28B.

FIGS. 29A and 29B show results of UPLC analysis of TBR-760 mesylate after being stored in sealed, amber vials at −20° C. Undegraded TBR-760 is indicated with an arrow in FIG. 29B.

FIGS. 30A and 30B show results of UPLC analysis of TBR-760 mesylate incubated in a visible light chamber while contained in a sealed, amber vial wrapped in aluminum foil. Undegraded TBR-760 is indicated with an arrow in FIG. 30B.

FIGS. 31A and 31B show results of UPLC analysis of TBR-760 mesylate incubated in a UV light chamber while contained in a sealed, amber vial wrapped in aluminum foil. Undegraded TBR-760 is indicated with an arrow in FIG. 31B.

FIG. 32A shows a chromatogram of TBR-760 mesylate following exposure to visible light while contained in a sealed, clear vial. FIG. 32B shows a chromatogram of TBR-760 mesylate following exposure to UV light while contained in a sealed, clear vial.

FIG. 33A shows purity results for TBR-760 mesylate following exposure to visible light. FIG. 33B shows purity results for TBR-760 mesylate following exposure to UV light. Undegraded TBR-760 is indicated with arrows.

FIGS. 34A and 34B show results of UPLC analysis of TBR-760 sulfate after being stored in sealed, amber vials at −20° C. Undegraded TBR-760 is indicated with an arrow in FIG. 34B.

FIGS. 35A and 35B show results of UPLC analysis of TBR-760 sulfate incubated in a visible light chamber while contained in a sealed, amber vial wrapped in aluminum foil. Undegraded TBR-760 is indicated with an arrow in FIG. 35B.

FIGS. 36A and 36B show results of UPLC analysis of TBR-760 sulfate incubated in a UV light chamber while contained in a sealed, amber vial wrapped in aluminum foil. Undegraded TBR-760 is indicated with an arrow in FIG. 36B.

FIG. 37A shows a chromatogram of TBR-760 sulfate following exposure to visible light while contained in a sealed, clear vial. FIG. 37B shows a chromatogram of TBR-760 sulfate following exposure to UV light while contained in a sealed, clear vial.

FIG. 38A shows purity results for TBR 760 sulfate following exposure to visible light. FIG. 38B shows purity results for TBR 760 sulfate following exposure to UV light. Undegraded TBR-760 is indicated with arrows.

FIGS. 39A and 39B show results of UPLC analysis of TBR-760 tosylate after being stored in sealed, amber vials at −20° C. Undegraded TBR-760 is indicated with an arrow in FIG. 39B.

FIGS. 40A and 40B show results of UPLC analysis of TBR-760 tosylate incubated in a visible light chamber while contained in a sealed, amber vial wrapped in aluminum foil. Undegraded TBR-760 is indicated with an arrow in FIG. 40B.

FIGS. 41A and 41B show results of UPLC analysis of TBR-760 tosylate incubated in a UV light chamber while contained in a sealed, amber vial wrapped in aluminum foil. Undegraded TBR-760 is indicated with an arrow in FIG. 41B.

FIG. 42A shows a chromatogram of TBR-760 tosylate following exposure to visible light while contained in a sealed, clear vial. FIG. 42B shows a chromatogram of TBR-760 tosylate following exposure to UV light while contained in a sealed, clear vial.

FIG. 43A shows purity results for TBR-760 tosylate following exposure to visible light. FIG. 43B shows purity results for TBR-760 sulfate following exposure to UV light. Undegraded TBR-760 is indicated with arrows in each of FIGS. 43A and 43B.

FIG. 44A shows the chemical structure of lanreotide. FIG. 44B shows the chemical structure of TBR-760.

FIG. 45 shows purity results of lanreotide acetate and TBR-760 acetate following exposure to 25° C. at 60% relative humidity (RH); 40° C. at 75% RH; or 60° C. at ambient humidity for 0, 2, or 4 weeks.

FIG. 46 shows purity results of lanreotide bromide and TBR-760 bromide following exposure to 25° C. at 60% relative humidity (RH); 40° C. at 75% RH; or 60° C. at ambient humidity for 0, 2, or 4 weeks.

FIG. 47 shows purity results of lanreotide chloride and TBR-760 chloride following exposure to 25° C. at 60% relative humidity (RH); 40° C. at 75% RH; or 60° C. at ambient humidity for 0, 2, or 4 weeks.

FIG. 48A and FIG. 48B summarize the impurity analysis of lanreotide acetate (FIG. 48A) and TBR-760 acetate (FIG. 48B) following exposure of each compound to conditions of 25° C. at 60% relative humidity (RH); 40° C. at 75% RH; or 60° C. at ambient humidity for 0, 2, or 4 weeks. The calculated percent purity of the subject compound in each sample is reported on each bar in the graph. The number of impurities in each sample is shown above the bar representing each sample.

FIG. 49A and FIG. 49B summarize the impurity analysis of lanreotide bromide (FIG. 49A) and TBR-760 bromide (FIG. 49B) following exposure of each compound to conditions of 25° C. at 60% relative humidity (RH); 40° C. at 75% RH; or 60° C. at ambient humidity for 0, 2, or 4 weeks. The calculated percent purity of the subject compound in each sample is reported on each bar in the graph. The number of impurities in each sample is shown above the bar representing each sample.

FIG. 50A and FIG. 50B summarize the impurity analysis of lanreotide chloride (FIG. 50A) and TBR-760 chloride (FIG. 50B) following exposure of each compound to conditions of 25° C. at 60% relative humidity (RH); 40° C. at 75% RH; or 60° C. at ambient humidity for 0, 2, or 4 weeks. The calculated percent purity of the subject compound in each sample is reported on each bar in the graph. The number of impurities in each sample is shown above the bar representing each sample.

FIGS. 51A and 51B are photographs of lanreotide acetate (FIG. 51A) and TBR-760 acetate (FIG. 51B) following exposure to either 1.2 million lux hours of visible light or 200 watt hours/square meter of UV light.

FIG. 52A and FIG. 52B are photographs of lanreotide bromide (FIG. 52A) and TBR-760 bromide (FIG. 52B) following exposure to either 1.2 million lux hours of visible light or 200 watt hours/square meter of UV light.

FIGS. 53A and 53B are images of lanreotide chloride (FIG. 53A) and TBR-760 chloride (FIG. 53B) following exposure to either 1.2 million lux hours of visible light or 200 watt hours/square meter of UV light.

FIG. 54 shows purity results of lanreotide acetate and TBR-760 acetate following exposure to visible and UV light.

FIG. 55 shows purity results of lanreotide bromide and TBR-760 bromide following exposure to visible and UV light.

FIG. 56 shows purity results of lanreotide chloride and TBR-760 chloride following exposure to visible and UV light.

FIG. 57 compares decreases in purity of lanreotide and TBR-760 salts following exposure to visible and UV light.

FIGS. 58A and 58B summarize the impurity analysis of lanreotide salts (FIG. 58A) and TBR-760 salts (FIG. 58B) following exposure of each compound to 200 watt hours/square meter of UV light. The calculated percent purity of the subject compound in each sample is reported on each bar in the graph. The number of impurities in each sample is shown above the bar representing each sample.

FIGS. 59A and 59B summarize the impurity analysis of lanreotide salts (FIG. 58A) and TBR-760 salts (FIG. 58B) following exposure of each compound to 1.2 million lux hours of visible light. The calculated percent purity of the subject compound in each sample is reported on each bar in the graph. The number of impurities in each sample is shown above the bar representing each sample

### 5. DETAILED DESCRIPTION OF THE INVENTION

We have discovered that the acetate salt form of TBR-760 is surprisingly unstable, an instability that had not been identified during preclinical and early clinical development and that cannot be predicted from the behavior of the structurally similar somatostatin analog, lanreotide. We have further discovered that certain other salt forms of TBR-760 have increased temperature stability with sufficient photostability, to allow TBR-760 to be stored, shipped, and maintained at the point of use for increased periods of time without degrading, and further allowing TBR-760 to be packaged in prefilled syringes and autoinject pens for use by patients with various endocrine tumors, including but not limited to non-functioning pituitary adenomas (NFPA).

### 5.1. Definitions

Unless otherwise defined herein, all technical and scientific terms used herein have the meaning commonly understood by one of ordinary skill in the art to which the invention pertains.

“TBR-760” (formerly known as BIM-23A760) refers the compound of formula (I):

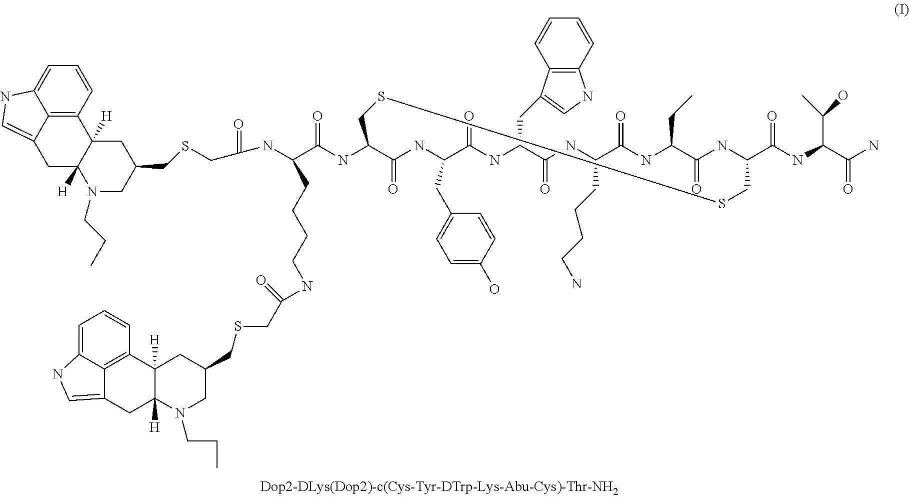 [SMILES: CCCN1C[C@H](CSCC(=O)NCCCC[C@@H](NC(=O)CSC[C@@H]2C[C@@H]3c4cccc5[nH]cc(c45)C[C@H]3N(CCC)C2)C(=O)N[C@H]2CSSC[C@@H](C(=O)N[C@H](C(N)=O)[C@@H](C)O)NC(=O)[C@H](CC)NC(=O)[C@H](CCCCN)NC(=O)[C@@H](Cc3c[nH]c4ccccc34)NC(=O)[C@H](Cc3ccc(O)cc3)NC2=O)C[C@@H]2c3cccc4[nH]cc(c34)C[C@H]21]

As used herein, “Dop2” refers to the compound of formula (II):

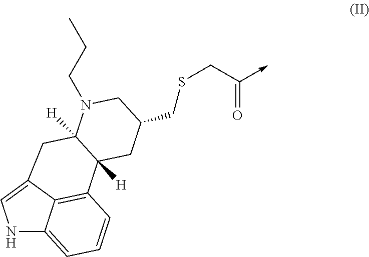

As used herein, the abbreviation “TFA” refers to trifluoro acetic acid.

As used herein, “somatostatin agonists” are those compounds that bind to at least one somatostatin receptor (SSTR), including but not limited to SSTR-1, SSTR-2, SSTR-3, SSTR-4, and SSTR-5, and that upon binding act as an agonist at the SSTR.

“Dopamine agonists” are those compounds that bind to at least one dopamine receptor, including but not limited to D1, D2, D3, D4, and D5 dopamine receptors, and that upon binding act as an agonist at the dopamine receptor.

As used herein, the term “subject” broadly refers to any animal, including but not limited to, human and non-human animals (e.g., dogs, cats, cows, horses, sheep, pigs, poultry, fish, crustaceans, etc.). “Patient” is used synonymously with “human subject.”

As used herein, the term “effective amount” refers to the amount of a composition (e.g., a synthetic peptide) sufficient to effect one or more beneficial or desired results. An effective amount can be administered in one or more administrations, applications or dosages and is not intended to be limited to a particular formulation or administration route.

The term “therapeutically effective amount” is an amount that is effective to achieve a medically desirable therapeutic goal; therapeutically effective amounts need not be curative.

A “prophylactically effective amount” is an amount that is effective to prevent one or more signs or symptoms of a disease, including but not limited to progression in tumor size.

It must be noted that, as used in the specification and the appended claims, the singular forms “a,” “an” and “the” include plural referents unless the context clearly dictates otherwise.

### 5.2. Storage-Stable Somatostatin-Dopamine Chimeric Analogs

In a first aspect, storage-stable salts of somatostatin-dopamine chimeric analogs are provided.

The somatostatin-dopamine chimeric analog comprises at least one moiety that binds to one or more somatostatin receptors (a somatostatin agonist) and at least one moiety that binds to one or more dopamine receptors (a dopamine agonist). In some embodiments, the chimeric analog binds to the SSTR2 receptor. In some embodiments, the chimeric analog binds to the SSTR5 receptor. In some embodiments, the chimeric analog binds to the D2 receptor. In some embodiments, the chimeric analog binds to the SSTR2, SSTR5, and D2 receptors.

In various embodiments, the somatostatin-dopamine chimeric analog is a compound described in U.S. Pat. Nos. 7,517,853, 8,822,442, and/or US Pre-grant Pub. No. 2011/0065632, each of which is incorporated herein by reference in its entirety, or a stereoisomer, hydrate, solvate, deuterated analog or fluorinated analog thereof.

In some embodiments, the salt of the somatostatin-dopamine chimeric analog is the hydrochloride salt. In some embodiments, the salt of the somatostatin-dopamine chimeric analog is the sulfate salt. In some embodiments, the salt of the somatostatin-dopamine chimeric analog is the mesylate salt. In some embodiments, the salt of the somatostatin-dopamine chimeric analog is the tosylate salt.

In particular embodiments, the somatostatin-dopamine chimeric analog is TBR-760, or a stereoisomer, hydrate, solvate, deuterated analog or fluorinated analog thereof. In certain currently preferred embodiments, the storage-stable salt is the chloride salt of TBR-760: Dop2-D-Lys(Dop2)-cyclo[Cys-Tyr-D-Trp-Lys-Abu-Cys]-Thr-NH₂ hydrochloride salt. In particular embodiments, storage-stable salt is the trihydrochloride salt of TBR-760: Dop2-D-Lys(Dop2)-cyclo[Cys-Tyr-D-Trp-Lys-Abu-Cys]-Thr-NH₂ trihydrochloride.

### 5.3. Storage-Stable Pharmaceutical Compositions of Somatostatin-Dopamine Chimeric Analogs

In another aspect, storage stable pharmaceutical compositions are provided, the compositions comprising at least one storage-stable salt of a somatostatin-dopamine chimeric analog as described in Section 5.2, or a stereoisomer, hydrate, solvate, deuterated analog or fluorinated analog thereof, as an active pharmaceutical ingredient (API), and, optionally, water. In particular embodiments, the pharmaceutical composition comprises water in an amount of less than 10%, 9%, 8%, 7%, 6%, or 5% (w/w).

In some embodiments, the pharmaceutical composition further comprises at least one pharmaceutically acceptable diluent, buffer, excipient, carrier, or stabilizer.

In various embodiments, the pharmaceutical composition is formulated for intravenous administration, intrathecal administration, intra-cisterna magna administration, intraventricular administration, subcutaneous administration, intramuscular administration, or intraperitoneal administration. In certain embodiments, the pharmaceutical composition further comprises at least one pharmaceutically acceptable diluent, buffer, excipient, carrier, or stabilizer suitable respectively for intravenous administration, intrathecal administration, intra-cisterna magna administration, intraventricular administration, subcutaneous administration, intramuscular administration, or intraperitoneal administration.

In some embodiments, the pharmaceutical composition comprises at least one preservative, stabilizer, buffer, or antioxidant. In some embodiments, the pharmaceutical composition comprises trehalose. In some embodiments, the pharmaceutical composition comprises an organic or inorganic salt such as, but not limited to, a hydrochloride salt, sulfate salt, mesylate salt, acetate salt, or tosylate salt.

In certain embodiments, the pharmaceutical composition is a parenterally acceptable aqueous solution which is pyrogen-free and has suitable pH, isotonicity and stability. Those of skill in the art are well able to prepare suitable solutions using, for example, isotonic vehicles such as Sodium Chloride Injection, Ringer's Injection, Lactated Ringer's Injection.

In various embodiments, at least one of the at least one active pharmaceutical ingredient is a storage-stable salt of TBR-760, or a stereoisomer, hydrate, solvate, deuterated analog or fluorinated analog thereof. In currently preferred embodiments, the storage-stable salt is the chloride salt of TBR-760: Dop2-D-Lys(Dop2)-cyclo[Cys-Tyr-D-Trp-Lys-Abu-Cys]-Thr-NH₂ hydrochloride salt. In particular embodiments, the storage-stable salt is the trihydrochloride salt of TBR-760: Dop2-D-Lys(Dop2)-cyclo[Cys-Tyr-D-Trp-Lys-Abu-Cys]-Thr-NH₂ trihydrochloride.

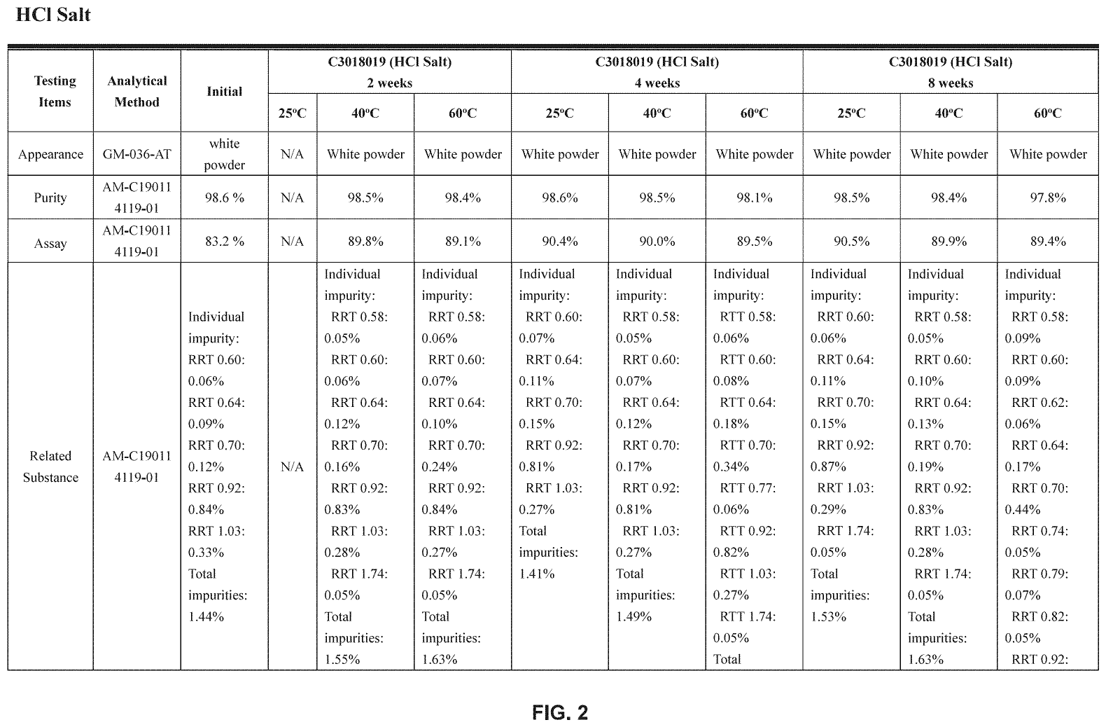

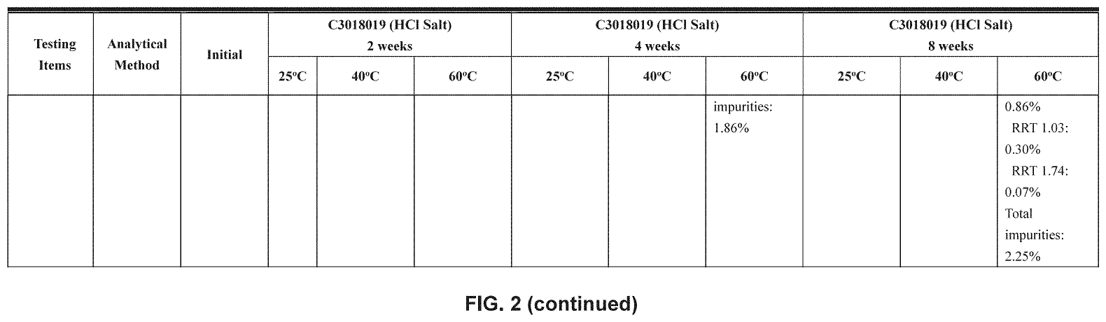

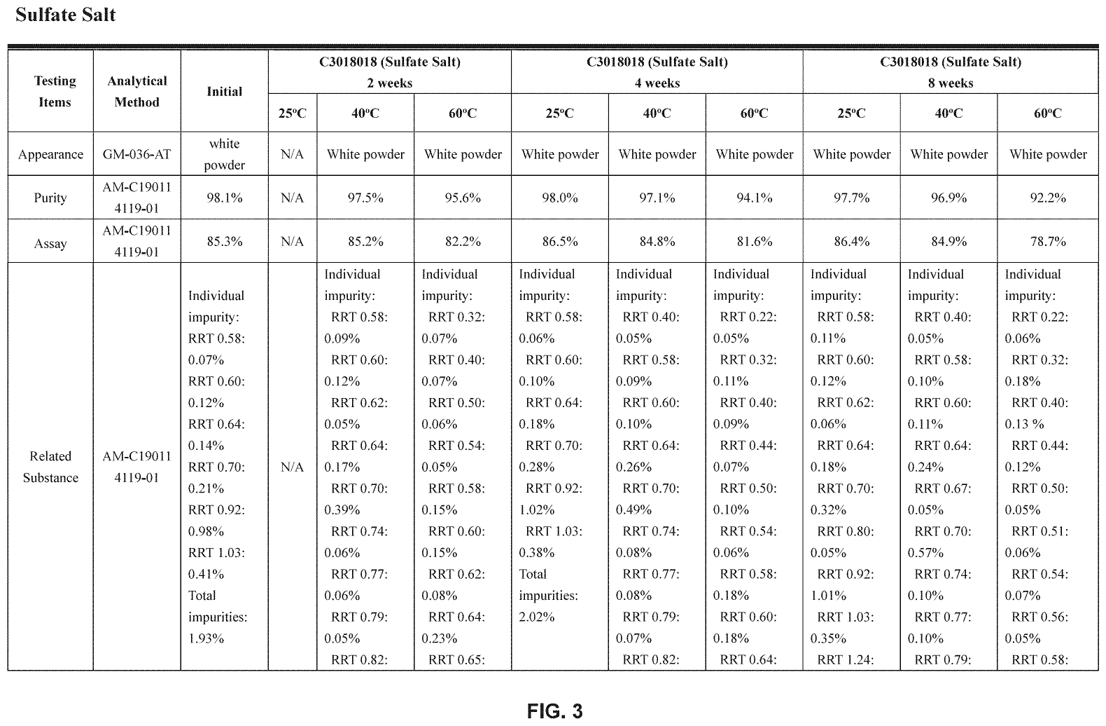

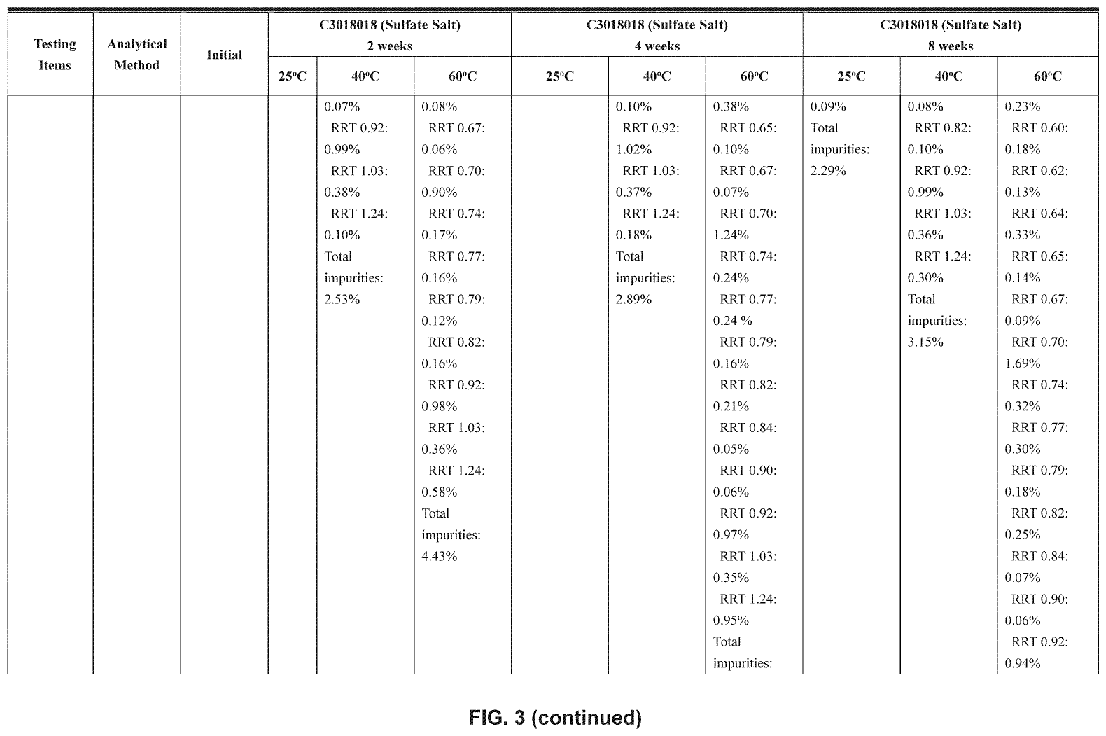

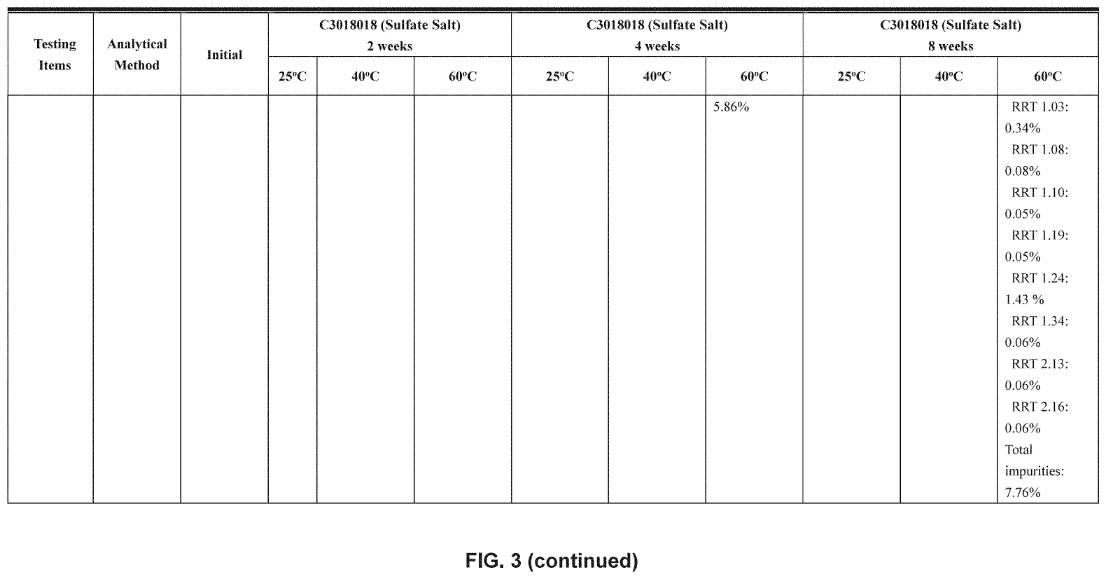

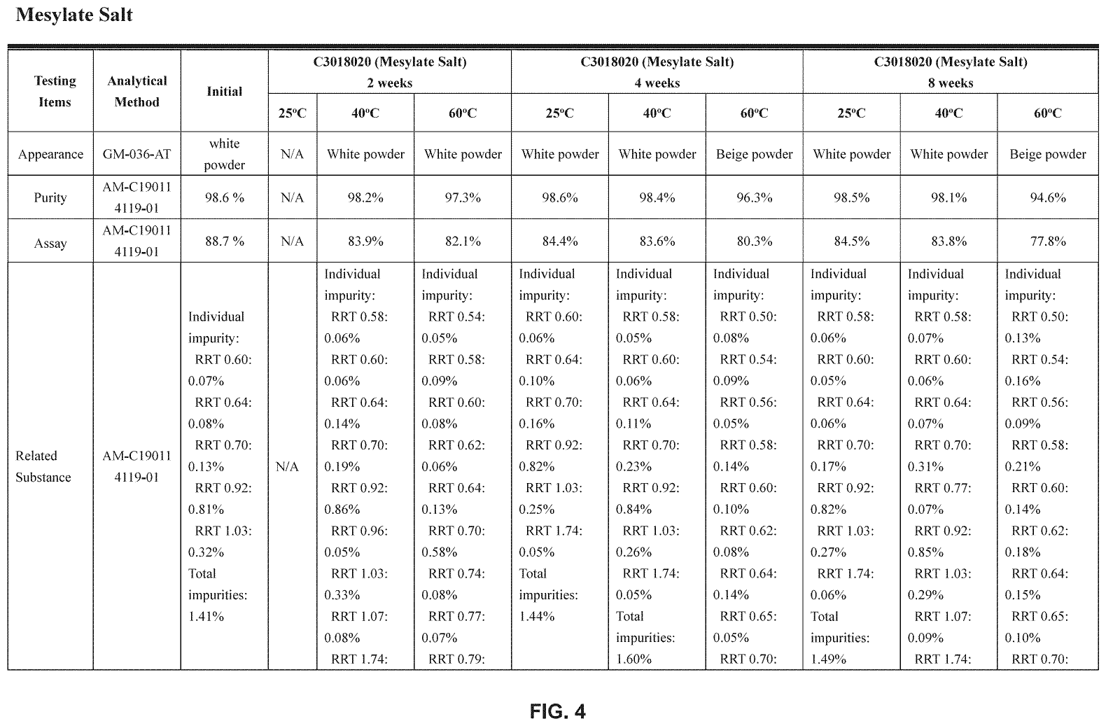

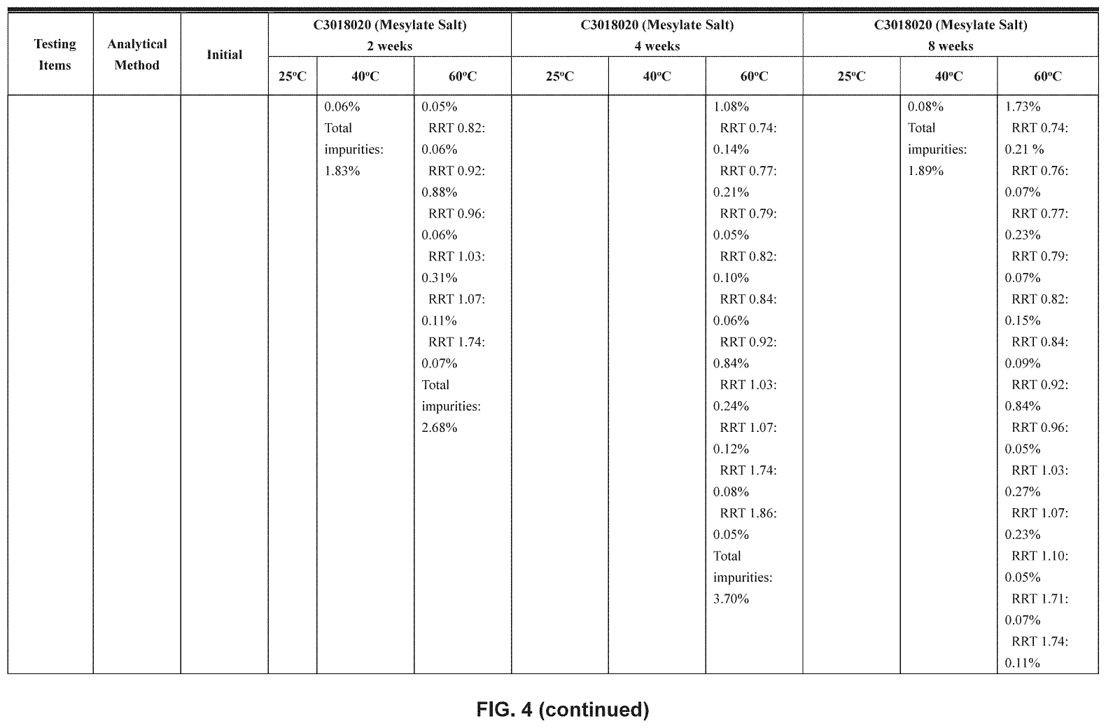

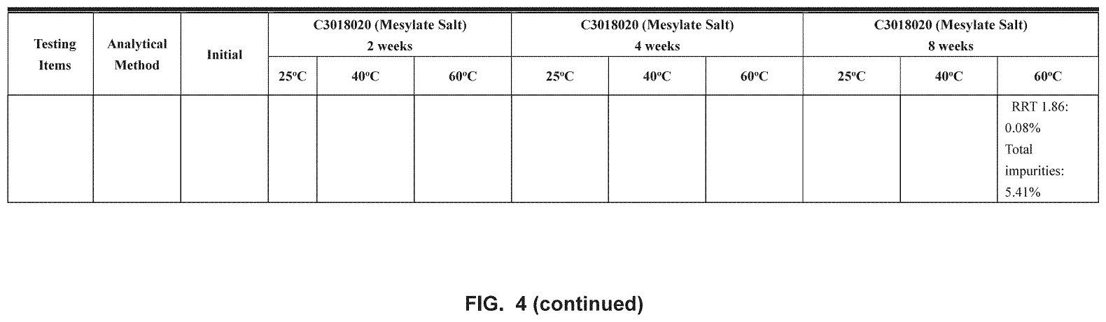

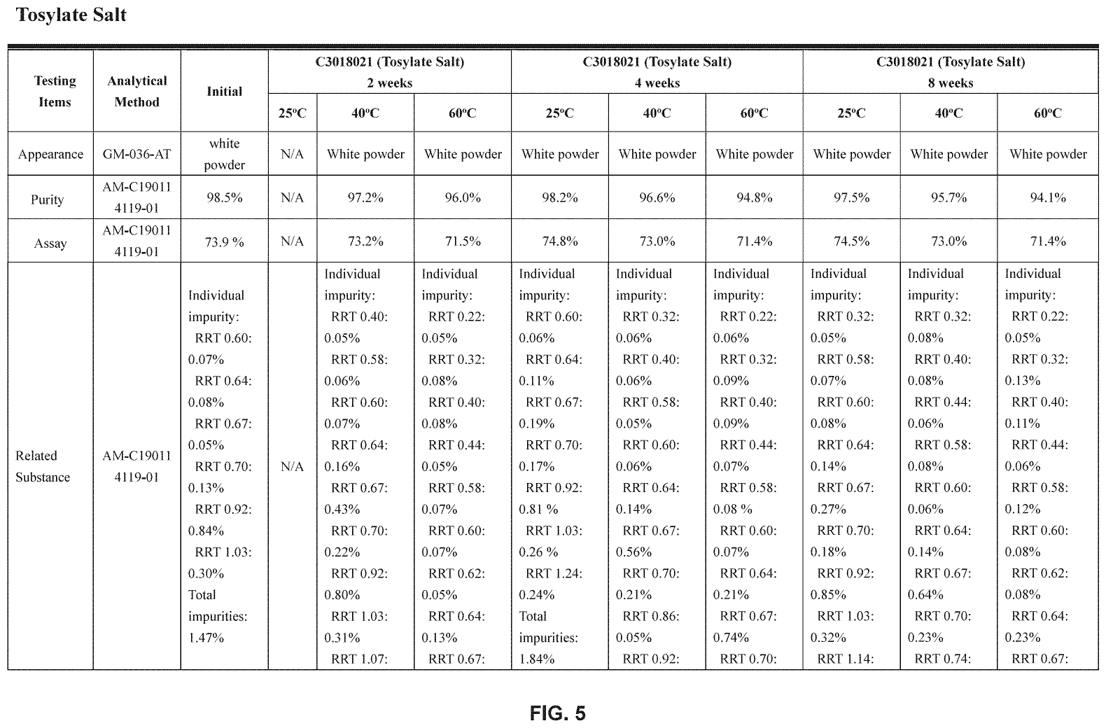

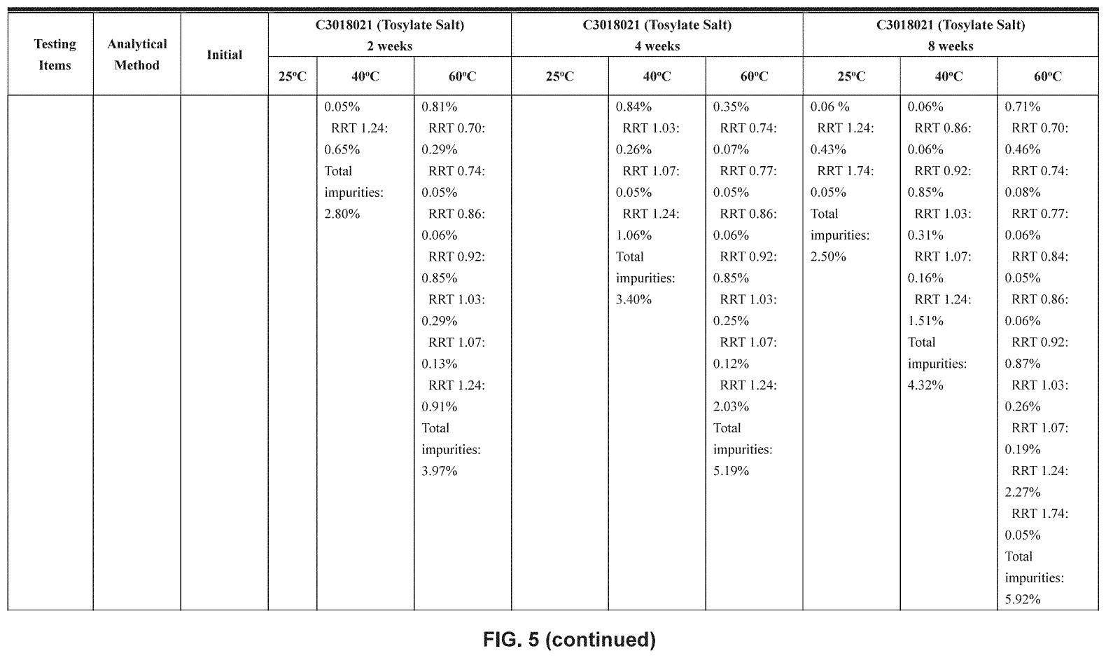

In various embodiments, the pharmaceutical composition comprises a hydrochloride salt of TBR-760, such as the trihydrochloride salt of TBR-760, and has a purity profile as shown in FIG. 2.

In certain embodiments, the pharmaceutical composition comprises a hydrochloride salt of TBR-760, such as the trihydrochloride salt of TBR-760, and less than 5% of TBR-760 is degraded after 8 weeks of storage at 60° C. In certain embodiments, less than 4.5%, 4.0%, 3.5%, 3.0%, 2.5%, 2.0%, 1.5% or 1.0% of TBR-760 is degraded after 8 weeks of storage at 60° C. In a particular embodiment, less than 1.0% of TBR-760 is degraded after 4 weeks of storage at 60° C. In certain embodiments, less than 3.0% of TBR-760 is degraded after 8 weeks.

In certain embodiments, the pharmaceutical composition comprises a hydrochloride salt of TBR-760, such as the trihydrochloride salt of TBR-760, and less than 5% of TBR-760 is degraded after 4 weeks of storage at 60° C. In certain embodiments, less than 4.5%, 4.0%, 3.5%, 3.0%, 2.5%, 2.0%, 1.5%, 1.0%, 0.9%, 0.8%, 0.7%, 0.6% or 0.5% of TBR-760 is degraded after 4 weeks of storage at 60° C. In particular embodiments, less than 1.0% of TBR-760 is degraded after 4 weeks of storage at 60° C.

In certain embodiments, the pharmaceutical composition comprises a hydrochloride salt of TBR-760, such as the trihydrochloride salt of TBR-760, and less than 5% of TBR-760 is degraded after 4 weeks of storage at 40° C. In certain embodiments, less than 4.5%, 4.0%, 3.5%, 3.0%, 2.5%, 2.0%, 1.5%, 1.0% or 0.5% of TBR-760 is degraded after 4 weeks of storage at 40° C. In particular embodiments, less than 0.8% of TBR-760 is degraded after 4 weeks of storage at 40° C. In a particular embodiment, less than 0.5% of TBR-760 is degraded after 4 weeks of storage at 40° C.

In certain embodiments, the pharmaceutical composition comprises a hydrochloride salt of TBR-760, such as the trihydrochloride salt of TBR-760, and less than 5% of TBR-760 is degraded after 4 weeks of storage at 25° C. In certain embodiments, less than 4.5%, 4.0%, 3.5%, 3.0%, 2.5%, 2.0%, 1.5%, 1.0% or 0.5% of TBR-760 is degraded after 4 weeks of storage at 25° C. In certain embodiments, less than 0.45%, 0.40%, 0.35%, 0.30%, 0.25%, 0.20%, 0.15%, 0.10%, or 0.05% of TBR-760 is degraded after 4 weeks of storage at 25° C. In certain embodiments, less than 0.09%, 0.08%, 0.07%, 0.06%, 0.05% or 0.04% of TBR-760 is degraded after 4 weeks of storage at 25° C. In a particular embodiment, less than 0.5% of TBR-760 is degraded after 4 weeks of storage at 25° C.

In some embodiments, degradation products of the pharmaceutical composition do not include N-acetyl-lysine.

In some embodiments, the pharmaceutical composition disclosed herein is a lyophilate. In some embodiments, the pharmaceutical composition is an aqueous solution.

### 5.4. Dosage Forms

In another aspect, dosage forms are provided. The dosage forms comprise the pharmaceutical compositions disclosed herein, packaged in a container. In certain embodiments, the dosage form is a unit dosage form sufficient for a single administration.

In some embodiments, the pharmaceutical compositions disclosed herein are provided as a lyophilized product in a container, for reconstitution before administration. In some embodiments, the pharmaceutical compositions are provided as an aqueous solution in a container. In certain embodiments, the compositions are packaged in a glass container, such as a glass vial, preferably an amber glass vial (for example, Type II). In certain embodiments, the compositions are packaged in a plastic container made from polyethylene, polypropylene or a combination thereof. In various embodiments, the container ranges in size from about 0.5 ml to 10 ml. In certain embodiments, the container is an ampoule.

In some embodiments, the pharmaceutical compositions are packaged in pre-filled syringes. In certain embodiments, the pre-filled syringe is transparent or translucent. In particular embodiments, the syringe is transparent. In particular embodiments, the syringe is a transparent glass syringe.

In further embodiments, the composition can be provided as a lyophilized powder in a dual chamber syringe, one chamber of which contains the lyophilized composition, the other chamber of which contains a diluent.

In various embodiments, the pharmaceutical composition is packaged at a concentration that requires dilution prior to administration.

In various embodiments, the pharmaceutical composition is packaged at a concentration suitable for administration without dilution (ready to use).

In some embodiments, the ready to use pharmaceutical composition is packaged in a container that is amenable to delivery of the ready to use pharmaceutical composition. in various embodiments, the container is a 1 ml, 2 ml, 5 ml, 10 ml glass vial or plastic vial. In some embodiments, the ready to use pharmaceutical composition is provided in a pre-filled syringe. Such pre-filled syringes are able to deliver drug solution volumes from about 0.1 ml to about 10 ml. For example, about 0.1 ml, 0.2 ml, 0.25 ml, 0.3 ml, 0.4 ml, 0.5 ml, 0.75 ml, 1.0 ml, 1.25 ml, 1.5 ml, 2.0 ml, 3.0 ml, 5.0 ml, 10 ml, of drug solution volume can be delivered to patients.

The containers of said vials or pre-filled syringes are typically made of non-reacting glass, or non-reacting polymeric material such as polypropylene or polyethylene or a mixture thereof. Several non-reacting containers are known in the art. The non-reacting containers described herein include not only the vial or pre-filled syringe that makes up the bulk of the container structure, but also any other part of the container that comes in contact with the drug solution, such as stoppers, plungers, etc. These also are made of non-reacting glass or polymeric materials.

In some embodiments, the compositions disclosed herein can be dispensed by pre-filled syringe fully assembled into an auto-injector device.

In some embodiments, the compositions disclosed herein can be dispensed in disposable, single-use auto-injector in a pre-filled syringe (PFS) fully assembled for ready use.

In a particular embodiment, the container is a pre-filled syringe. The pre-filled syringe is made up of a material having at least one non-glass component. The barrel of the pre-filled syringe is preferably made up of appropriate plastic or polymeric material. In one aspect, the syringe comprises a barrel made up of cyclic olefin polymer, cyclic olefin copolymer, polypropylene, polycarbonate and the like. The syringe may further comprise an elastomeric tip cap, made up of material such as chloro-butyl formulation. The syringe may comprise a plunger stopper made up of rubber material such as bromo-butyl rubber. The syringe may be further packaged in a secondary packaging to protect from light. The secondary packaging comprises a suitable pouch, such as an aluminum pouch and a carton packaging. The pouch may further contain an oxygen scavenger.

In some embodiments, the compositions disclosed herein can be dispensed in suitable devices that are suitable for containment and administration. Further, the devices are packed in suitable secondary package a material that envelops the devices. The secondary packaging provides additional barriers to elements that can degrade the composition such as light and oxygen. Some devices may also be designed to permeable to oxygen and other gases. For example, syringes, cartridges and the like can have permeable parts to allow sterilization process with, for example, ethylene oxide.

In some embodiments, the compositions disclosed herein comprise a secondary packaging in addition to the dispensed devices. Secondary packaging includes any container that receives the device (e.g., a box, bag, blister, canister, bottle and the like) and is sealed to prevent ingress of oxygen. The secondary packaging is made from material that has very low permeability to oxygen molecules (e.g., ethylene vinyl alcohol, aluminum, glass, polyamide and the like). In certain instances, the secondary packaging further comprises an oxygen absorber inside. The oxygen absorber functions to absorb any oxygen present in the secondary packaging. Suitable materials for oxygen absorbers include iron, low molecular weight organic compounds such as ascorbic acid and sodium ascorbate and polymeric materials incorporating a resin and a catalyst. Oxygen absorbers are contemplated to be in any size or shape including sachet, pouch, canister, lining, sticker, etc. as well as part of the secondary packaging or primary packaging container itself.

In certain embodiments the syringe can be a glass syringe, such as a prefilled glass luer syringe with 4023/50 FluroTec coated plunger.

### 5.5. Kits

In a further aspect, kits are provided. The kits comprise (a) the pharmaceutical composition as described herein, and (b) a diluent.

In some embodiments, the kit further comprises (c) an injection syringe; (d) a vial comprising the pharmaceutical composition as a lyophilate; (e) a vial comprising the diluent; and (f) a transfer syringe. In particular embodiments, the diluent comprises water, trehalose, and pH adjusters.

### 5.6. Synthesis of Somatostatin-Dopamine Chimeric Analogs

5.6.1. Somatostatin Agonists

Somatostatin agonists can be synthesized according to methods known to those of skill in the art and as described in U.S. Pat. No. 7,517,853 and European Patent Application EP0395417, both of which are incorporated herein in their entirety. Briefly, peptides are synthesized on Rink amide MBHA resin (4-(2′ 4′-dimethoxyphenyl-Fmoc-aminomethyl)-phenoxyacetamido-nor-leucyl-MIBHA resin) using a standard solid phase protocol of Fmoc chemistry. The peptide-resin with free amino functional at the N-terminus is then treated with the corresponding compound containing dopamine moiety. The final product is cleaved off from resin with TFA water/triisopropylsilane (TIS) mixture.

The synthesis of somatostatin agonists with a substituted N-terminus can be achieved, for example, by following the protocol set forth in PCT Publication Nos. WO 88/02756, WO 94/04752, and/or European Patent Application No. EP 0329295, each of which is hereby incorporated by reference in its entirety.

Peptides can be cyclized by using iodine solution in MeOH/water and purified on C18 reverse-phase preparative high performance liquid chromatography (HPLC) using acetonitrile-0.1% TFA/water-0.1% TFA buffers. Homogeneity is assessed by analytical HPLC and mass spectrometry.

5.6.2. Synthesis of Dopamine Agonists

Methods for synthesizing many dopamine agonists are well documented and are within the ability of persons of ordinary skill in the art. Further procedures for synthesizing dopamine receptors are provided in the reaction schemes and examples shown in U.S. Pat. No. 7,517,853, hereby incorporated by reference in its entirety.

5.6.3. Synthesis of Somatostatin-Dopamine Chimeric Analogs

Somatostatin-dopamine chimeras may be synthesized according to the reaction schemes and examples provided in U.S. Pat. Nos. 7,517,853 and 8,822,442, both of which are hereby incorporated by reference in their entirety.

### 5.7. Methods of Treatment

Also provided herein are methods for treating neuroendocrine tumors and endocrine diseases, including, but not limited to, non-functioning pituitary adenomas (NFPA). The methods comprise administering a therapeutically or prophylactically effective amount of a storage-stable salt of a somatostatin-dopamine chimeric analog to a subject who has, or is at risk of developing, an endocrine tumor or endocrine disease. In typical embodiments, the methods comprise administering a therapeutically or prophylactically effective amount of a storage-stable pharmaceutical composition comprising a storage-stable salt of a somatostatin-dopamine chimeric analog.

In certain embodiments, the somatostatin-dopamine chimeric analog is TBR-760. In particular embodiments, the storage-stable salt is the hydrochloride salt of TBR-760. In particular embodiments, the storage-stable salt is the trihydrochloride salt of TBR-760.

In typical embodiments the subject has previously been diagnosed with a non-functioning pituitary adenoma (NFPA). In some embodiments, the patient has a non-functioning pituitary adenoma (NFPA).

In certain embodiments, the subject is a mammal. In certain embodiments the subject is a human. In some embodiments the subject is an adult. In certain other embodiments the subject is a child.

In various embodiment, the storage-stable somatostatin-dopamine chimeric analog or pharmaceutical composition comprising the same is administered intravenously. In some embodiments, the storage-stable somatostatin-dopamine chimeric analog or pharmaceutical composition comprising the same is administered by intrathecal administration, intra-cisterna magna administration, intraventricular administration, subcutaneous administration, intramuscular administration, or intraperitoneal administration. In certain embodiments, the storage-stable somatostatin-dopamine chimeric analog or pharmaceutical composition comprising the same is administered peritumorally or intratumorally. In certain embodiments, the storage-stable somatostatin-dopamine chimeric analog or pharmaceutical composition comprising the same is administered intranasally. In certain embodiments, the storage-stable somatostatin-dopamine chimeric analog or pharmaceutical composition comprising the same is administered orally.

In various embodiments, the pharmaceutical composition comprising TBR-760 is administered in an amount, on a schedule, and for a duration sufficient to reduce tumor growth in the subject. In some embodiments, the pharmaceutical composition is administered in an amount, on a schedule, and for a duration sufficient to decrease tumor volume and/or tumor diameters by 5%, 10%, 15%, 20%, 25%, 30%, 35% or more as compared to tumor size just prior to initiation of treatment. In certain embodiments, the pharmaceutical composition is administered in an amount, on a dosage schedule, and for a duration sufficient to decrease tumor volume and/or tumor diameter by 40%, 45%, 50%, 55%, 60% or more. In particular embodiments, the pharmaceutical composition is administered in an amount, on a schedule, and for a time sufficient to decrease tumor volume and/or tumor diameter by 65%, 70%, 75%, 80%, 85%, 90%, 95% or more.

In various embodiments, the pharmaceutical composition comprising TBR-760 is administered in an amount, on a schedule, and for a duration sufficient to inhibit cell proliferation. In some embodiments, the pharmaceutical composition is administered in an amount, on a schedule, and for a duration sufficient to inhibit tumor cell growth by 5%, 10%, 15%, 20%, 25%, 30%, 35% as compared to tumor cells treated with a control. In certain embodiments, the pharmaceutical composition is administered in an amount, on a dosage schedule, and for a duration sufficient to inhibit tumor cell growth by 40%, 45%, 50%, 55%, 60% or more. In particular embodiments, the pharmaceutical composition is administered in an amount, on a schedule, and for a time sufficient to inhibit tumor cell growth by 65%, 70%, 75%, 80%, 85%, 90%, 95% or more.

In some embodiments, the methods comprise administering the pharmaceutical composition comprising TBR-760 in combination with a second treatment, either simultaneously or sequentially, dependent upon the condition to be treated. The second treatment may include, but is not limited to, other dopamine and/or somatostatin receptor agonists, hormone therapy, radiation treatment, and surgery.

In another aspect, the disclosure provides compounds or compositions for use in therapy or as a medicament. The disclosure further provides compounds or compositions for use in the treatment of an endocrine disease or endocrine tumor. The disclosure also provides the use of compounds or compositions in the manufacture of a medicament for the treatment of an endocrine disease or endocrine tumor.

Administration of the pharmaceutical composition of the present disclosure is preferably in a “therapeutically effective amount” or “prophylactically effective amount, this being sufficient to show benefit to the individual. The actual amount administered, and rate and time-course of administration, will depend on the nature and severity of the endocrine disease or tumor being treated. Prescription of treatment, e.g., decisions on dosage etc., is within the responsibility of general practitioners and other medical doctors, and typically takes account of the disease or disorder to be treated, the condition of the individual patient, the site of delivery, the method of administration and other factors known to practitioners.

### 5.8. Examples

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature.

### Example 1: Preparation of TBR-760 and Lanreotide Salt Forms

TBR-760 (Dop2-D-Lys(Dop2)-cyclo[Cys-Tyr-D-Trp-Lys-Abu-Cys]-Thr-NH₂) was made as described in U.S. Pat. Nos. 7,517,853 and 8,822,442, each of which is hereby incorporated by reference in its entirety.

In order to exchange the acetate salt with hydrochloric acid (HCl), a solution of 2 grams (1.07 mmoles) of TBR-760 acetate in 100 mL of 20% acetonitrile in water was prepared in a 500 mL lyophilization flask. 3 equivalents of HCl (267 μL, from 12 N HCl) was added to the chimeric peptide solution, stirred for approximately 10 minutes, and lyophilized. The process was separately repeated with addition of neat acid and/or concentrated acid solution to generate the sulfate, mesylate, and tosylate salt forms.

TBR-760 bromide, lanreotide bromide, and lanreotide chloride were prepared by two-step ion-exchange. Briefly, with reference to preparation of TBR-760 bromide, Amberlyst A26 hydroxide form resin was treated with excessive HBr solution (2.2 M) to generate Br form resin. Wet resin was loaded on a 15 mm diameter column to 2.5 cm high and flushed with water to remove excess HBr. Approximately 40 mg of TBR-760 acetate salt was dissolved in approximately 6 mL of water and loaded on the column. Eluent was collected a 1 drop per second. Initial eluent of the same volume was collected and passed through the column two more times. Eluent was subsequently collected in small fractions of approximately 2 mL each. 10 μL of each fraction was diluted 10 times with water and tested for UV absorbance at λₘₐₓ=285 nm. Fractions were combined, aliquoted in 20 mL glass vials with approximately 7 mL fill and freeze-dried using a manifold freeze-drier.

Purity of the TBR-760 and lanreotide salts was assessed by ultra-high performance liquid chromatography (UPLC). Each compound was confirmed to be TBR-760 or lanreotide by mass spectrometry electron spray ionization (MS-ESI), which identified the predominant peak of each salt evaluated as correlating with the expected molecular weight of TBR-760 (1694.2) or lanreotide.

### Example 2: Time and Temperature Stability Studies of TBR-760 Salts

Lyophilized TBR-760 salts prepared as described above were assessed for stability by visual appearance and HPLC following initial production of the salt and following incubation of the salt at 20° C., 40° C., and 60° C. for 2, 4, and 8 weeks in sealed, glass vials. Changes in visual appearance of the salt were noted by evaluating the color of the substance following each incubation condition. Purity of the salt following each incubation condition was evaluated by HPLC. The number and quantity of degradation products were determined according to the number of absorbance peaks that resulted at different retention times and the size of the peaks.

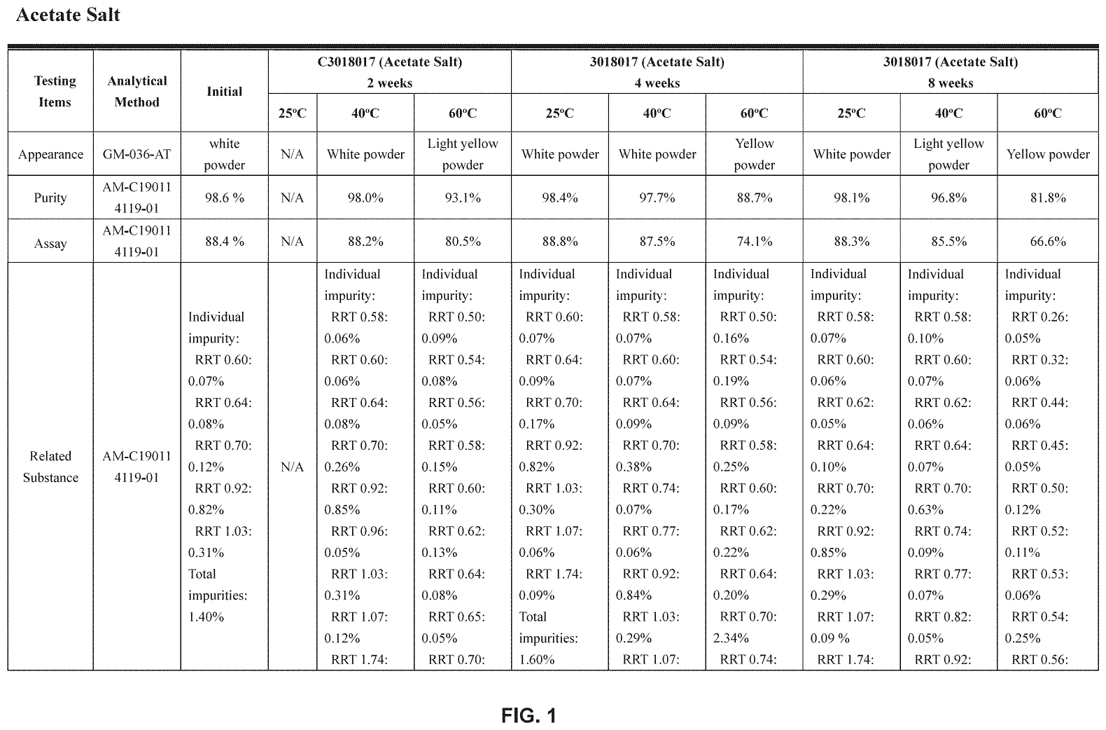

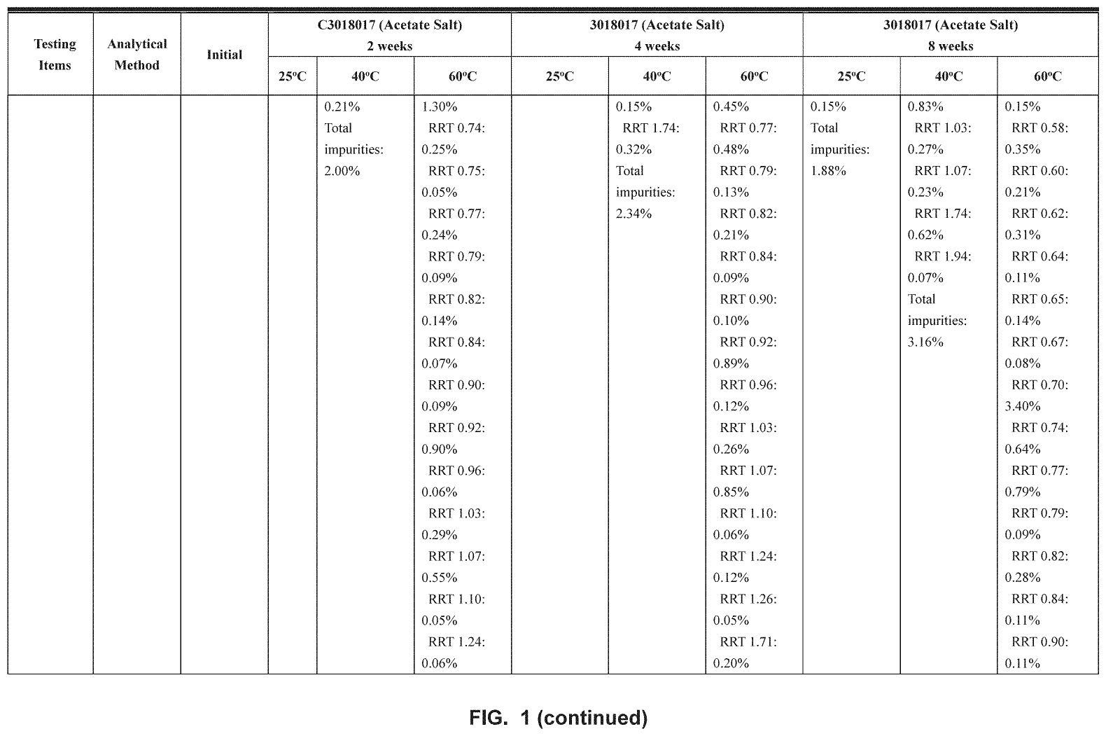

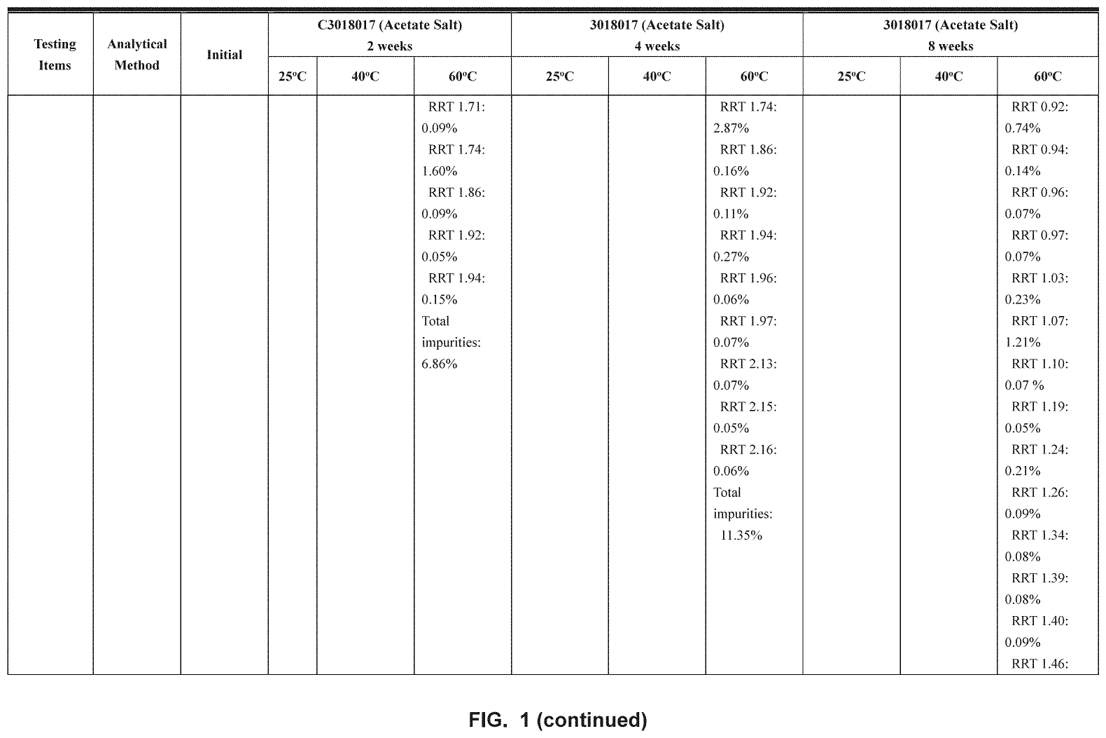

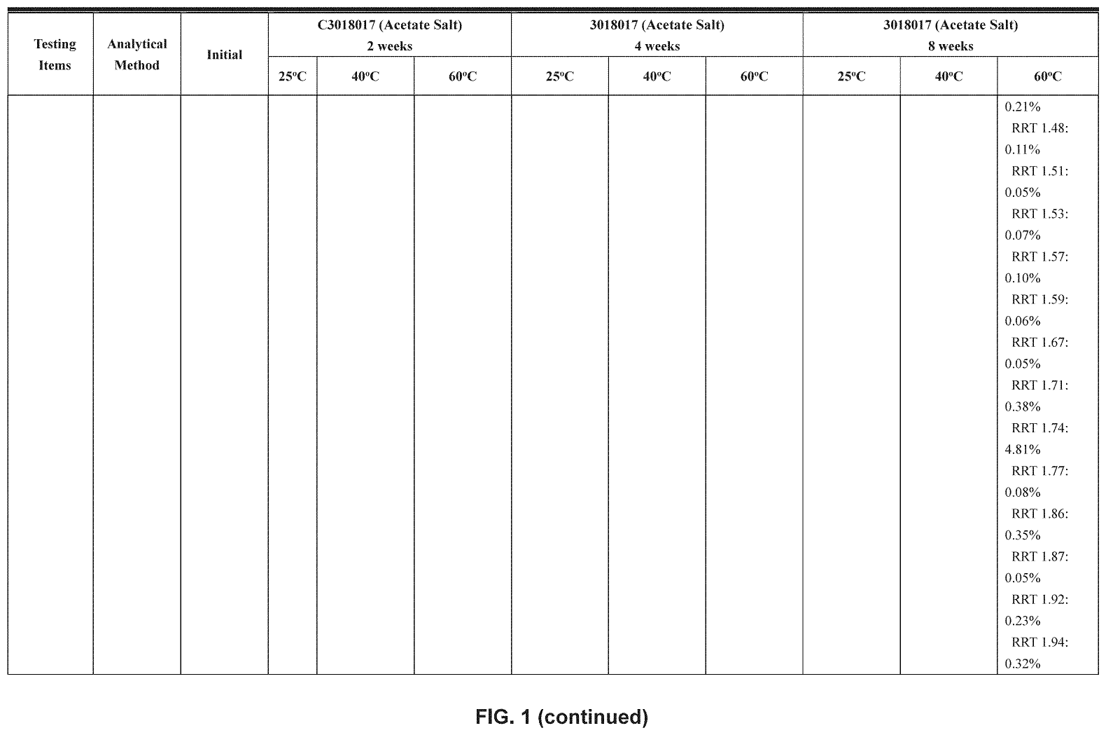

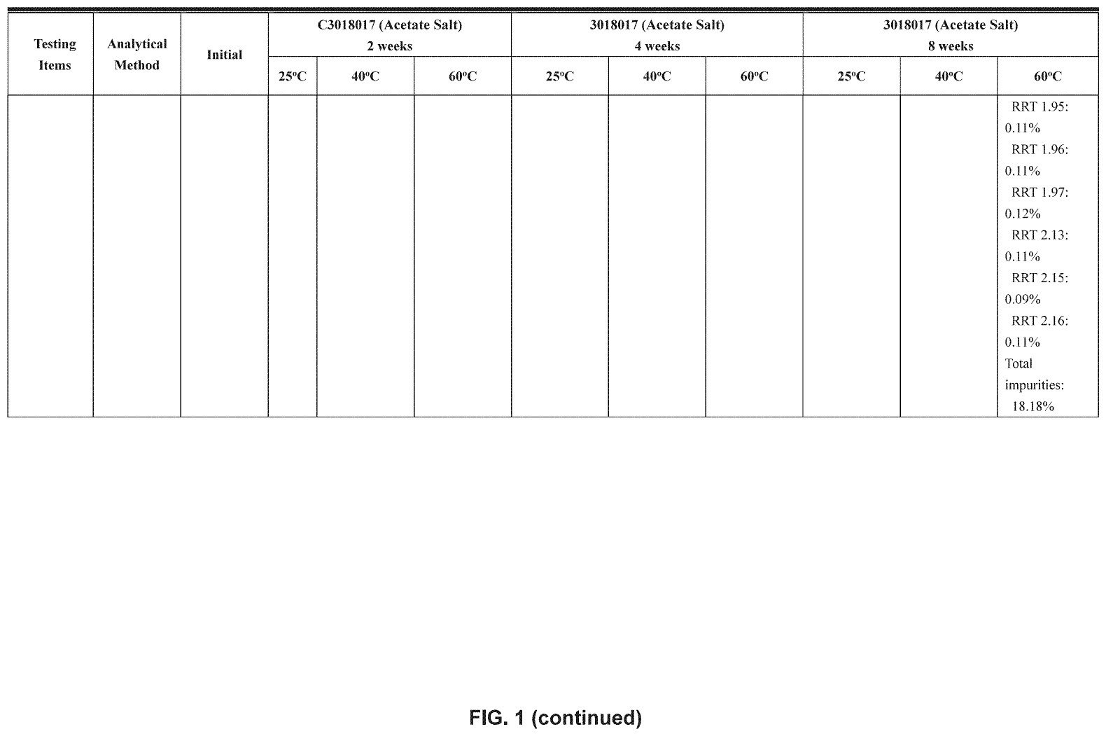

Results (FIGS. 1-2) show that at 60° C. for 8 weeks, more than 16% of the TBR-760 acetate salt form is degraded, compared to less than 1% degradation of the TBR-760 chloride salt form under the same conditions. Similarly, at 60° C. for 4 weeks, 9.9% of the TBR-760 acetate salt form is degraded, compared to 0.5% degradation of the TBR-760 chloride salt form under the same conditions. The change in the visual appearance of the acetate salt from a white powder to a yellow powder over 4 weeks at 60° C. further demonstrates degradation of the acetate salt (FIG. 1). In comparison, there is no change in the visual appearance of the chloride salt when incubated for a longer period of 8 weeks at 60° C. (FIG. 2).

### Example 3: Pharmacokinetic Assessment of TBR-760 Salts in Rats

In order to assess the pharmacokinetic properties of the acetate, chloride, sulfate, mesylate, and tosylate salt forms of TBR-760 after subcutaneous administration, each salt form was separately administered by subcutaneous (SC) injection to male Sprague Dawley rats that had jugular vein cannulas. On the day of dosing, each salt was reconstituted in 8% trehalose in sterile water and administered to animals as provided in Table 1.

TABLE 1

| | | | | Conc. | | | |
|---|---|---|---|---|---|---|---|
| | | | | Dosing | | Vol. | |
| | | | Dose | solution | Correction | injection | Route of |
| Group | Number | Substance | (mg/kg) | (mg/mL) | Factor | (mL/kg) | administration |
| 1 | 3 | TBR-760 | 5.0 | 5.0 | 1.12 | 1.0 | SC injection |
| | | acetate | | | | | |
| 2 | 3 | TBR-760 | 5.0 | 5.0 | 1.17 | 1.0 | SC injection |
| | | chloride | | | | | |
| 3 | 3 | TBR-760 | 5.0 | 5.0 | 1.10 | 1.0 | SC injection |
| | | sulfate | | | | | |
| 4 | 3 | TBR-760 | 5.0 | 5.0 | 1.08 | 1.0 | SC injection |
| | | mesylate | | | | | |
| 5 | 3 | TBR-760 | 5.0 | 5.0 | 1.35 | 1.0 | SC injection |
| | | tosylate | | | | | |

Animals received food and water ad libitum throughout the study. Blood samples were collected from awake animals via the jugular vein cannula at pre-dose and at 2 hours, 4 hours, 8 hours, 12 hours, 24 hours, 30 hours, 48 hours, and 72 hours after dosing into K₂EDTA vials (˜50 μL blood per sample) and kept on ice until processed to isolate the plasma (centrifugation at 4° C., 2500×g for 10 min). The plasma samples were aliquoted into separate 1.5 mL Eppendorf vials and stored at −80° C. until analysis.

Plasma samples were mixed with a solution of acetonitrile/ultrapure water (90:10) containing 1 μM propranolol (Internal Standard). After 5 minutes incubation and mixing at room temperature, the samples were centrifuged for 5 minutes (17949×g, 4° C.) and the supernatants were diluted 10-fold in ultrapure water with 0.1% formic acid.

Diluted plasma supernatants (20 μL) were injected into a Prominence HPLC system (Shimadzu) by an automated sample injector (SIL-20ADHT, Shimadzu). Concentrations of TBR-760 in plasma samples were quantified by liquid chromatography with tandem mass spectrometry (LC-MS/MS) detection in multiple-reaction-monitoring mode (MRM).

Analytes were separated using a linear gradient of mobile phase B at a flow rate of 0.200 mL/min on a reversed phase Kintex Biphenyl column (100*2.1 mm, 2.6 μm particle size; Phenomenex) held at room temperature of 45° C. Mobile phase A consisted of ultrapure water with 10 mM ammonium formate and 0.1% formic acid. Mobile phase B was acetonitrile with 0.1% formic acid.

Acquisitions were achieved in positive ionization mode using an API 4000 (Applied Biosystems) equipped with a Turbo Ion Spray interface (positive ionization mode). The ion spray voltage was set at 5.0 kV and the probe temperature was 500° C. The collision gas (nitrogen) pressure was kept at 2 psi. The following MRM transitions were used for quantification of propranolol: m/z 260.0/183.0 and TBR-760 m/z 565.6/129.0. Data were calibrated and quantified using the Analyst data system (Applied Biosystems, version 1.4.2).

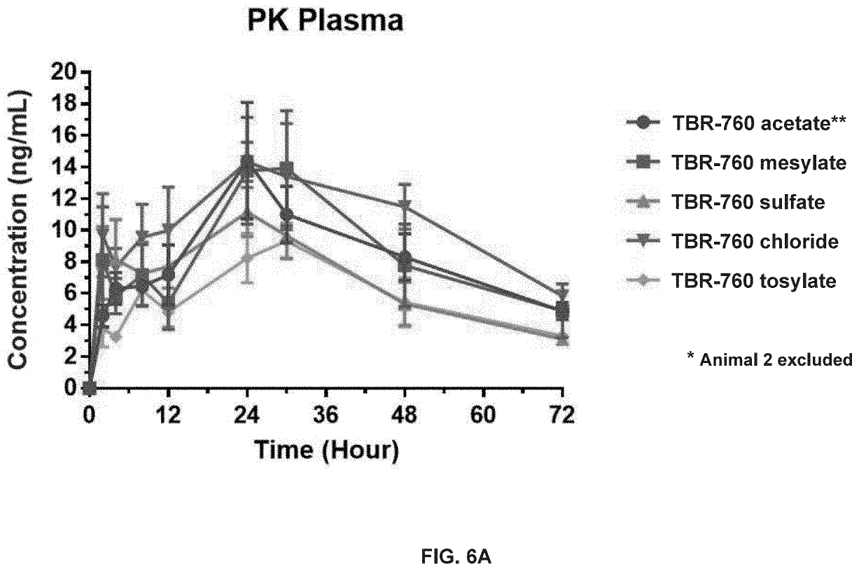

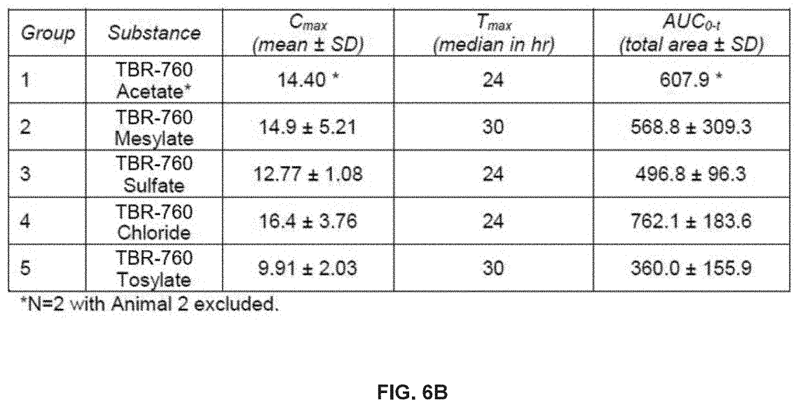

The maximum serum concentration (Cₘₐₓ), time elapsed to maximum concentration (Tₘₐₓ), and AUC₀₋ₜ of TBR-760 acetate, TBR-760 mesylate, TBR-760 sulfate, TBR-760 chloride, and TBR-760 tosylate is shown in FIG. 6B. Plasma concentration of each salt form over time is shown in FIG. 6A. The results show that the salt form of TBR-760 does not appear to affect the pharmacokinetics of the compound when administered s.c. to rats.

### Example 4: Evaluating Tolerability of Subcutaneous Injection of TBR-760 Salt Forms in Göttingen Minipigs

In order to evaluate the tolerability of various salt forms of TBR-760, each salt form was administered by subcutaneous injection to female Göttingen minipigs at a different marked injection site. Each salt was separately reconstituted in 8% trehalose. Subcutaneous doses of each salt form were administered with a syringe and appropriately sized needle in the tented skin on the abdomen of the animal at a volume of 0.1 mL/kg. The dose volume was adjusted for the weight of the animal on the day of the dosing.

Doses were administered once a day for 7 days. Three doses were administered in the morning (Injection Sites 1, 2, and 4) while the remaining two doses (Injection Sites 3 and 5) were administered in the afternoon. The TBR-760 salt forms were administered to the animals at each injection site as provided in Table 2.

TABLE 2

| Injection Site | Compound Administered |
|---|---|
| 1 | TBR-760 acetate |
| 2 | TBR-760 mesylate |
| 3 | TBR-760 sulfate |
| 4 | TBR-760 chloride |
| 5 | TBR-760 tosylate |

Each injection site was evaluated daily for erythema and edema according to a modified Draize Scoring system as provided in Table 3. Daily evaluations were made predose and 2 hours postdose.

TABLE 3

| Erythema | Edema | Score |
|---|---|---|
| None | None | 0 |
| Slight | Very slight | 1 |
| Well defined | Slight (well defined edges) | 2 |
| Moderate or Severe | Moderate (raised greater than 1 mm) | 3 |
| Severe erythema or | Severe (raised greater than 1 mm and | 4 |
| slight eschar | extending beyond the area of exposure) | |
| formation | | |
| (injuries in depth) | | |

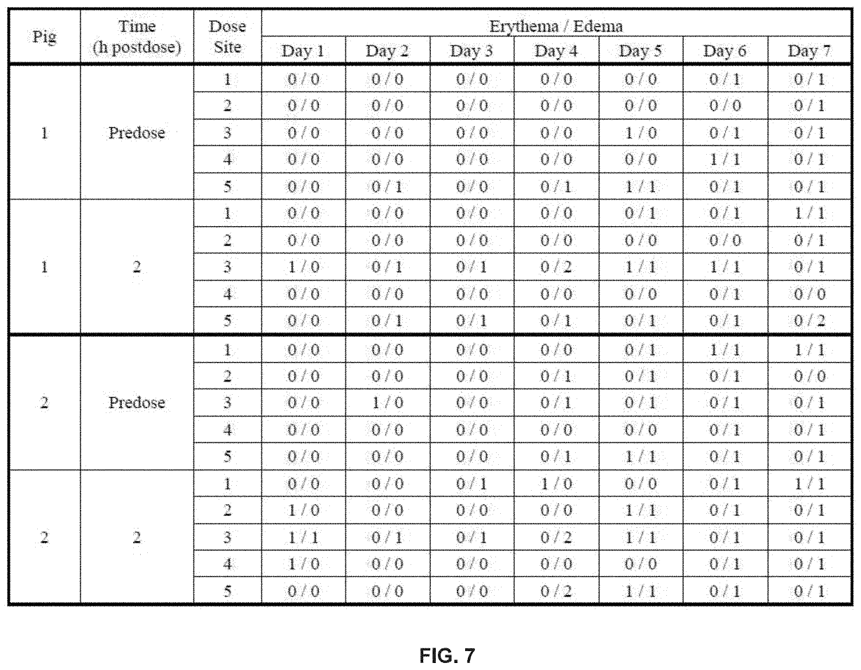

All pigs were observed daily during the 7 day study for cage side clinical signs. Pig 1 displayed slight vocalization when Injection Site 3 was palpated during Draize Scoring on Day 4. All pigs were normal all other times. Results shown in FIG. 7 show that all salt forms were well tolerated by both animals throughout the study with scores of 0 for erythema and 1 or 2 for edema.

### Example 5: Thermogravimetric Analyses and Differential Scanning Calorimetry of TBR-760 Salt Forms

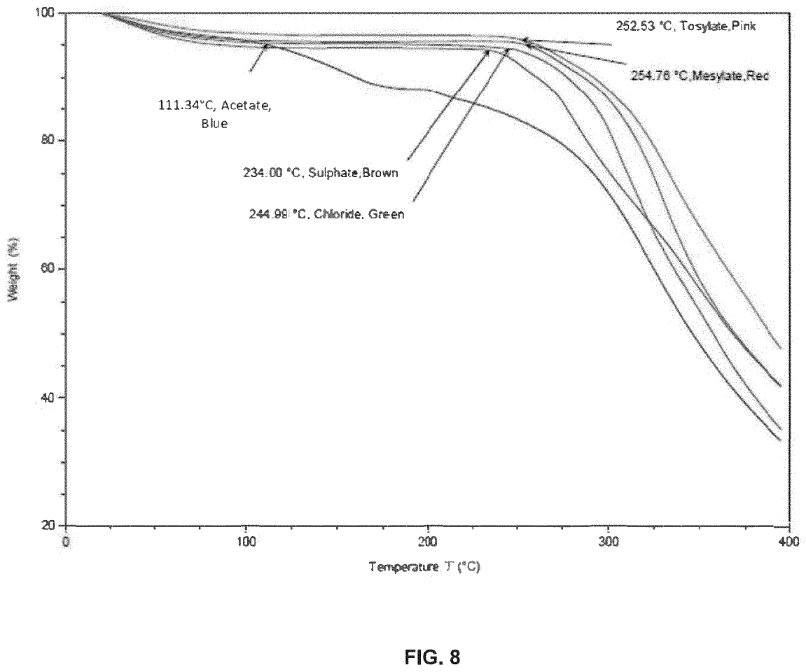

Thermogravimetric analyses (TGA) were performed on all TBR-760 salt forms to evaluate thermal stability of each salt form. Studies were performed using a TGA-Q500 thermogravimetric analyzer (TA Instruments). The weight of each compound was measured while the temperature was increased at a ramp rate of 5.0° C. per minute from 25 to 400° C. Results shown in FIG. 8 demonstrate that there was an approximately 5% loss of weight for all salt forms below 120° C. The weight loss may be indicative of loss of adsorbed moisture, entrained solvent or volatile impurities. Decomposition of the compound appears to start at about 234° C. (sulfate salt), 245° C. (chloride salt), 252° C. (tosylate salt), and 255° C. (mesylate salt). For the acetate salt, a further ˜5% loss of weight between 110° C. and 200° C. was observed, indicating loss of volatile acetate ions over that temperature range (FIG. 8).

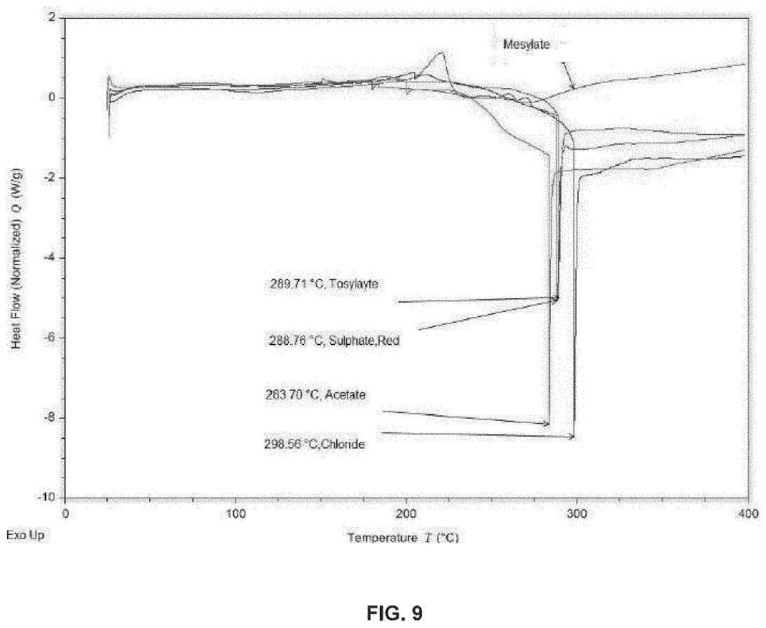

Differential scanning calorimetry (DSC) analyses were performed on all TBR-760 salt forms in order to identify any phase changes in each over a given temperature range. An amount of solid sample (0.8-1.5 mg) of each salt form was weighed on a hermetic sealed pan using an analytical balance. The pan was then sealed using a hermetic sealed lid and a manual hermetic lid press. DSC data was recorded at a ramp rate of 5.0° C. per minute from 25 to 400° C. under a nitrogen purge flowrate of 50 mL/min. Results of the DSC analyses of all five salts are shown in FIG. 9. The DSC curves of each salt form did not demonstrate any sharp glass transition or crystallization peaks. All salts except mesylate show a sharp endotherm above 280° C., consistent with the TGA analyses indicating that degradation of the salts occurs at temperatures above 234° C. (FIG. 9).

### Example 6: Solid State Stability of TBR-760 Salt Forms

Each TBR-760 salt was further evaluated for thermolytic stability over time by setting up three different exposure conditions (25° C. at 60% relative humidity (RH); 40° C. at 75% RH; or 60° C. with open and closed container systems (open dish/closed dish). Approximately 50 mg of each salt form was weighed in a nitrogen-flushed glove box for use in the studies. All samples were stored at −20° C. in a good laboratory practice (GLP) freezer both before and after exposure to the indicated conditions.

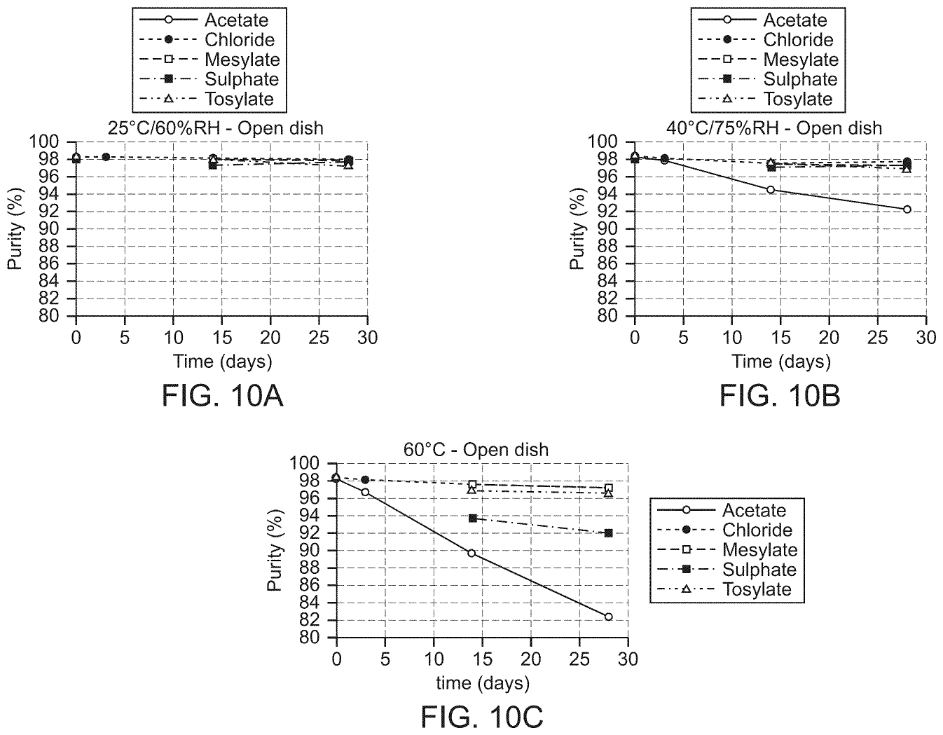

The stability of each salt form was assessed by monitoring the post-exposure drug peak purity and by evaluating the post-exposure visual appearance of the salt. The samples were removed from the exposure conditions and allowed to equilibrate to ambient conditions for 15-20 mins. An appropriate amount of each sample was weighed for analysis and dissolved in diluent (deionized water containing 0.1% TFA) to obtain a stock concentration of 1 mg/mL. The stock solution was then diluted to 0.125 mg/mL in an HPLC vial using diluent and the samples were run on a Waters Acquity UPLC system using the parameters shown in Table 4. The peak purity and the appearance of each salt form exposed to conditions in open container systems are shown in Table 5. Graphs of peak purity over time of exposure for each condition in open container systems is also shown in FIG. 10.

TABLE 4
Summary of UPLC Parameters

| Column | Name | Acquity UPLC Peptide CSH C18, 130 Å, |
|---|---|---|
| information | | 1.7 μm, 2.1 mm × 150 mm |
| | SN # | 01593920618545 |

| Mobile Phase A | Water (0.1% TFA) | | |
|---|---|---|---|
| Mobile Phase B | ACN (0.1% TFA) | | |
| Diluent | Water (0.1% TFA) | | |
| Sample Manager | 76:24 :: MP A: MP B | | |
| Wash | | | |
| Needle wash | 76:24 :: MP A: MP B | | |
| Seal Wash | 76:24 :: MP A: MP B | | |
| Column storage | 100% ACN | | |
| solution | | | |
| Flow rate | 0.2 mL/min | | |
| Column | 30° C. | | |
| temperature | | | |
| Auto sampler | 5° C. | | |
| temperature | | | |
| Detector wavelength | 220 nm | | |
| Run time | 60 min | | |
| | Time (min) | % A | % B |
| Pump gradient | Initial | 76 | 24 |
| program | 32 | 68 | 32 |
| | 40 | 40 | 60 |
| | 45 | 76 | 24 |
| | 60 | 76 | 24 |

TABLE 5
Results of Thermostability Studies

| | | Temp | | T = | T = | T = |
|---|---|---|---|---|---|---|
| Salt Form | Test | (° C.) | Control | 3 d | 2 wk | 4 wk |
| Acetate | Appearance | 25 | White | White | White | White |
| | | 40 | | White | White | Hint of light |
| | | | | | | orange |
| | | 60 | | White | Hint of | Pale Orange |
| | | | | | light | |
| | | | | | orange | |
| | Purity | 25 | 98.35 | 98.52 | 98.16 | 98.09 |
| | | 40 | | 98.4 | 94.52 | 92.37 |
| | | 60 | | 96.66 | 89.7 | 82.32 |
| Chloride | Appearance | 25 | White | White | White | White |
| | | 40 | | White | White | White |
| | | 60 | | White | White | White |
| | Purity | 25 | 98.31 | 98.44 | 98.28 | 98.04 |
| | | 40 | | 98.36 | 97.62 | 97.78 |
| | | 60 | | 98.45 | 97.58 | 97.17 |
| Mesylate | Appearance | 25 | White | White | White | White |
| (Methyl- | | 40 | | White | White | White |
| sulfonate) | | 60 | | White | White | White |
| | Purity | 25 | 98.39 | *na | 98.14 | 97.77 |
| | | 40 | | *na | 97.49 | 97.35 |
| | | 60 | | *na | 97.56 | 97.17 |
| Sulfate | Appearance | 25 | White | White | White | White |
| | | 40 | | White | White | White |
| | | 60 | | White | White | White |
| | Purity | 25 | 98.04 | *na | 97.34 | 97.75 |
| | | 40 | | *na | 97.08 | 97.3 |
| | | 60 | | *na | 93.7 | 92.01 |
| Tosylate | Appearance | 25 | White | White | White | White |
| (4-Toluene- | | 40 | | White | White | White |
| sulfonate) | | 60 | | White | White | White |
| | Purity | 25 | 98.39 | *na | 98.01 | 97.39 |
| | | 40 | | *na | 97.57 | 96.93 |
| | | 60 | | *na | 96.86 | 96.52 |

*na = not analyzed

The data demonstrate that all salt forms were mostly stable for 4 weeks when exposed to conditions of 25° C. at 60% RH. However, when the salts were exposed to 40° C. at 75% RH and to 60° C., both the chloride and mesylate salts were significantly more stable over a period of 2 and 4 weeks than the other salt forms. The acetate salt was the least stable of the salts tested, followed by the sulfate salt. The results are summarized graphically in FIG. 10.

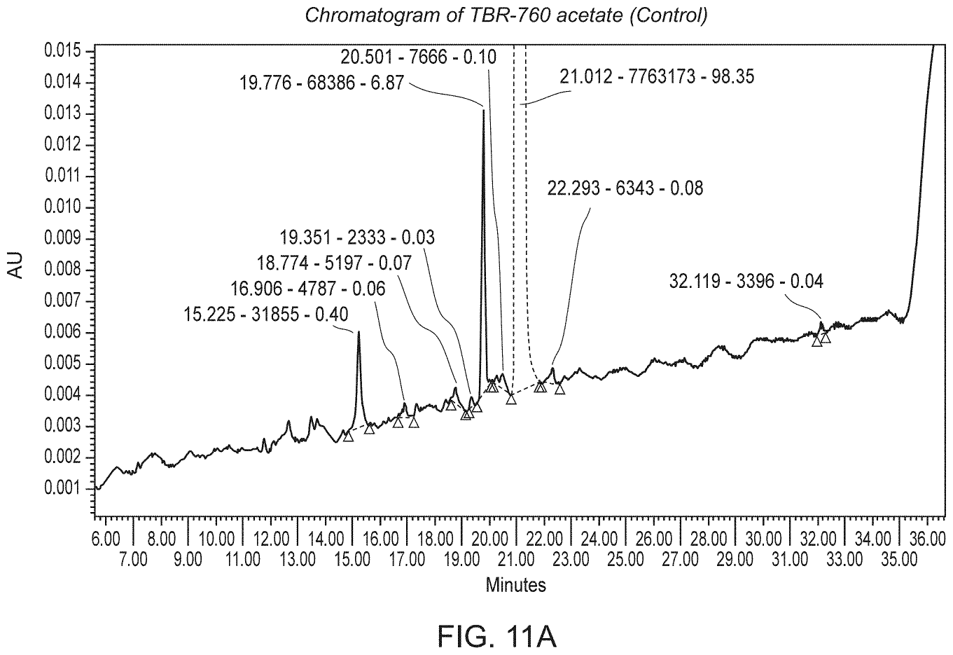

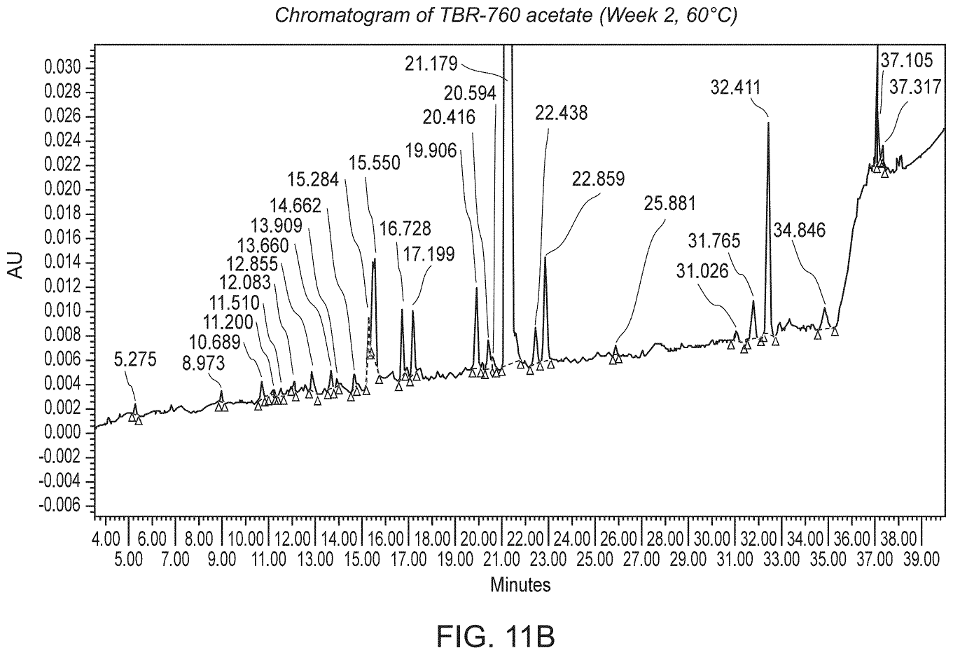

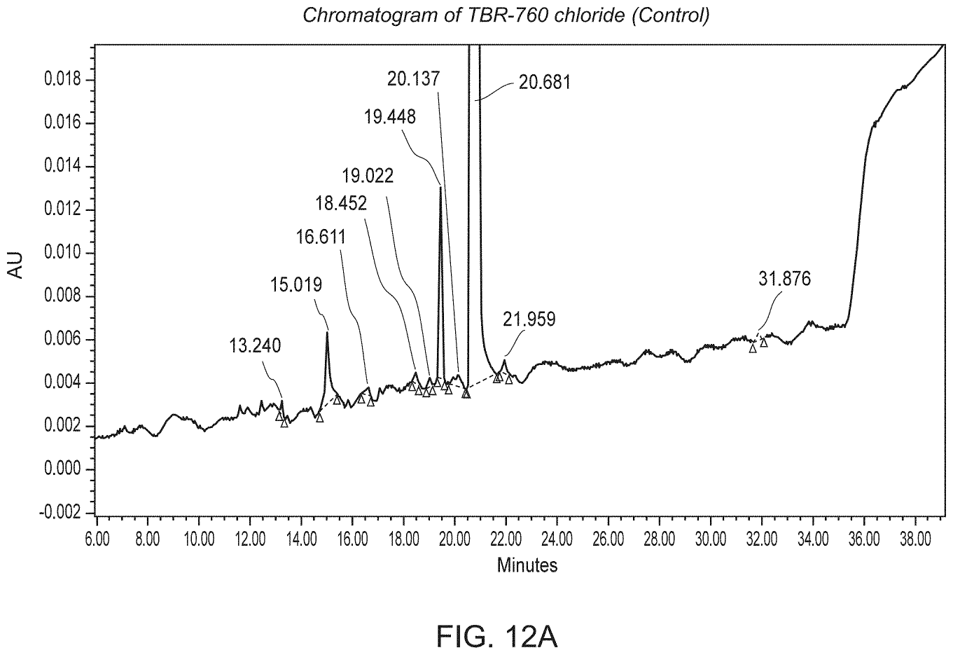

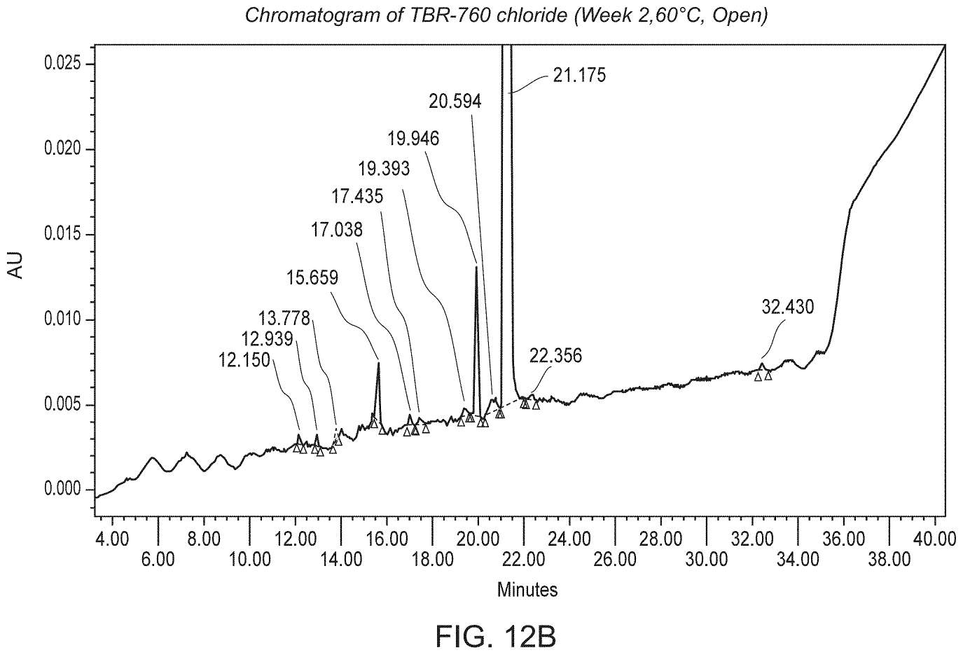

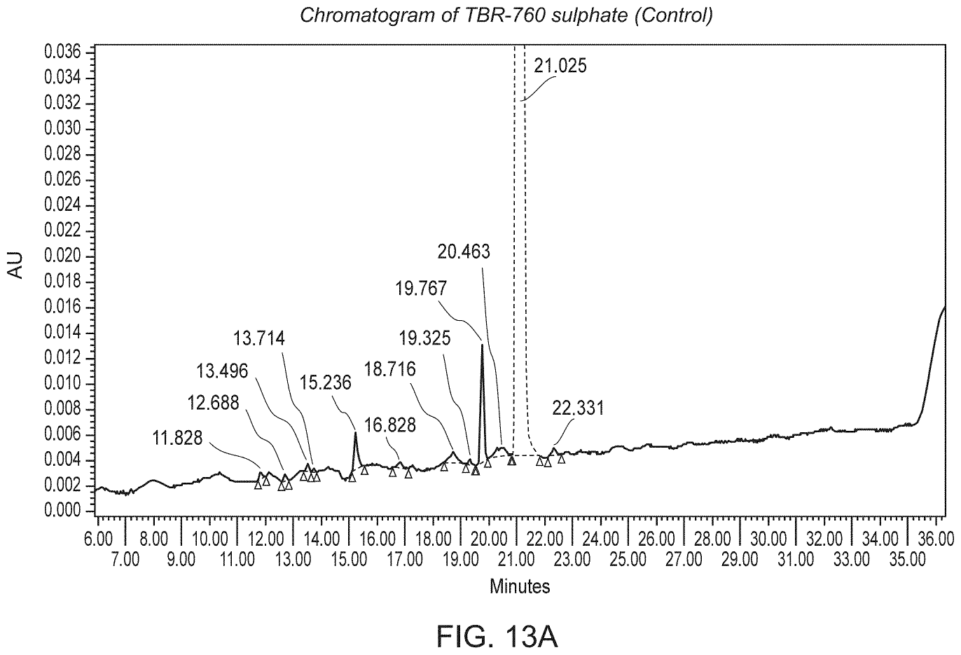

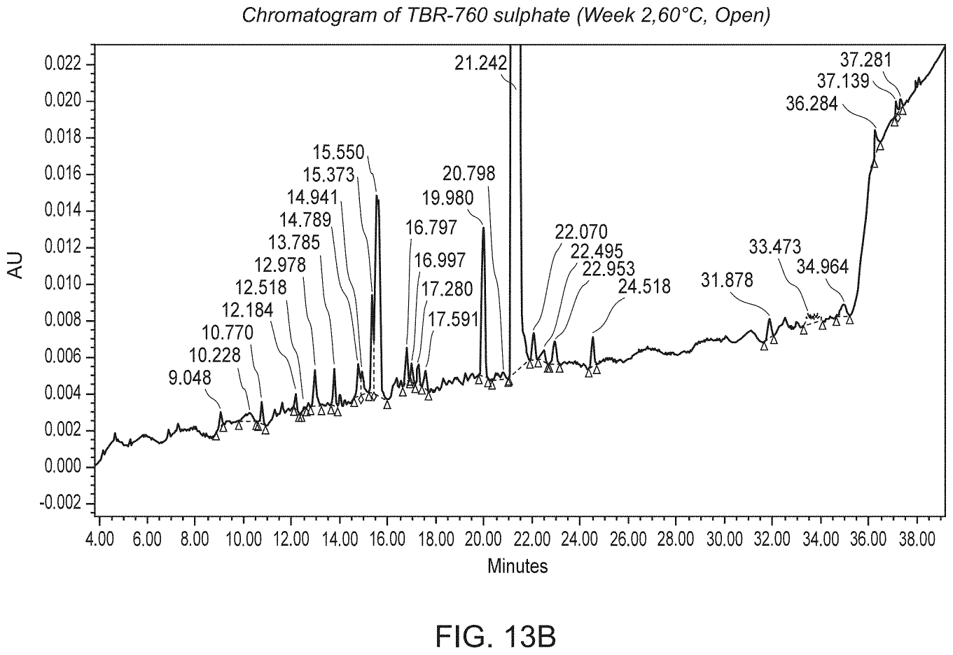

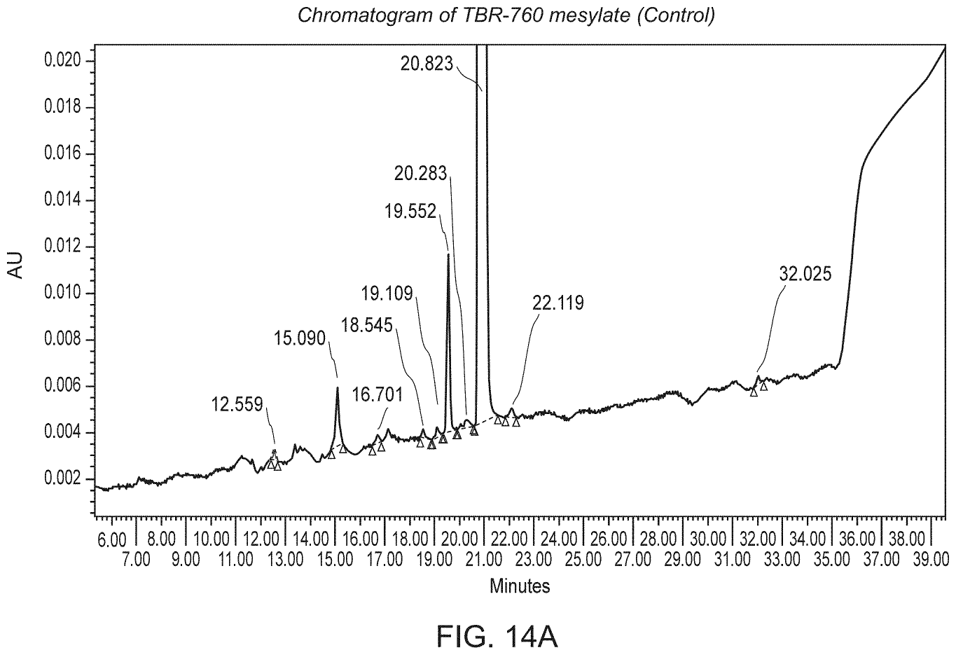

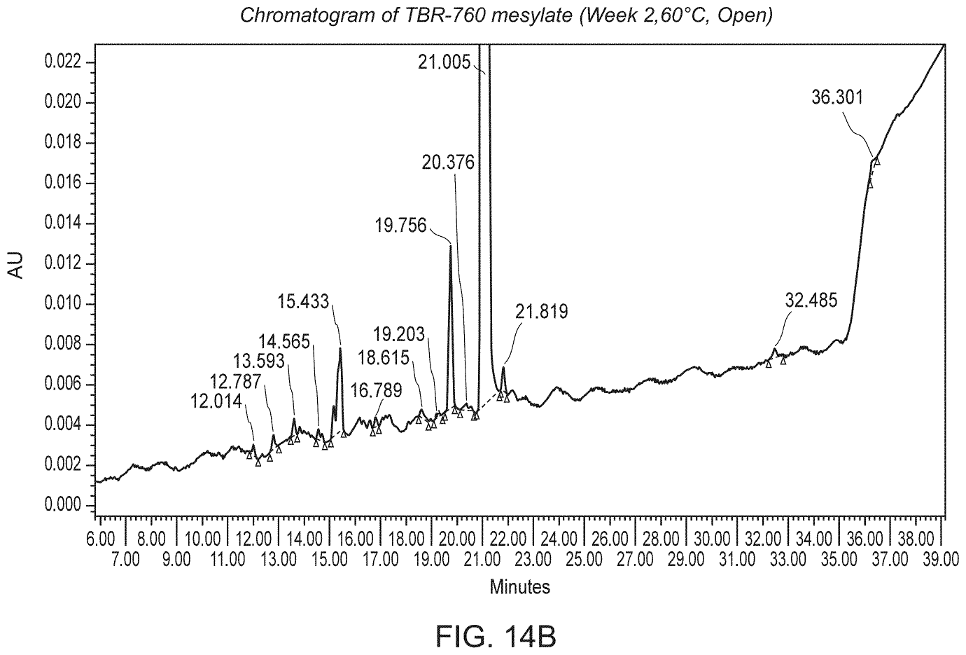

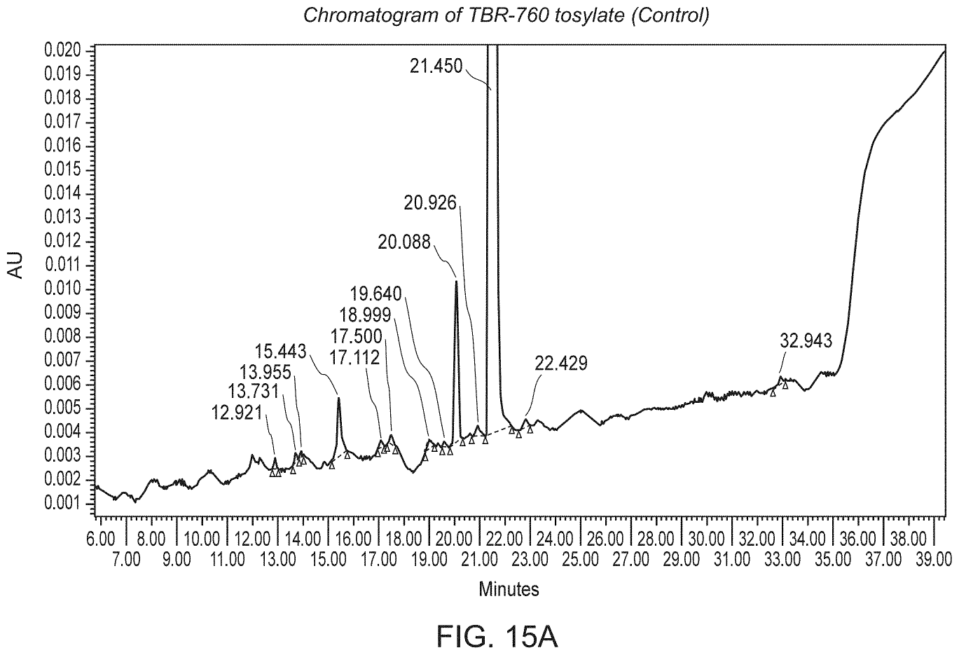

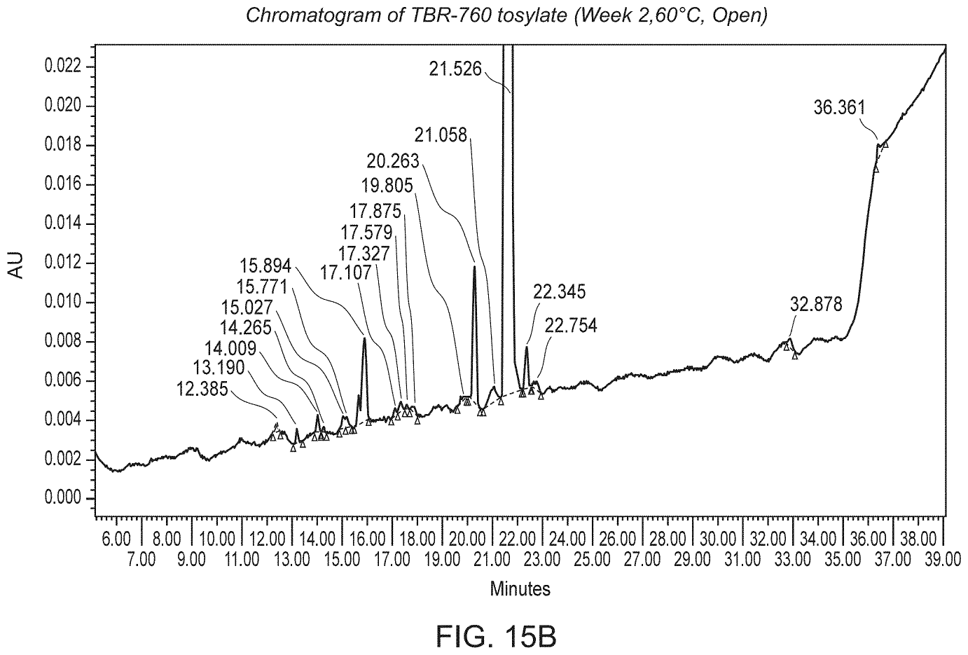

Data shown in individual chromatograms of each salt (FIGS. 11-15) show that after two weeks at 60° C. there are only two additional degradants of the chloride salt (comparing FIG. 12B to FIG. 12A), in contrast to the additional 17 degradants of the acetate salt following exposure to the same conditions (comparing FIG. 11A to 11B). Similarly, there are more than 10 additional degradants of the sulfate salt following exposure to 60° C. for two weeks (comparing chromatograms in FIGS. 13A and 13B) and at least an additional 4 degradants observed for both the mesylate salt (comparing FIGS. 14A and 14B) and the tosylate salt (comparing FIGS. 15A and 15B) exposed to the same conditions.

### Example 7: Photostability Studies of TBR-760 Salt Forms

In order to evaluate the light sensitivity and stability of TBR-760 chimeric peptide salts, all five salt forms were exposed to ultraviolet (UV) and visible light according to International Council for Harmonisation of Technical Requirements for Pharmaceuticals for Human Use (ICH) guideline Q1B for photostability testing. In brief, a Caron 6540-1 photostability chamber unit with separate UV and light chambers was used for the studies. Visible light exposure was 1.2 million lux hours and UV exposure was 200 watt hours/square meter.

Samples were placed in clear, sealed vials and stored in the appropriate chamber alongside dark controls in amber colored vials wrapped in aluminum foil. Control samples were stored in sealed amber vials at −20° C. The appearance of exposed samples was examined visually before being analyzed via UPLC. Results of the analyses for each exposed salt are summarized in Table 6.

TABLE 6
Results of Photostability Studies

| | | | | | Dark | Dark |
|---|---|---|---|---|---|---|
| Salt | | | Light- | UV- | Crtl- | Ctrl- |
| Form | Test | Control | exposed | exposed | Light | UV |
| Acetate | Appearance | white | yellowish | pale | white | white |
| | | | brown | yellowish | | |
| | | | | brown | | |
| | Purity | 98.35 | 92.14 | 96.16 | 98.34 | 98.42 |
| Chloride | Appearance | white | white | white | white | white |
| | Purity | 98.31 | 96.41 | 97.06 | 98.33 | 98.37 |
| Mesylate | Appearance | white | off-white | off-white | white | white |
| | Purity | 98.39 | 95.3 | 96.67 | 98.46 | 98.35 |
| Sulphate | Appearance | white | white | off-white | white | white |
| | Purity | 98.04 | 94.82 | 96.24 | 98.19 | 98.17 |
| Tosylate | Appearance | white | off-white | off-white | white | white |
| | Purity | 98.39 | 96.3 | 95.67 | 98.27 | 98.27 |

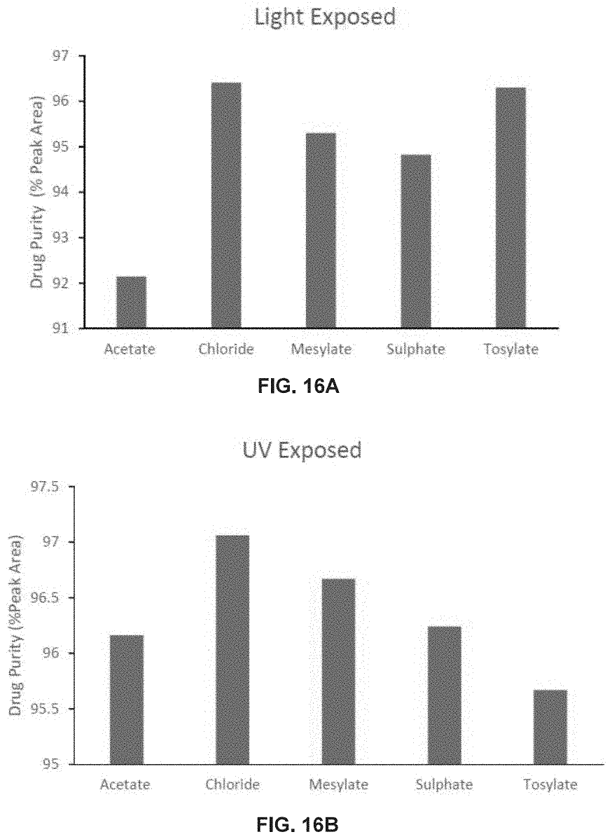

Results of the photostability studies demonstrate that the TBR-760 chloride and tosylate salts are comparably stable following light exposure, and significantly more stable than the TBR-760 acetate salt (FIG. 16A). The results further demonstrate that the TBR-760 chloride salt is more stable following UV exposure than the acetate, mesylate, sulfate, or tosylate salts (FIG. 16B).

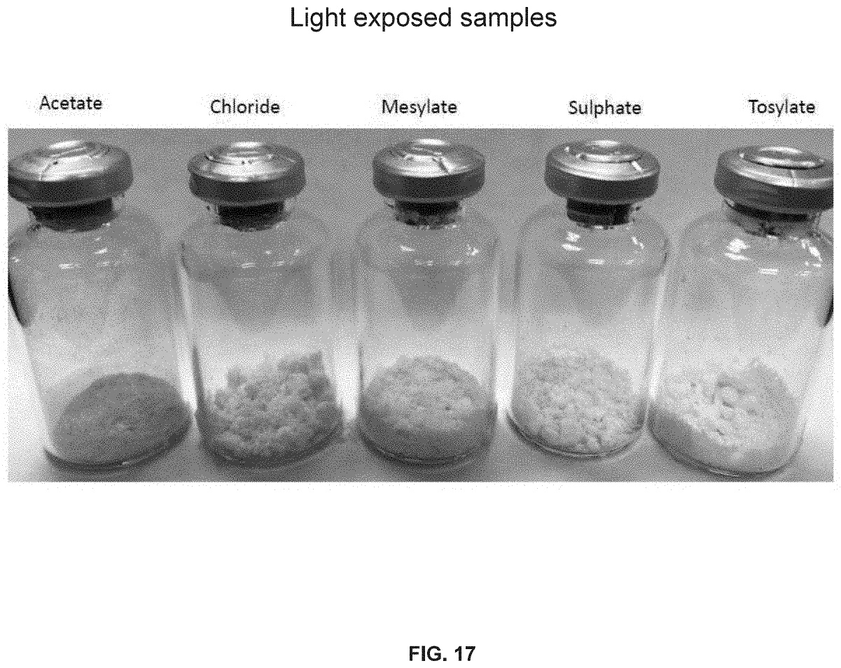

The TBR-760 acetate salt displayed noticeable visible discoloration from white to yellow-brown following light exposure (FIG. 17). The mesylate and tosylate salts displayed more subtle changes in appearance following light exposure as both appeared to discolor slightly from white to off-white (FIG. 17). The chloride and sulfate salt forms of TBR-760 showed no visible discoloration following light exposure (FIG. 17).

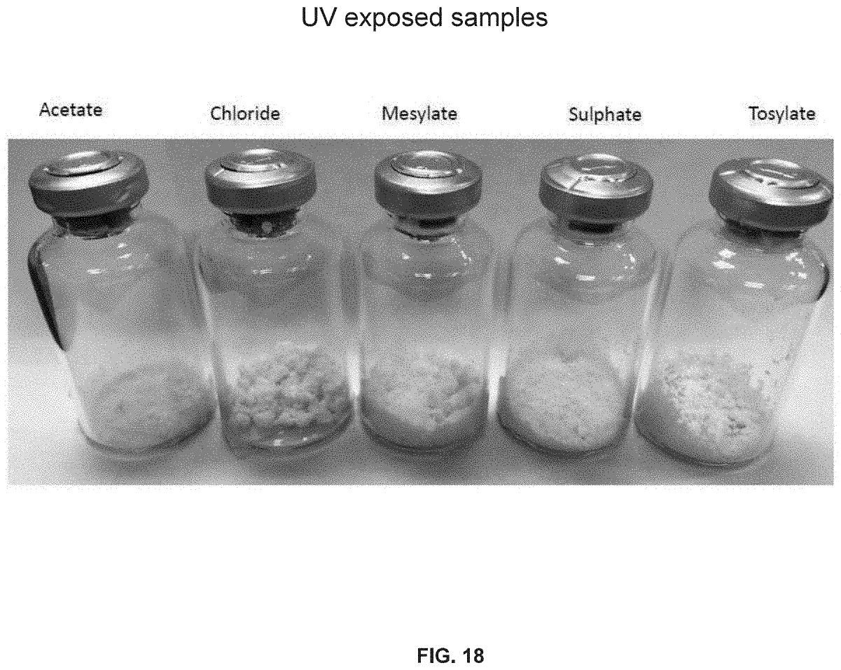

Similarly, the TBR-760 acetate salt displayed discoloration from white to a pale yellow-brown color following exposure to UV light (FIG. 18). The mesylate, tosylate, and sulfate salts showed more subtle discoloration from white to off-white following UV exposure (FIG. 18). The appearance of the chloride salt remained unchanged following UV exposure.

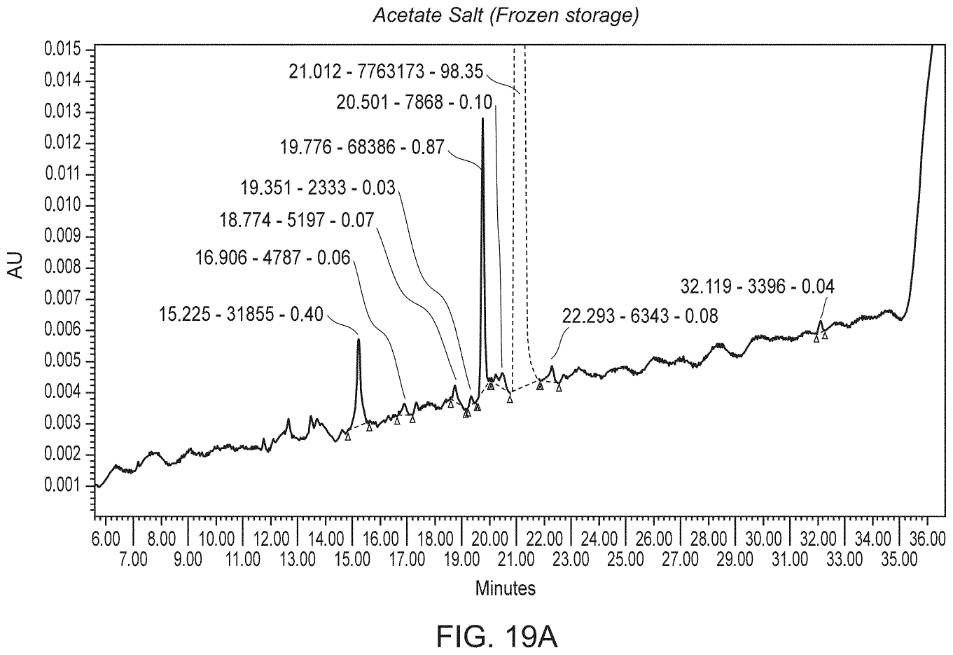

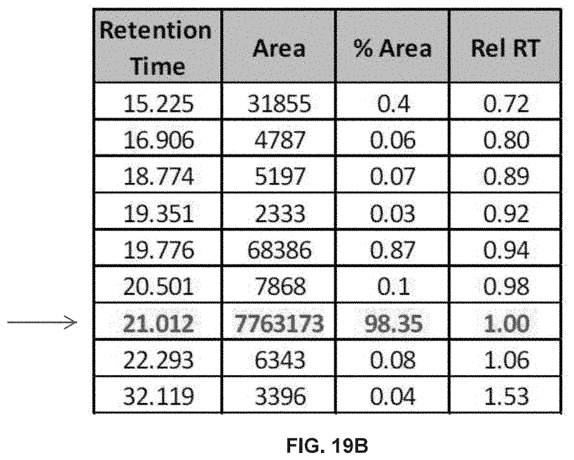

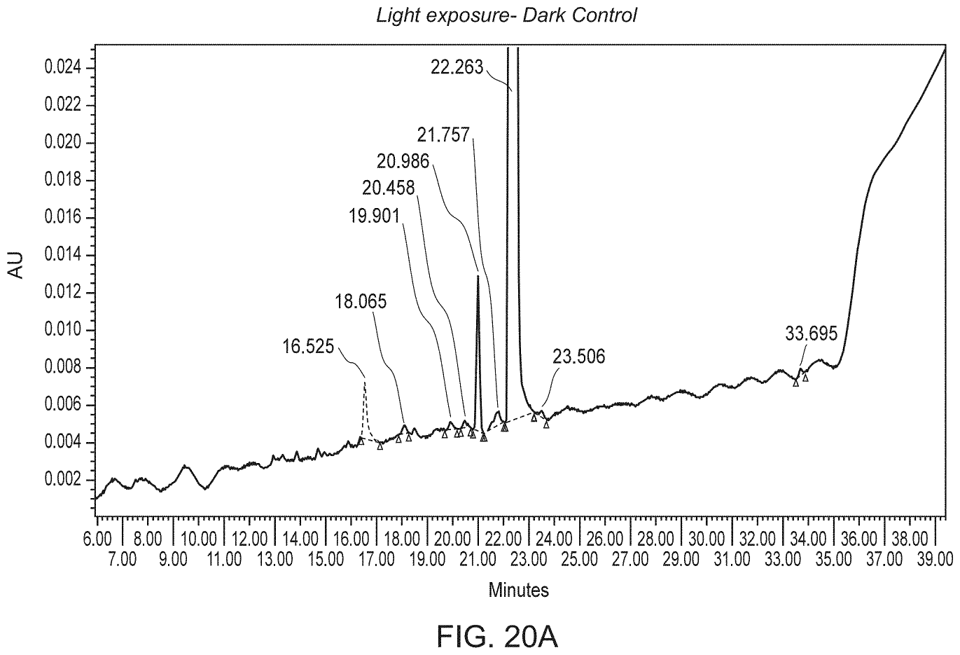

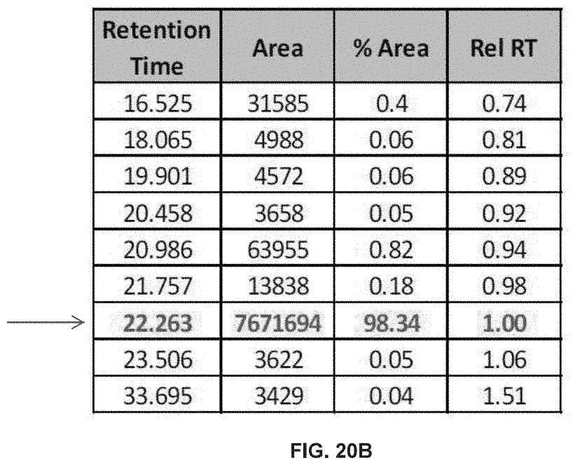

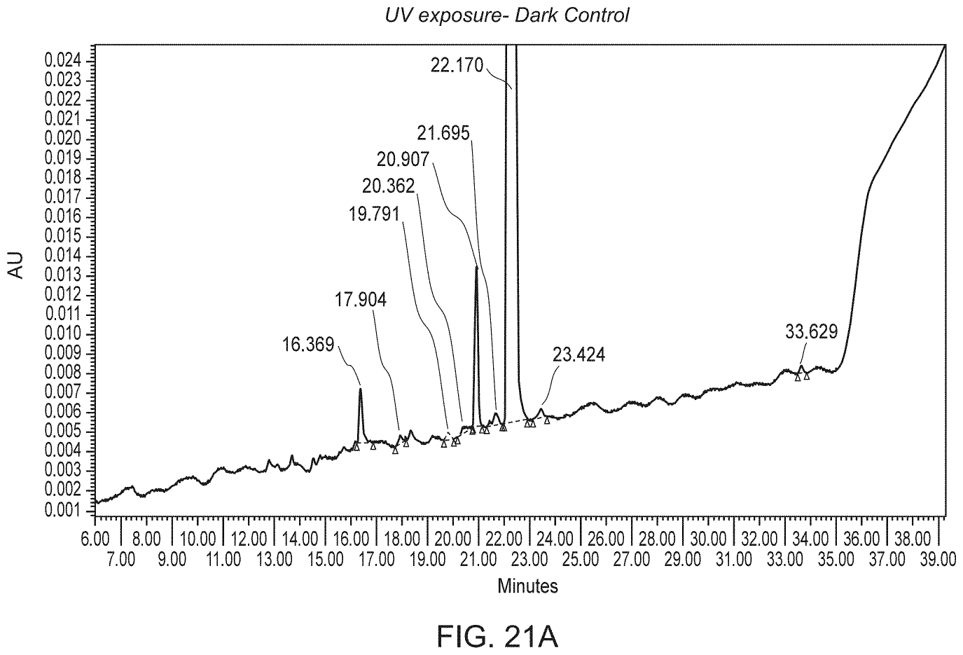

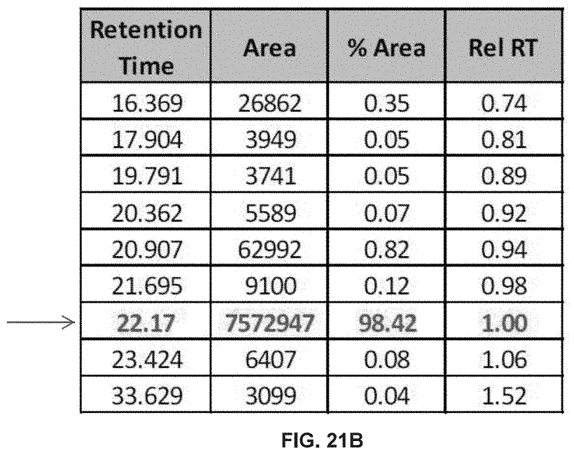

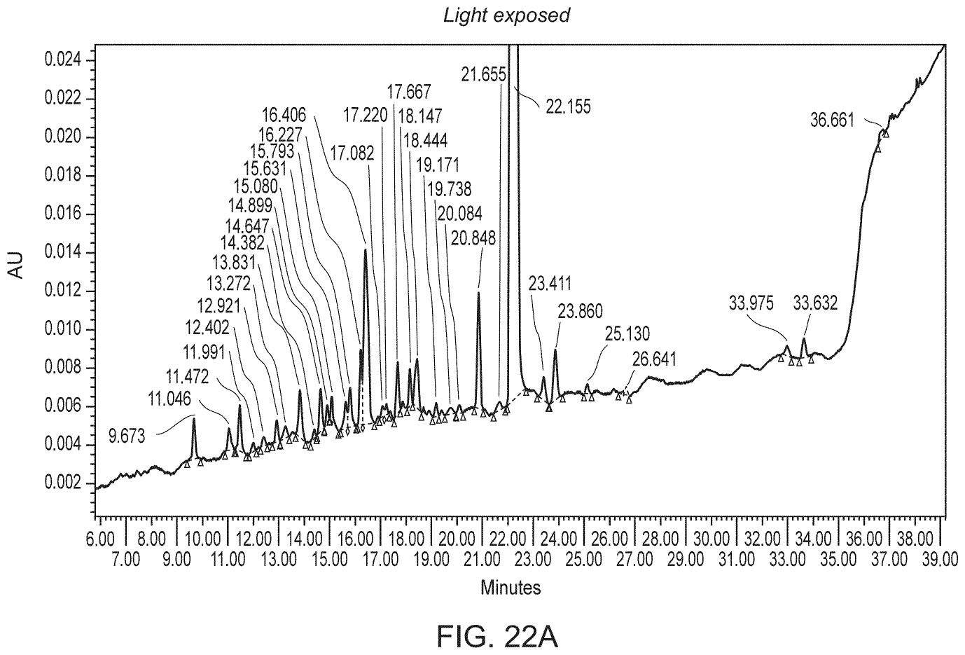

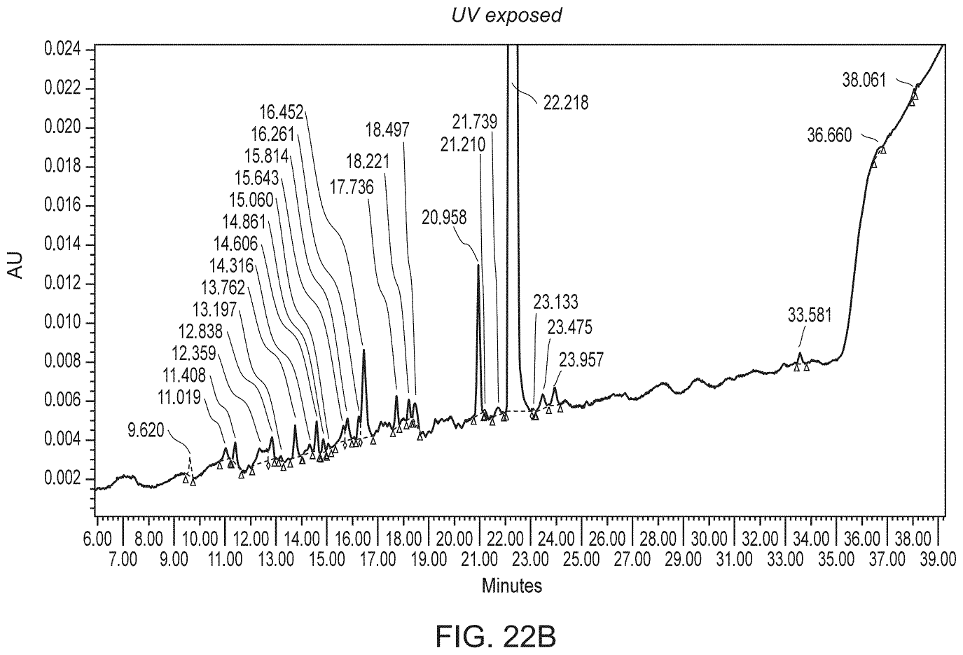

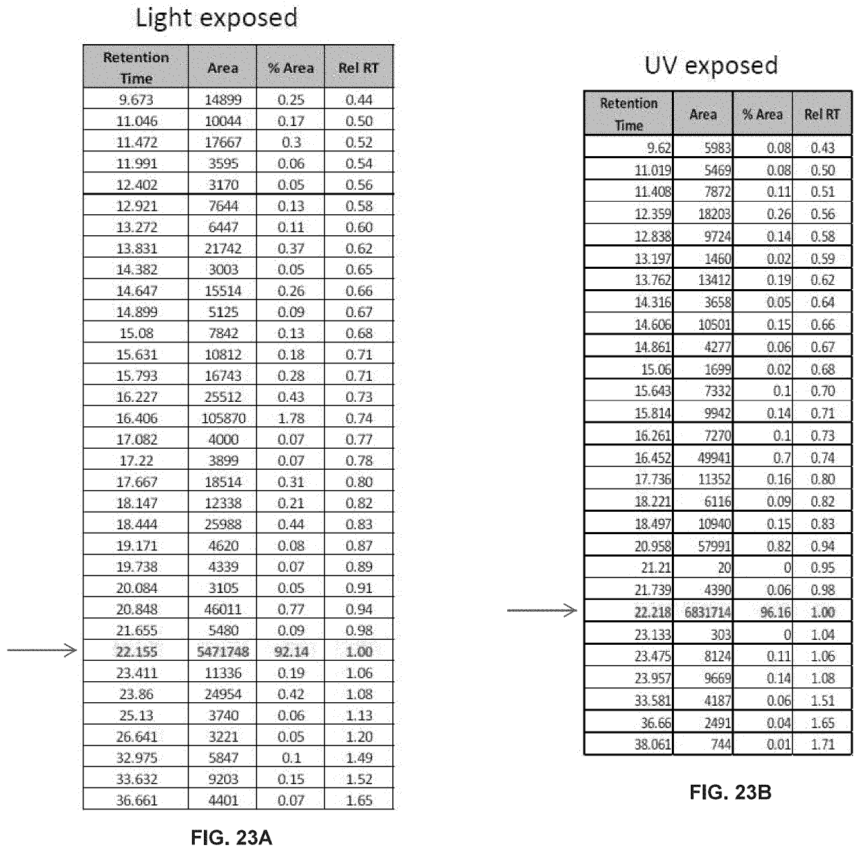

Results of UPLC analysis of the controls and samples exposed to visible and UV light demonstrate that the TBR-760 acetate salt does not degrade under dark conditions (compare results of FIGS. 20A-21B with FIGS. 19A and 19B). However, the TBR-760 acetate is degraded following exposure to visible light as well as UV light as shown by the large number of degradant peaks following UPLC analysis of the exposed samples (see FIGS. 22A-22B and FIGS. 23A-23B).

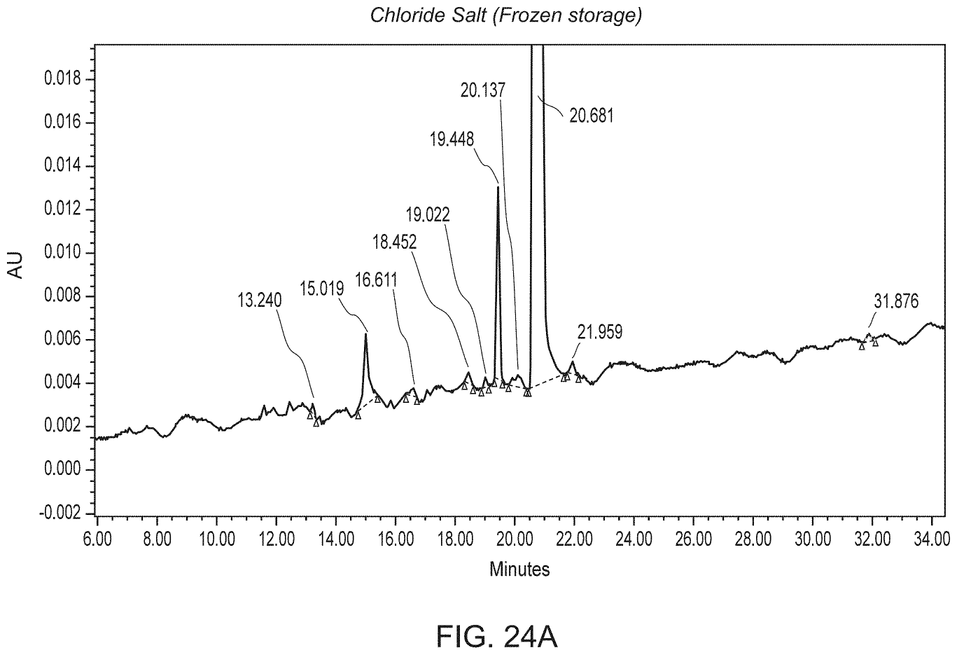

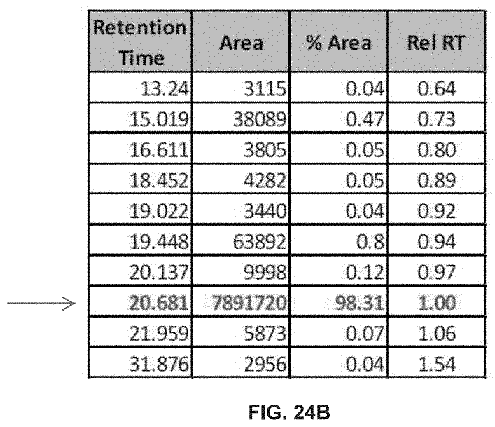

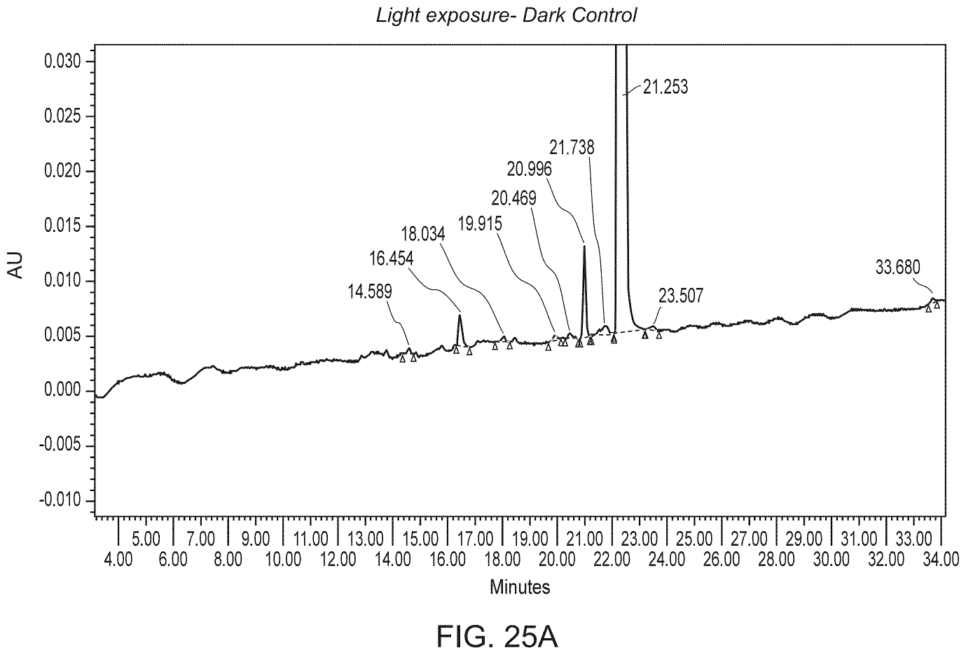

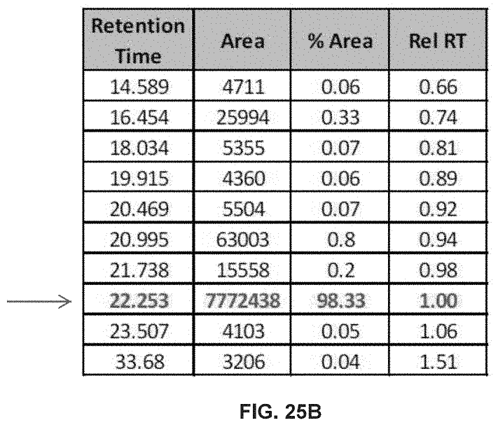

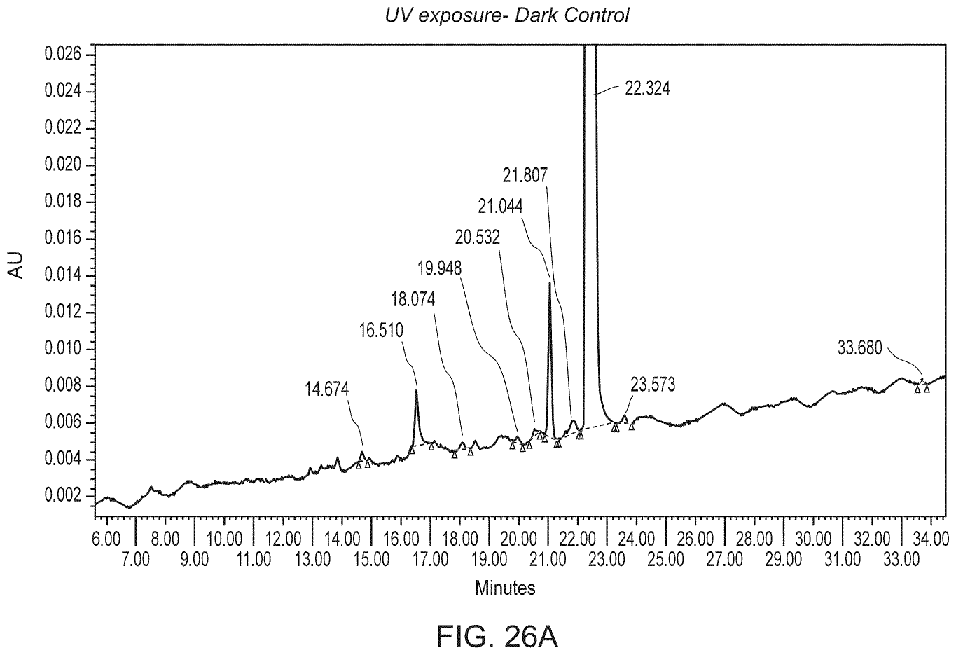

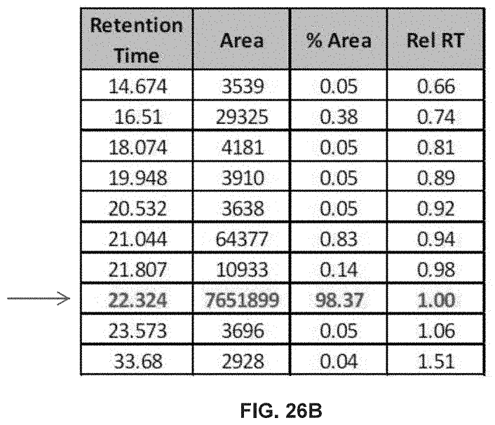

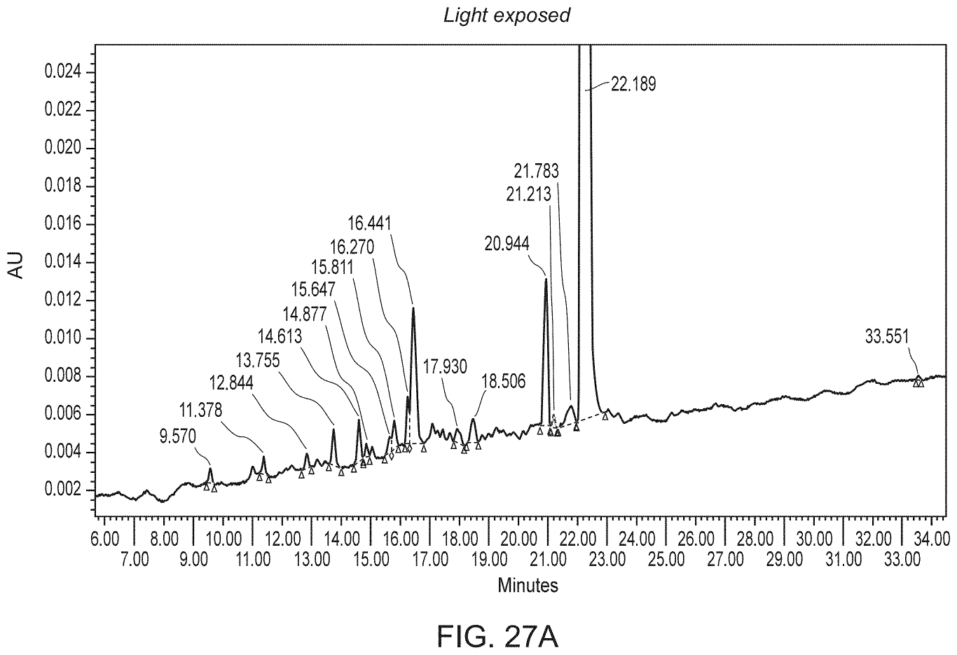

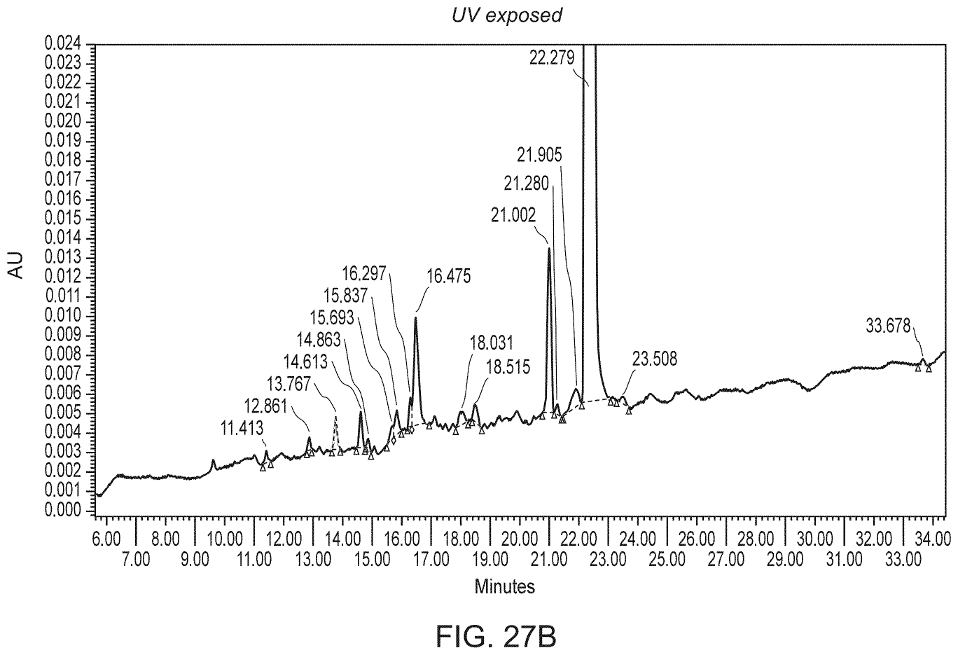

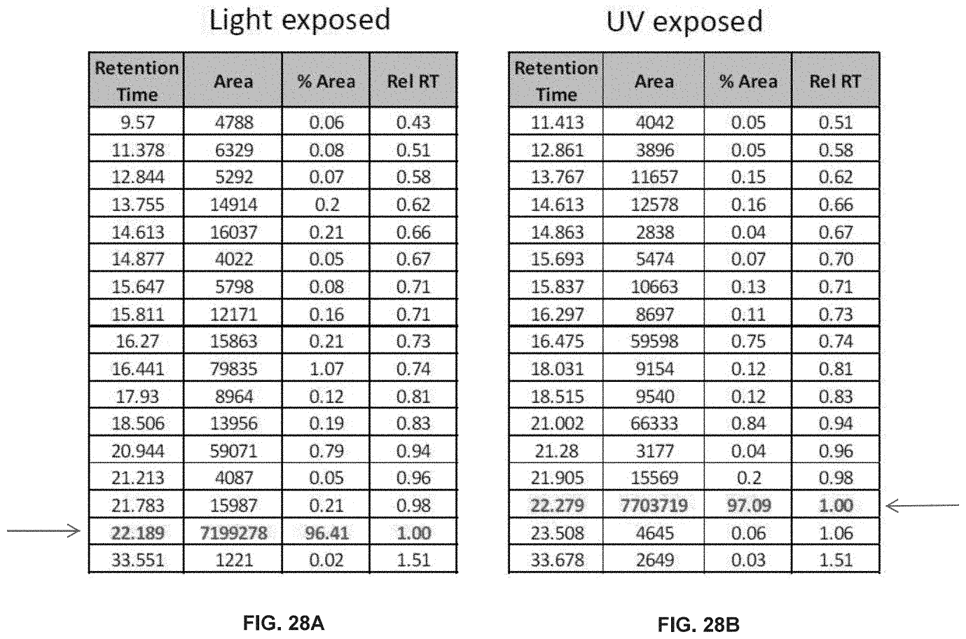

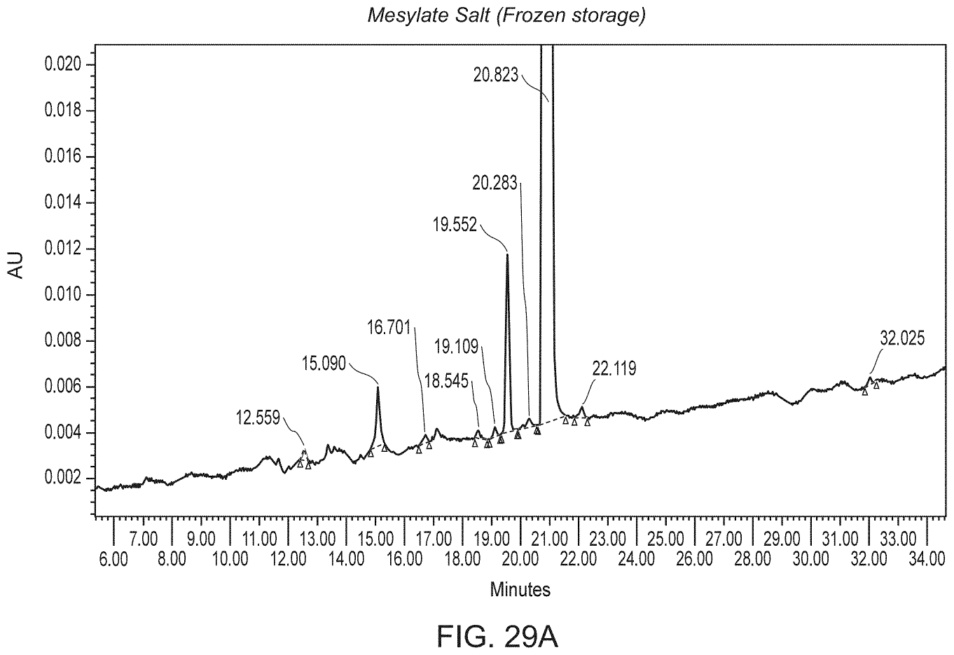

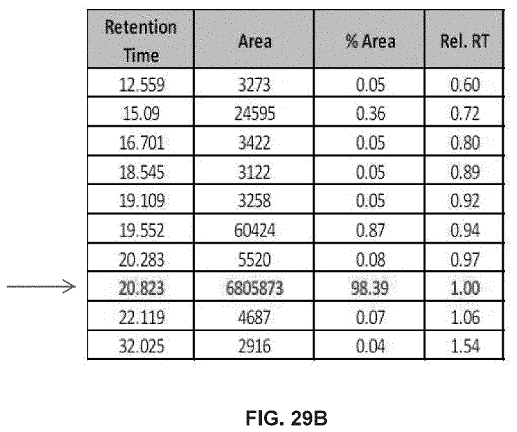

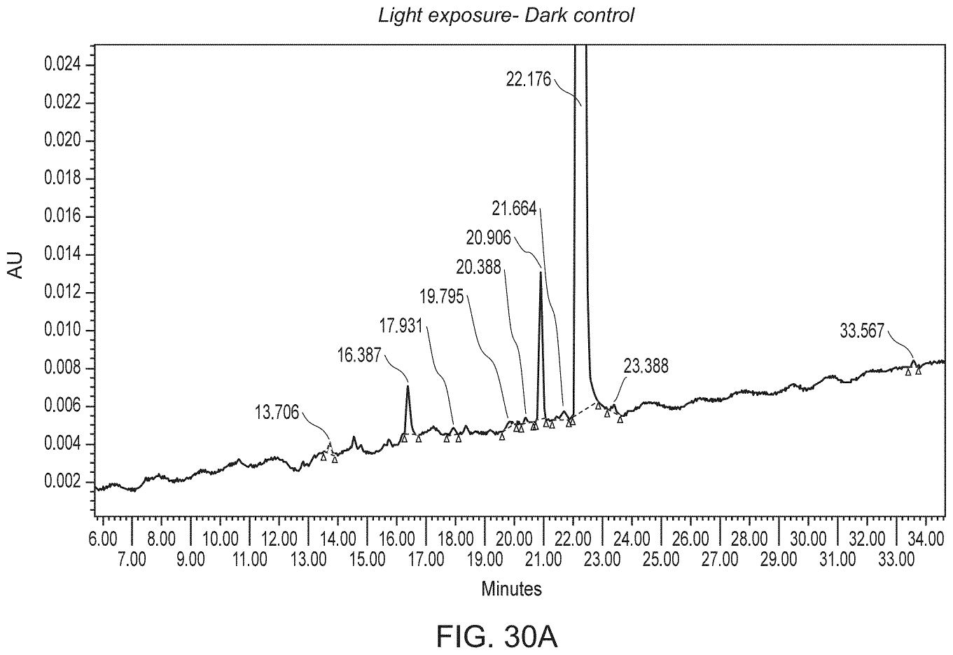

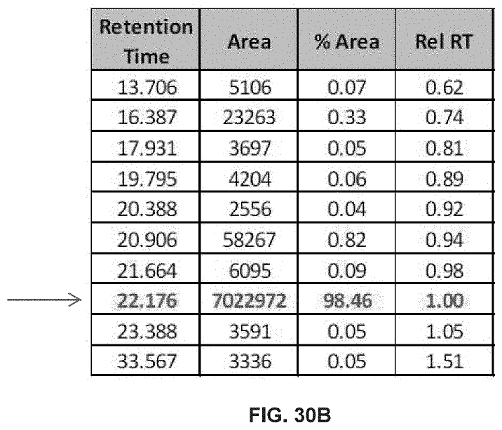

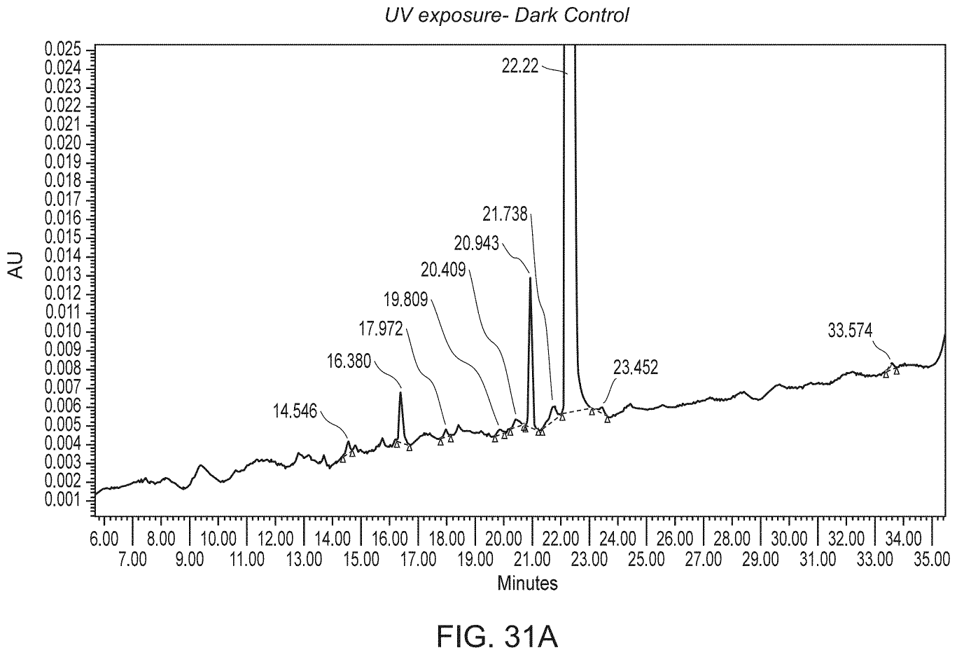

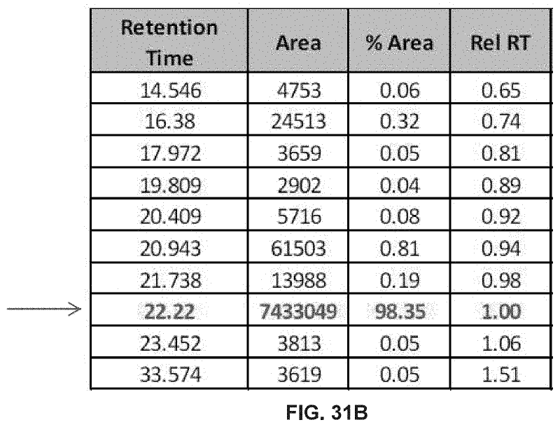

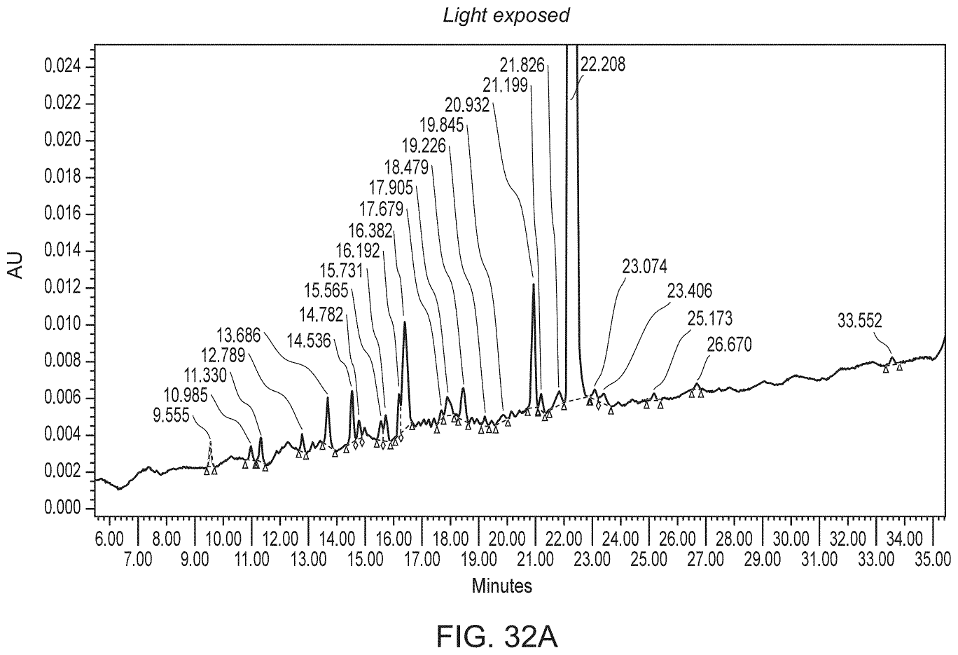

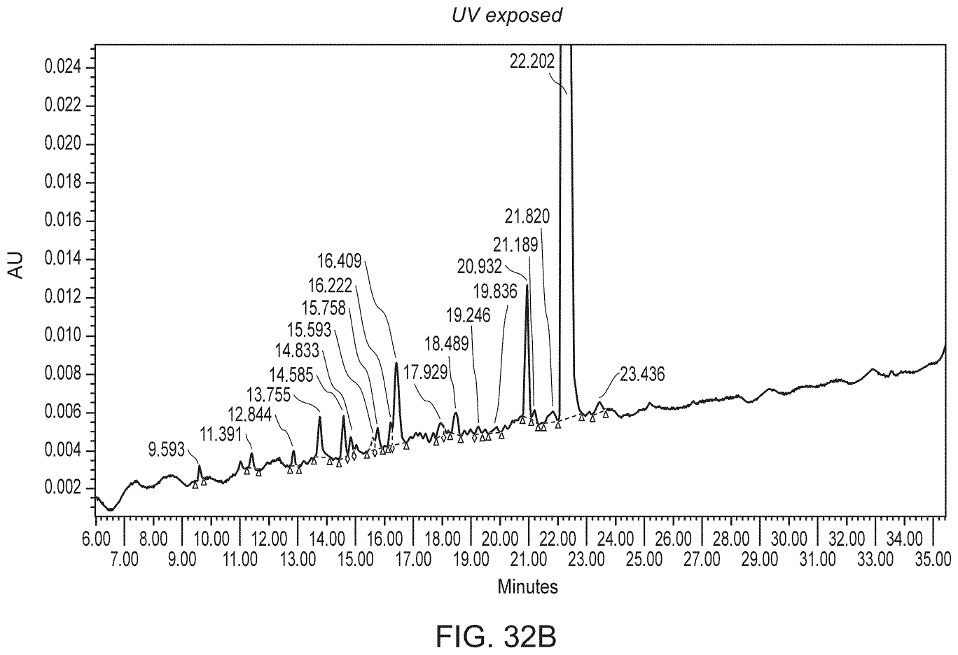

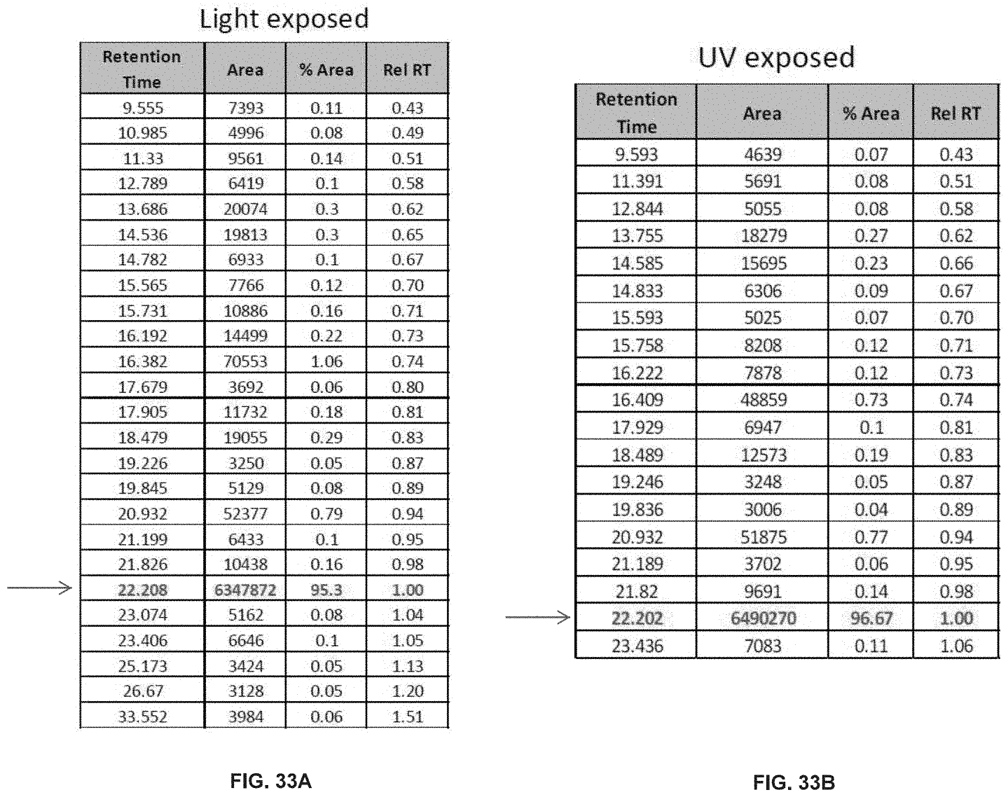

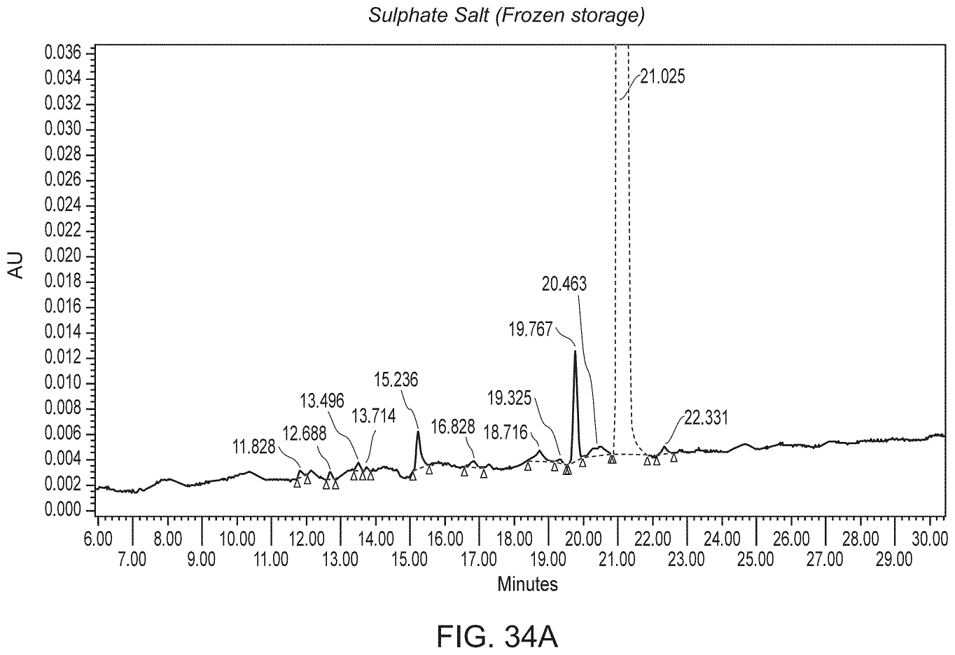

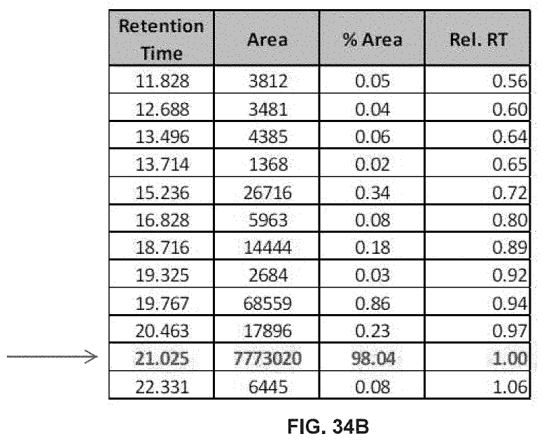

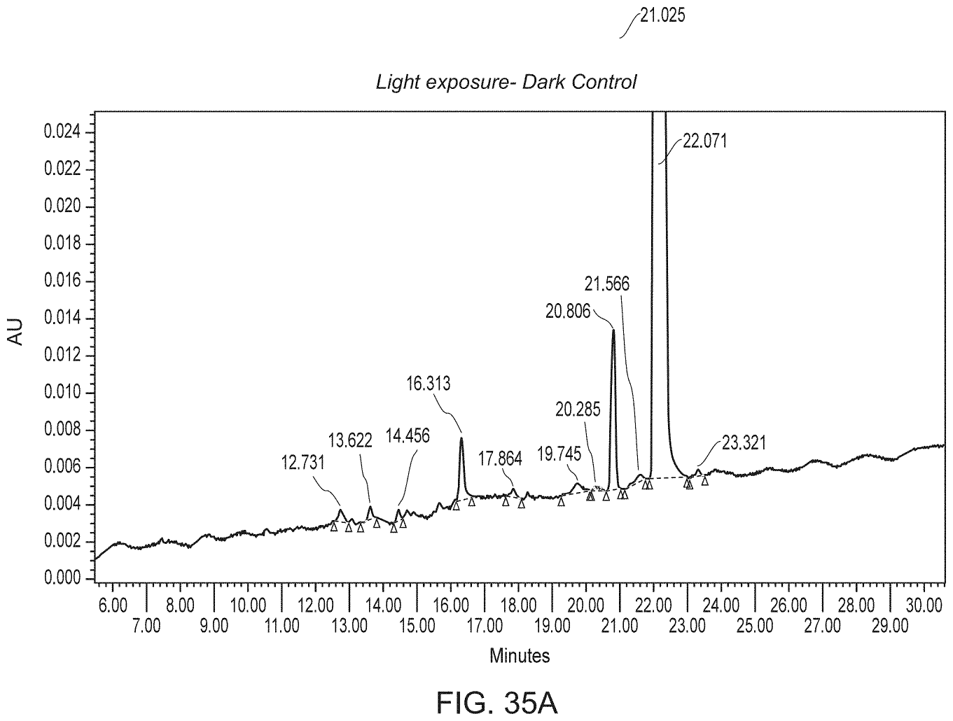

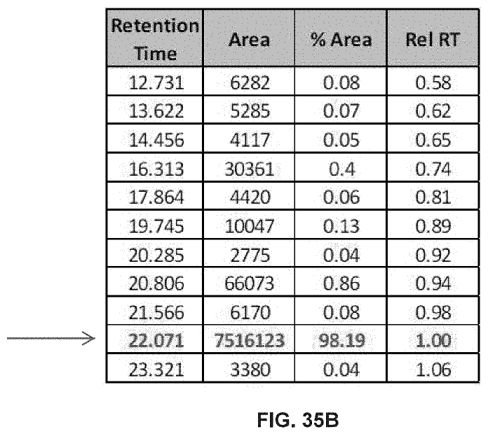

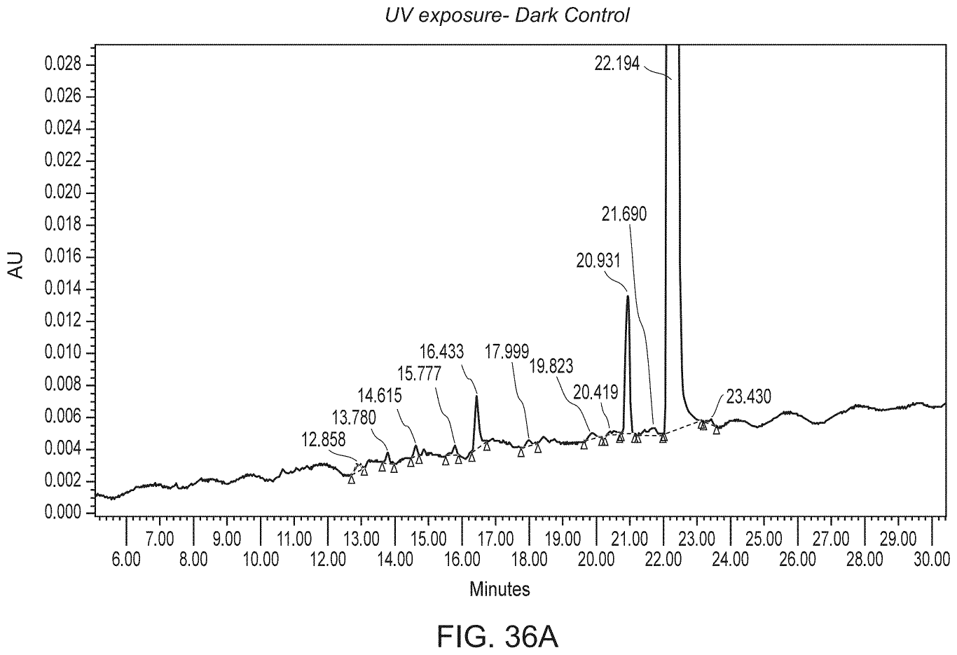

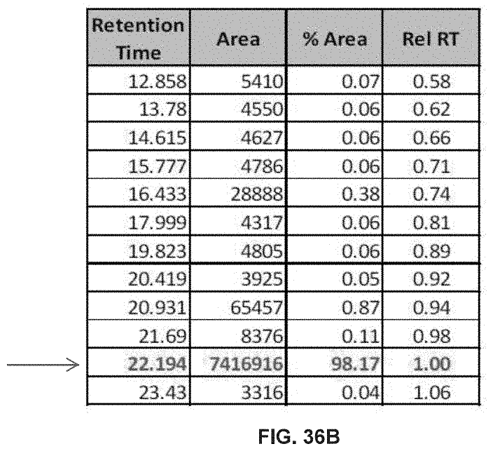

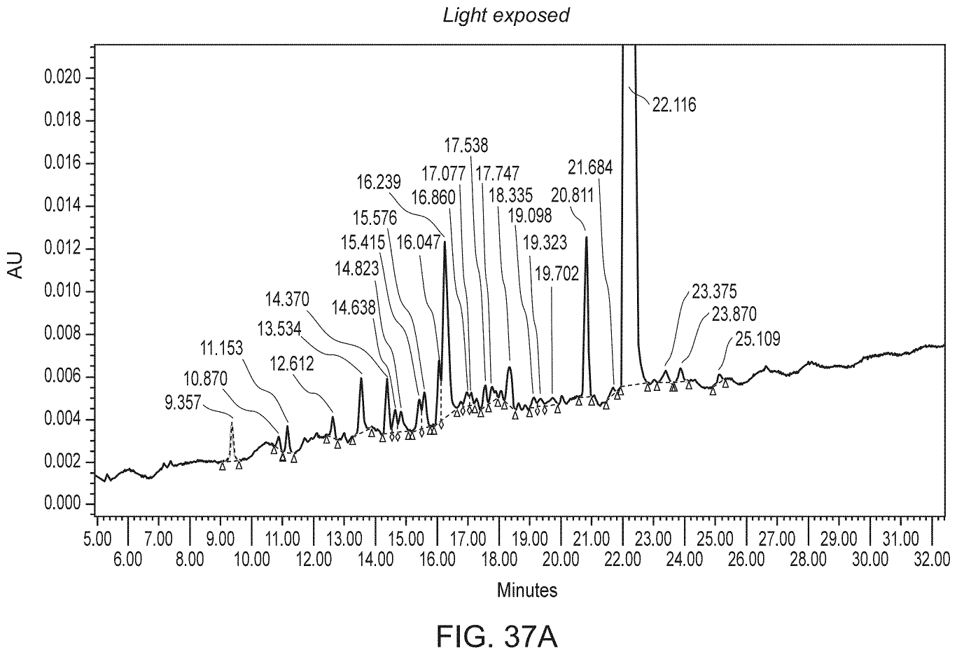

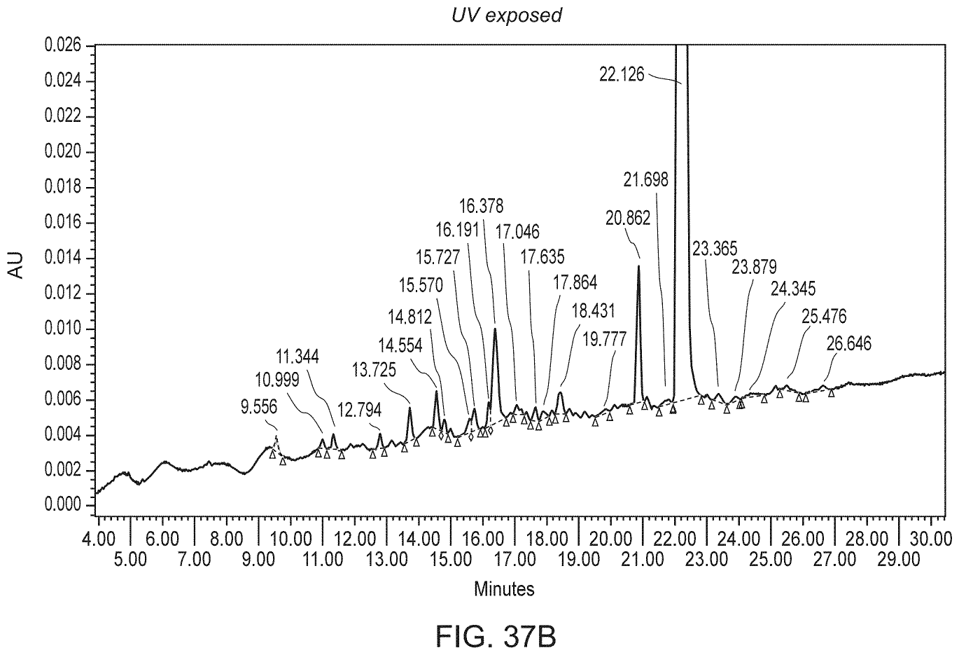

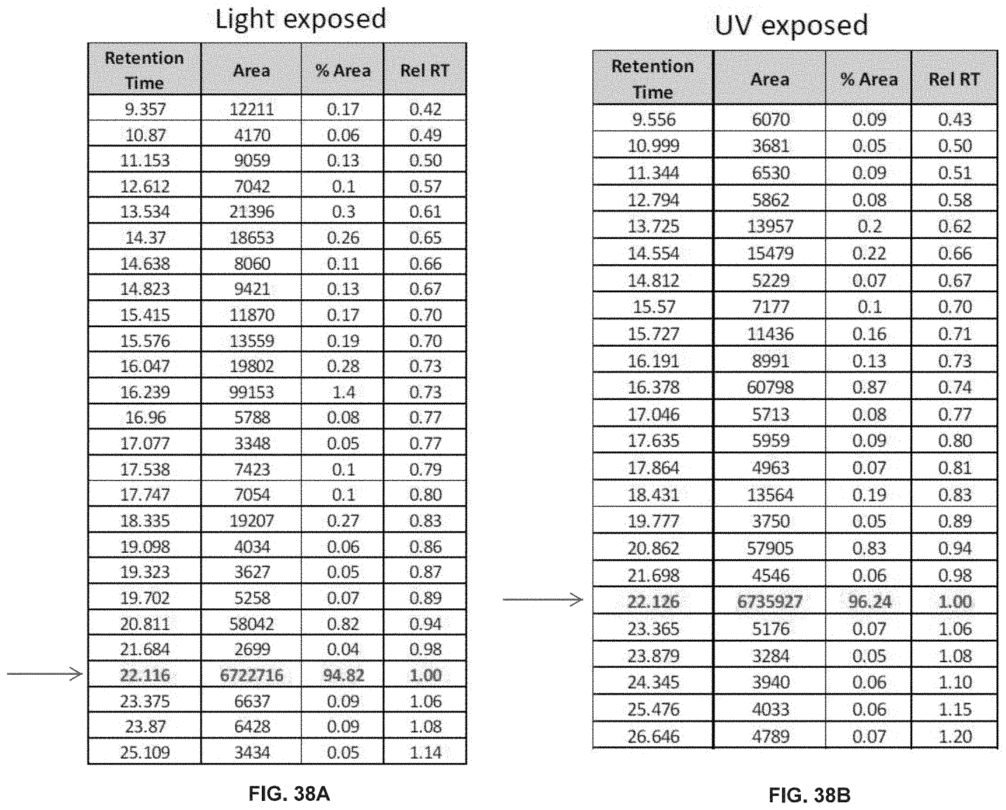

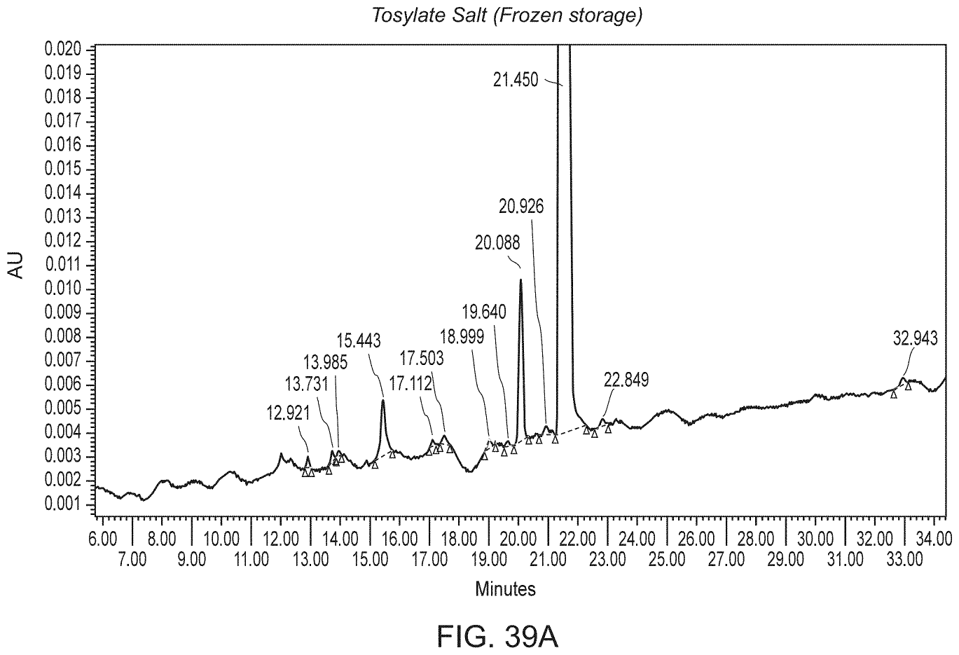

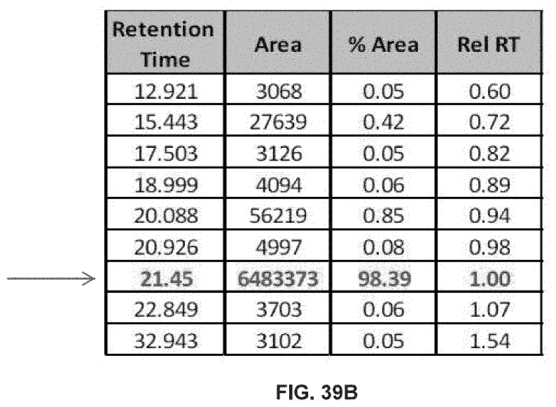

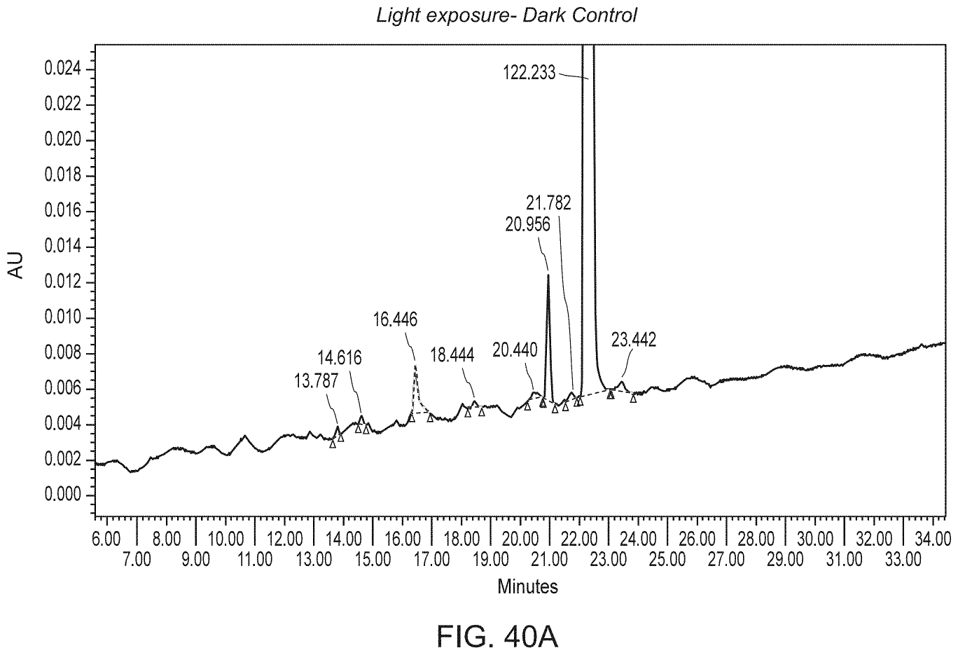

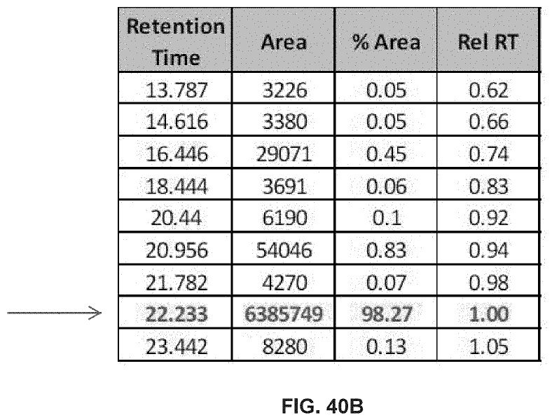

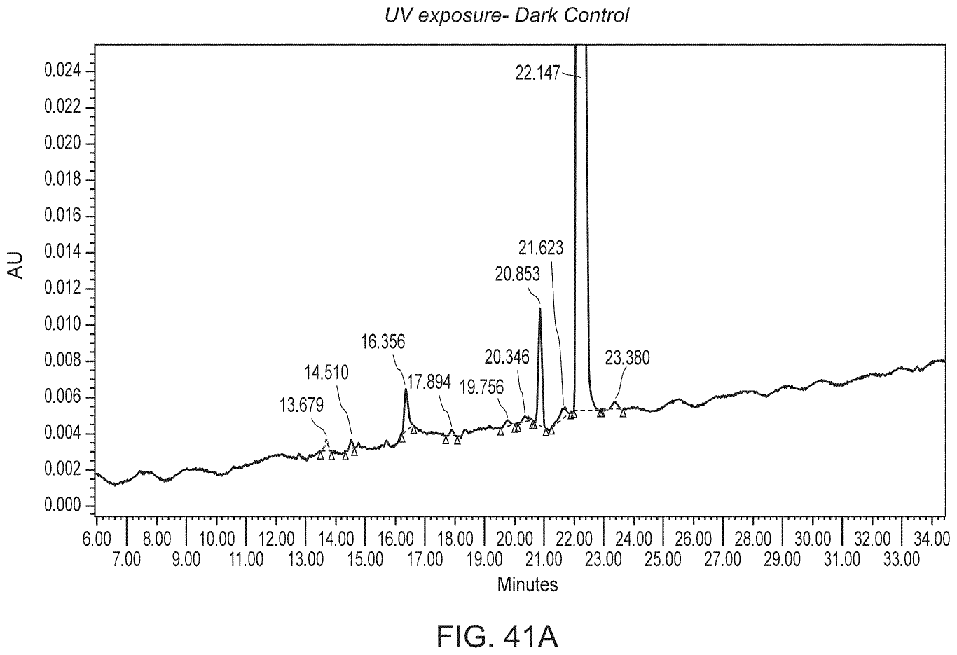

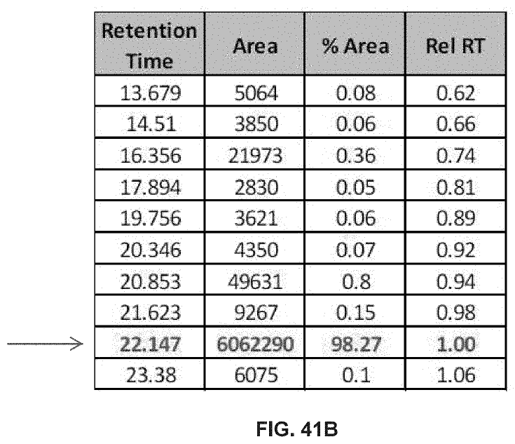

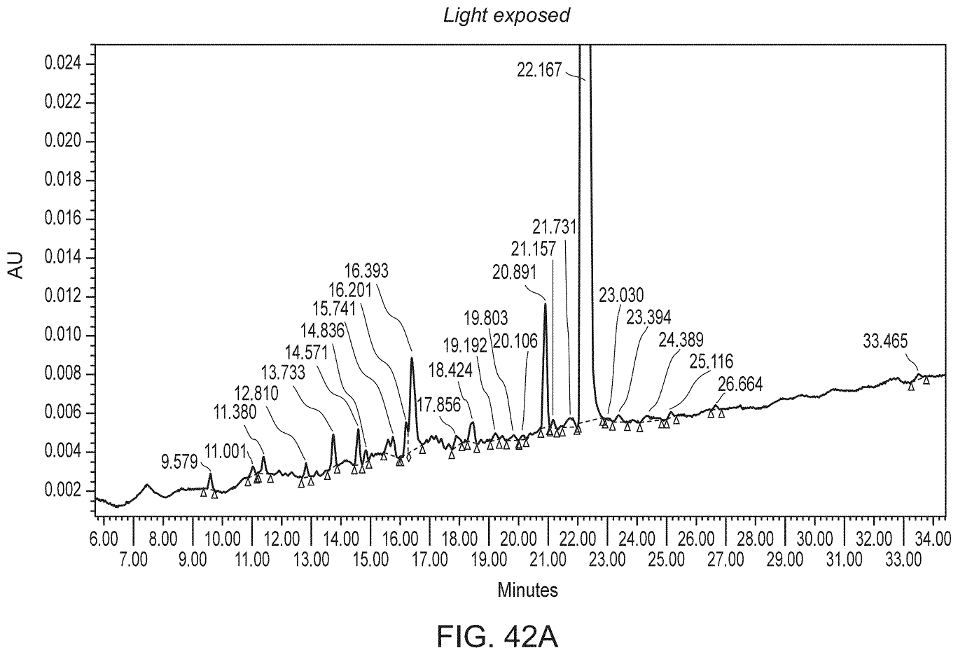

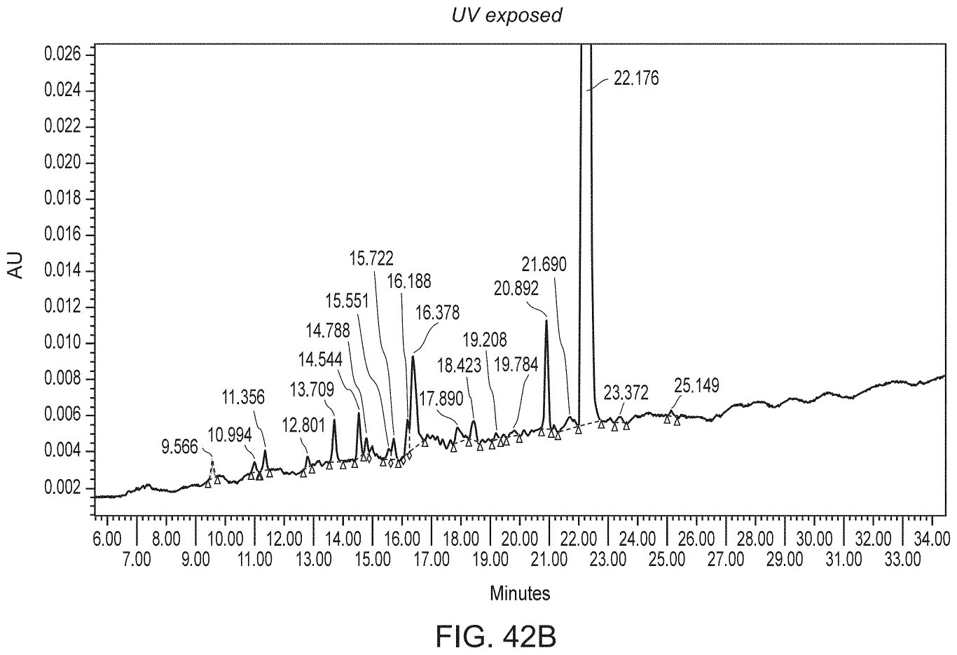

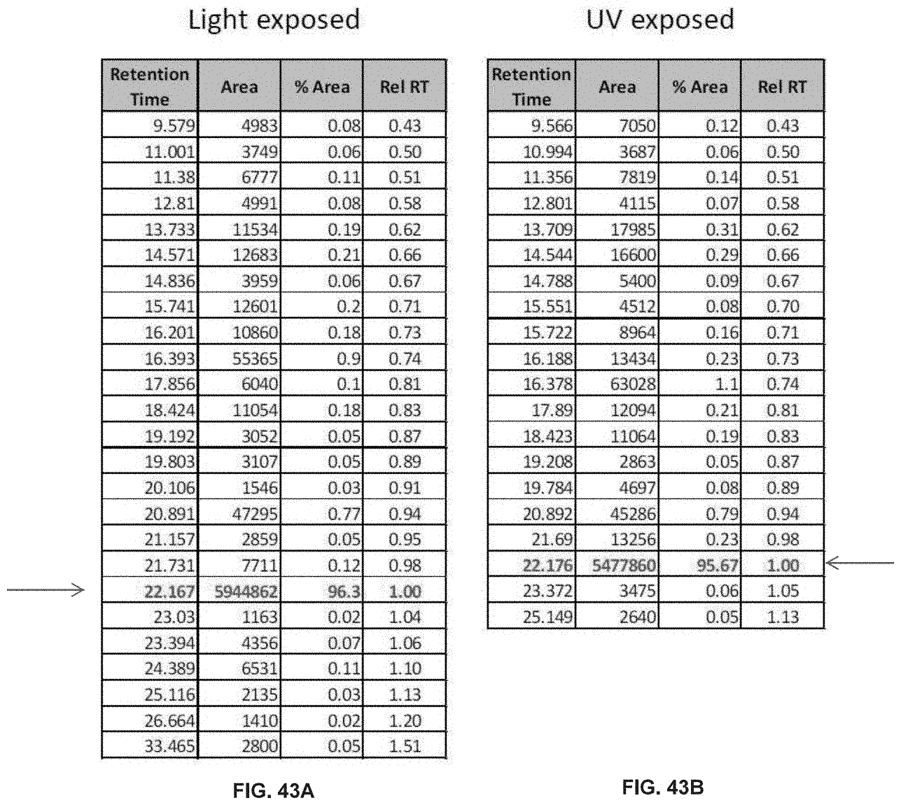

Similarly, UPLC analyses show that the other four TBR-760 salt forms tested also do not degrade under dark conditions. (See FIGS. 25A-25B and 26A-26B compared to FIGS. 24A-24B (chloride salt); FIGS. 30A-30B and 31A-31B compared to FIGS. 29A-29B (mesylate salt); FIGS. 35A-35B and 36A-36B compared to FIGS. 34A-34B (sulfate salt); and FIGS. 40A-40B and 41A-41B compared to FIGS. 39A-39B (tosylate salt).) The results show that there are additional degradants of each salt form following exposure to visible and UV light. (See FIGS. 27A-27B and 28A-28B (chloride salt); FIGS. 32A-32B and 33A-33B (mesylate salt); FIGS. 37A-37B and 38A-38B (sulphate salt); and FIGS. 42A-42B and 43A-43B (tosylate salt).

The UPLC results show that upon exposure to either visible or UV light, TBR-760 chloride (FIGS. 28A-28B), TBR-760 mesylate (FIGS. 33A-33B), TBR-760 sulfate (FIGS. 38A-38B), and TBR-760 tosylate (FIGS. 43A-43B) are more resistant to degradation than TBR-760 acetate (FIGS. 23A-23B). Based on these results, the chloride and mesylate salt forms of TBR-760 appear to be similarly resistant to degradation following exposure to either visible or UV light.

Together, results of these studies demonstrate that the TBR-760 chloride salt is more stable following exposure to both visible and UV light than the TBR-760 acetate, mesylate, sulfate, and tosylate salt forms.

### Example 8: Thermostability of Alternative Salt Forms of Lanreotide and TBR-760

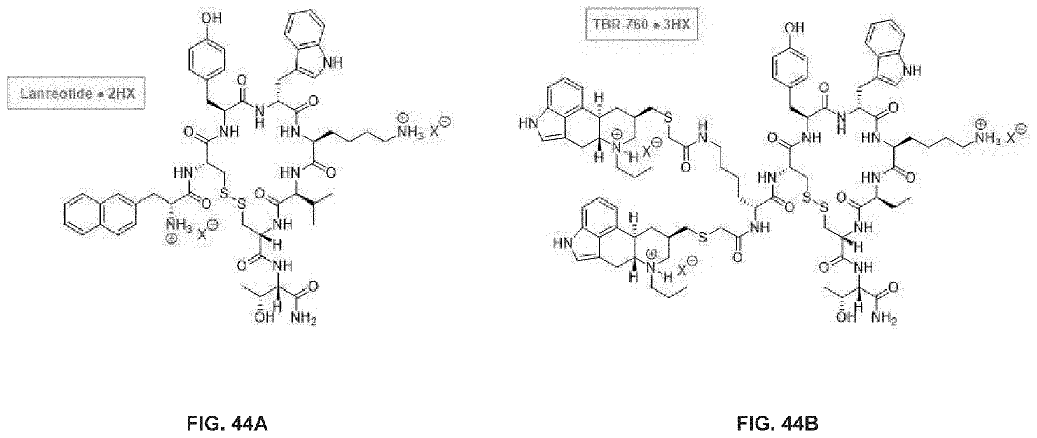

TBR-760 is a chimeric somatostatin (SST)-dopamine (DA) compound with potent agonist activity at both SST type 2 and DA type 2 receptors. The structure of TBR-760 is shown in FIG. 44B. Both the macrocyclic peptide moiety and the Dop2 cyclic group would be expected to contribute to TBR-760's physicochemical properties. In prior pre-clinical and clinical studies, TBR-760 was administered as the acetate salt. Acetate is the most commonly used salt form for peptide therapeutics.

Lanreotide is a somatostatin analog approved by FDA to slow the growth of gastrointestinal and pancreatic neuroendocrine tumors. Lanreotide shares major structural features with TBR-760—the structures of lanreotide and TBR-760 are compared in FIGS. 44A and 44B. Lanreotide is approved and sold as the acetate salt.

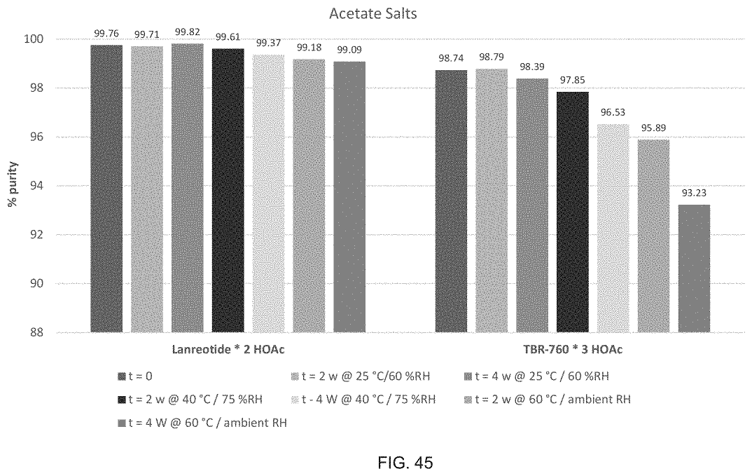

We conducted thermostability studies of lanreotide acetate substantially as described in Example 6. These experiments confirmed that lanreotide acetate is stable when exposed to increasing temperature and humidity conditions over time (FIG. 45). Surprisingly, however, the stability of lanreotide acetate did not predict stability of TBR-760 acetate: thermostability studies of TBR-760 acetate demonstrated that TBR-760 acetate does not maintain stability under the same conditions, instead showing greater than 5% degradation following 4 weeks at 60° C. (FIG. 45). This degree of thermal and humidity instability is disfavored for a pharmaceutical agent, particularly one intended for patient self-administration at home.

To determine whether a thermostable salt form of TBR-760 could be manufactured, a salt exchange was performed as described in Example 1 to generate bromide and chloride salts of TBR-760. Lanreotide bromide and lanreotide chloride salts were also generated and assessed for thermostability to evaluate the effect of the salt form on compound stability and to compare stability of lanreotide and TBR-760 of each salt.

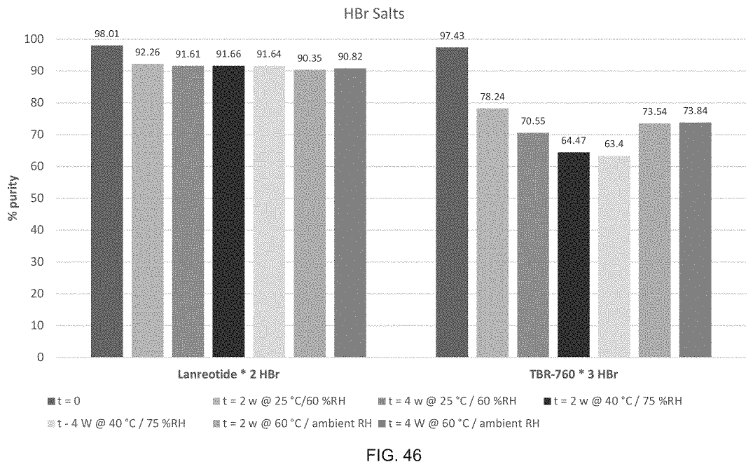

As shown in FIG. 46, lanreotide hydrobromide was less stable than lanreotide acetate with more than 7% of lanreotide bromide degraded following 4 weeks at 60° C. and ambient humidity. TBR-760 was even less stable, with more than 23% of TBR-760 bromide degraded under the same conditions (FIG. 46).

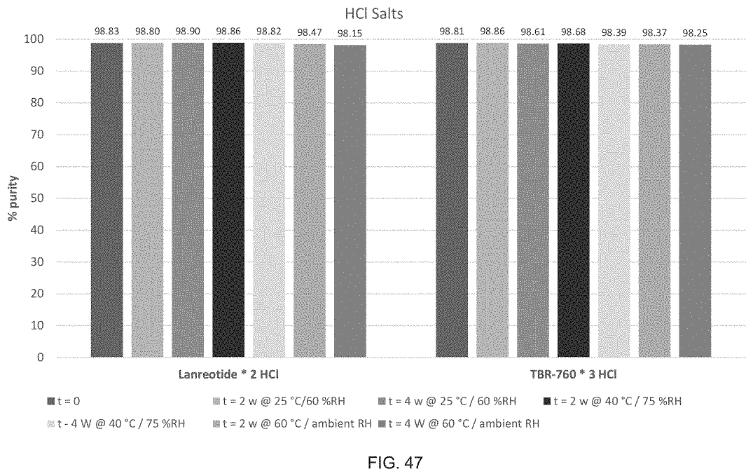

In contrast, the chloride salt form of TBR-760 (FIG. 47) was stable, with less than 0.6% of TBR-760 chloride degraded following 4 weeks at 60° C. and ambient relative humidity.

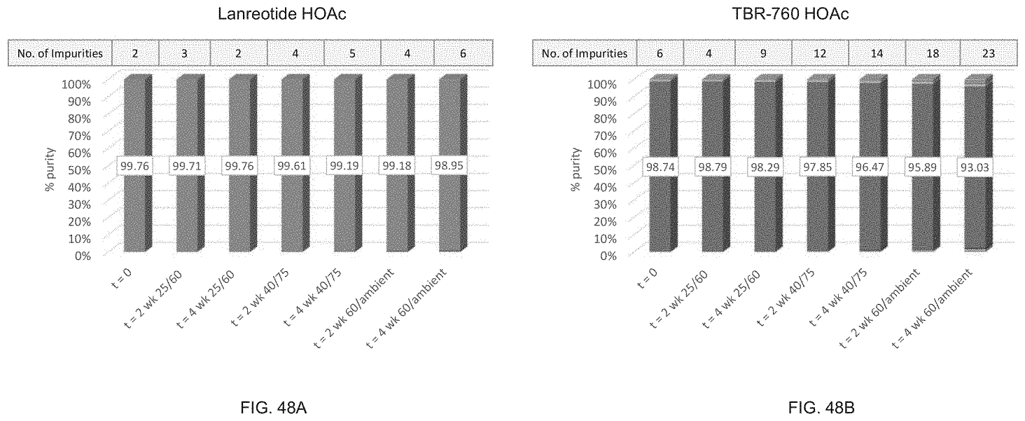

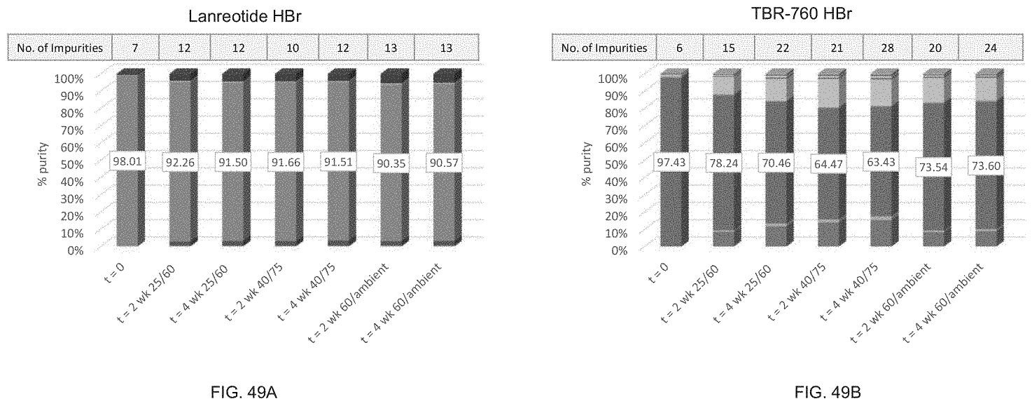

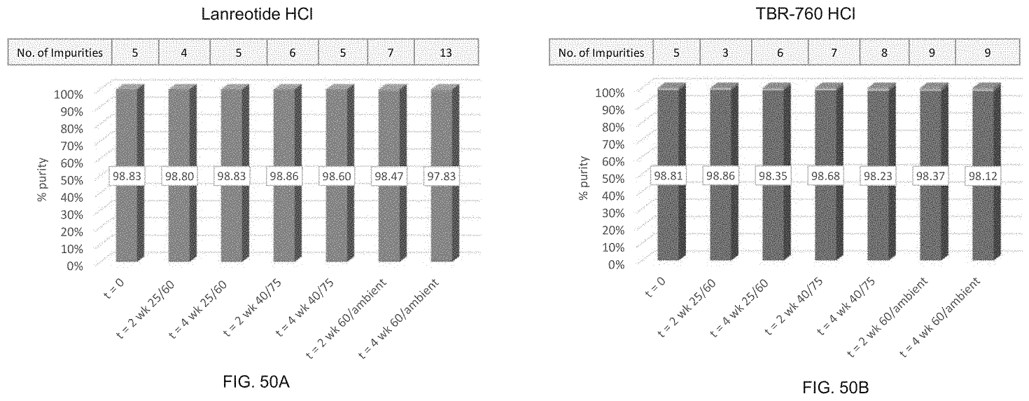

UPLC assays performed as described in Example 6 were further used to determine the number of degradants—and thus the number of impurities—in each sample. Results showing the purity assessment of each sample following thermostability studies are shown in FIGS. 48A-48B (lanreotide and TBR-760 acetate salts), 49A-49B (lanreotide and TBR-760 bromide salts), and 50A-50B (lanreotide and TBR-760 chloride salts). The results demonstrate that the TBR-760 chloride samples have a higher percent purity of TBR-760 under all conditions compared to samples of TBR-760 acetate and TBR-760 bromide (comparing FIG. 50B to FIGS. 48B and 49B). Consistent with the higher percent purity, there were fewer impurities found in the TBR-760 chloride samples from all conditions compared to the TBR-760 acetate and TBR-760 bromide samples.

### Example 9: Photostability of Alternative Salt Forms of Lanreotide and TBR-760

Photostability of lanreotide acetate, lanreotide bromide, lanreotide chloride, TBR-760 acetate, TBR-760 bromide and TBR760 chloride was tested according to the methods of Example 7. Four samples were prepared for each salt form: one control for each of the UV and visible light exposure conditions (controls isolated from light) and one sample for exposure to each of the UV and visible light conditions. For each sample, 2-5 mg of the salt was weighed into 3 mL clear serum vials and stoppered. Vials containing control samples were wrapped in aluminum foil.

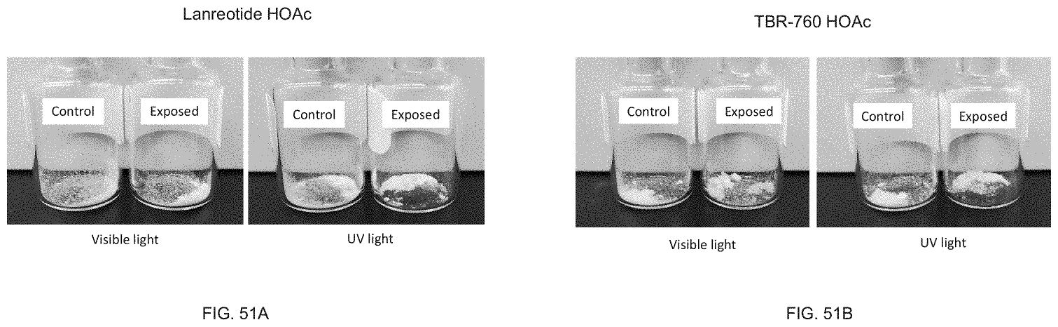

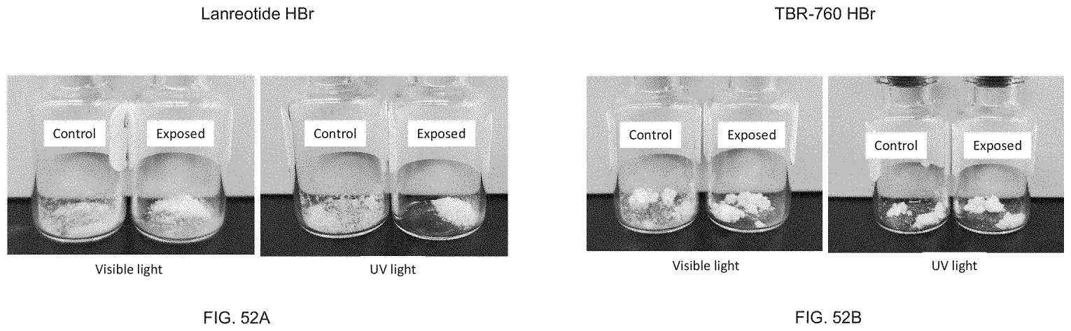

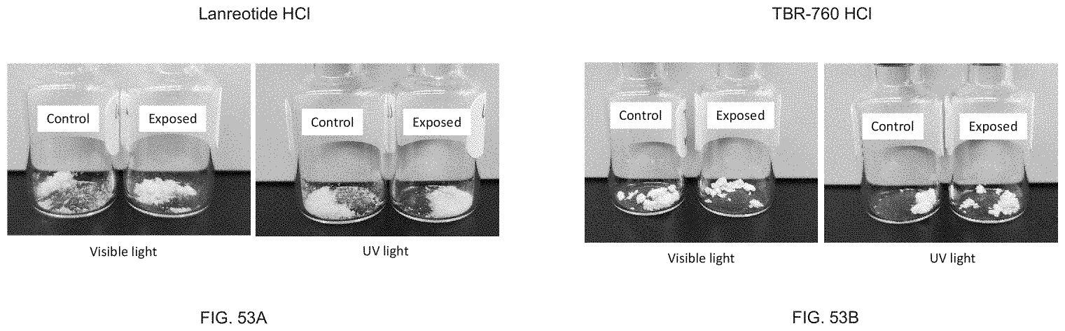

Results in FIGS. 51A, 52A, and 53A show that the visible appearance of all three salt forms of lanreotide remained unchanged following exposure to visible and UV light when compared to the respective controls. Of the TBR-760 salt forms evaluated, only TBR-760 acetate changed appearance following exposure to visible and UV light (FIG. 51B). Following exposure to both visible and UV light, TBR-760 acetate visibly changed color from a white salt (control) to a yellow/brown salt (both visible and UV light) (FIG. 51B). In contrast, the TBR-760 bromide and TBR-760 chloride salts did not visibly change following exposure to either visible or UV light (FIGS. 52B and 53B).

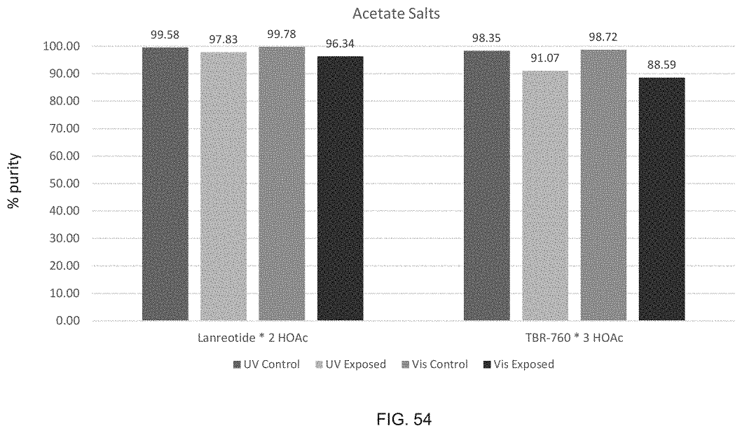

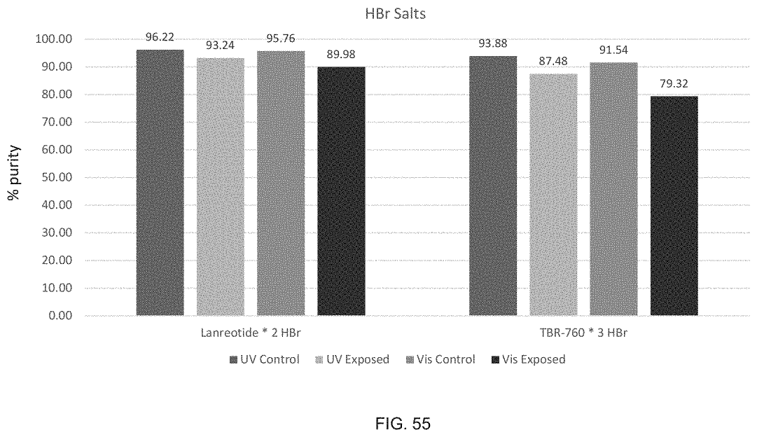

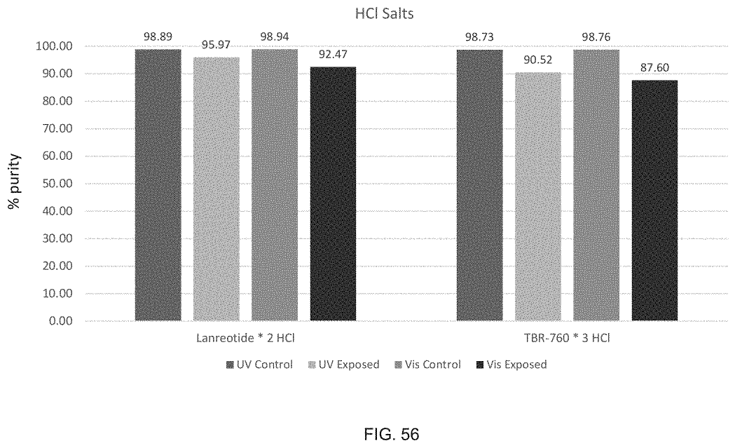

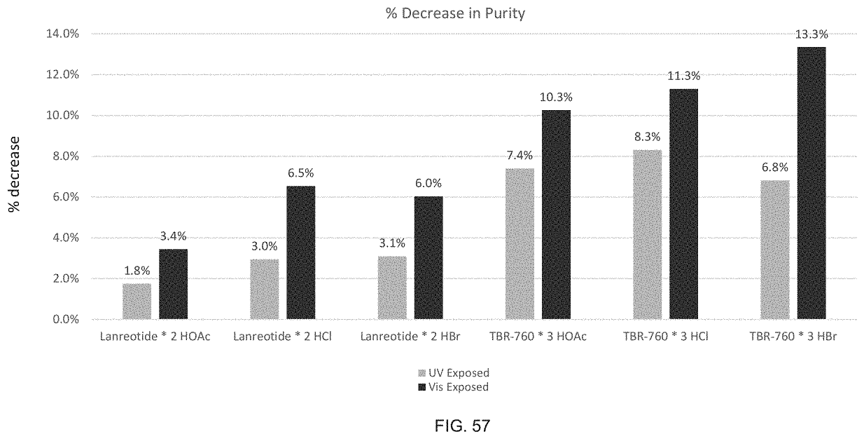

Following exposure to visible and UV light, all lanreotide and TBR-760 salts were analyzed by UPLC as described in Example 7. Results in FIGS. 54, 55, and 56 demonstrate that both lanreotide and TBR-760 degrade following exposure to visible and UV light, with exposure to visible light causing more degradation of each compound compared to exposure to UV light. TBR-760 bromide that is exposed to visible light shows a greater loss of purity than TBR-760 acetate or TBR-760 chloride under the same conditions (FIG. 57).

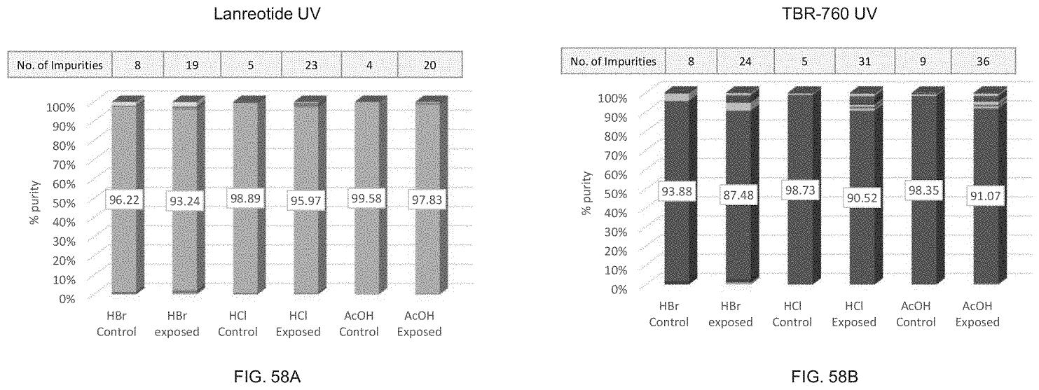

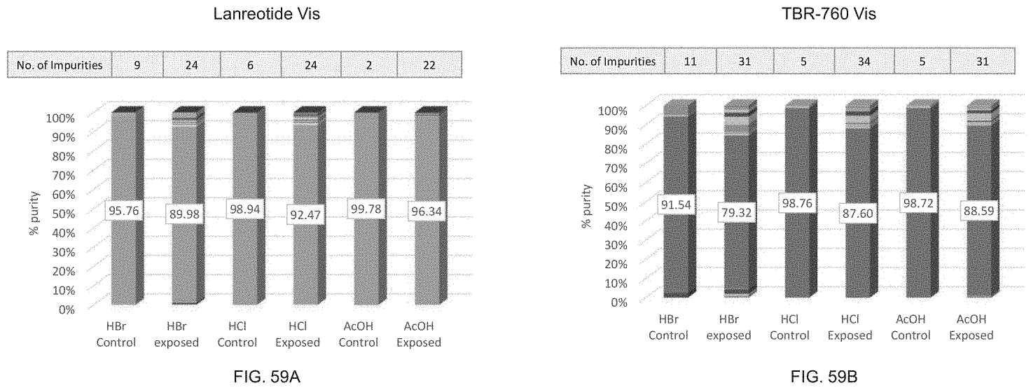

UPLC assays performed as described in Example 6 were further used to determine the number of degradants—and thus the number of impurities—in each sample. Results showing the purity assessment of each sample following photostability studies are shown in FIGS. 58A-58B (lanreotide and TBR-760 salts exposed to UV light) and FIGS. 59A-59B (lanreotide and TBR-760 salts exposed to visible light).

### 6. EQUIVALENTS AND INCORPORATION BY REFERENCE

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

What is claimed is:

1. The compound 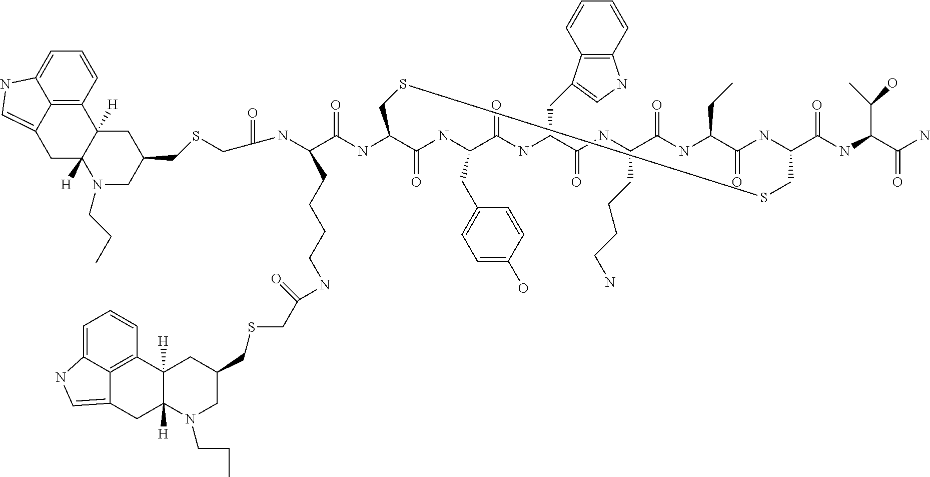 [SMILES: CCCN1C[C@H](CSCC(=O)NCCCC[C@@H](NC(=O)CSC[C@@H]2C[C@@H]3c4cccc5[nH]cc(c45)C[C@H]3N(CCC)C2)C(=O)N[C@H]2CSSC[C@@H](C(=O)N[C@H](C(N)=O)[C@@H](C)O)NC(=O)[C@H](CC)NC(=O)[C@H](CCCCN)NC(=O)[C@@H](Cc3c[nH]c4ccccc34)NC(=O)[C@H](Cc3ccc(O)cc3)NC2=O)C[C@@H]2c3cccc4[nH]cc(c34)C[C@H]21]
(TBR-760) hydrochloride salt.

2. The compound of claim 1, wherein the compound is a trihydrochloride salt.

3. The compound of claim 2, wherein less than 0.8% of the TBR-760 is degraded after 4 weeks at 60° C. and ambient relative humidity.

4. A storage stable pharmaceutical composition comprising the compound of claim 2.

5. The composition of claim 4, wherein less than 0.8% of the TBR-760 is degraded after 4 weeks at 60° C. and ambient relative humidity.

6. The composition of claim 4, wherein the composition is a lyophilate.

7. The composition of claim 6, wherein the water is in an amount of less than 10% (w/w).

8. The composition of claim 4, wherein the composition is a liquid solution.

9. A unit dosage form of the compound 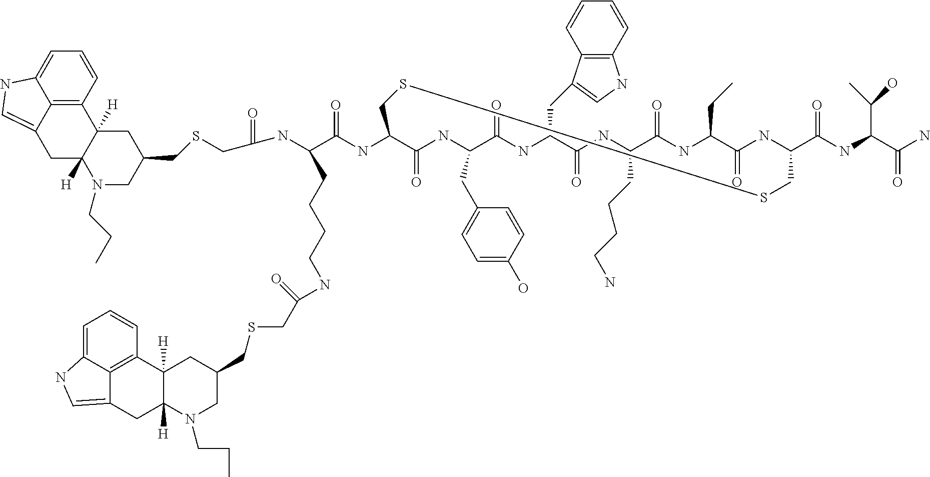 [SMILES: CCCN1C[C@H](CSCC(=O)NCCCC[C@@H](NC(=O)CSC[C@@H]2C[C@@H]3c4cccc5[nH]cc(c45)C[C@H]3N(CCC)C2)C(=O)N[C@H]2CSSC[C@@H](C(=O)N[C@H](C(N)=O)[C@@H](C)O)NC(=O)[C@H](CC)NC(=O)[C@H](CCCCN)NC(=O)[C@@H](Cc3c[nH]c4ccccc34)NC(=O)[C@H](Cc3ccc(O)cc3)NC2=O)C[C@@H]2c3cccc4[nH]cc(c34)C[C@H]21]
(TBR-760) hydrochloride salt in a pharmaceutical composition formulated for subcutaneous injection, wherein the unit dosage form is a pre-filled syringe or vial.

10. The unit dosage form of claim 9, wherein the unit dosage form is a pre-filled syringe.

11. The unit dosage form of claim 9, wherein the unit dosage form is a pre-filled vial.

12. The unit dosage form of claim 9, wherein the unit dosage form contains between 0.5 and 10 mg of TBR-760 hydrochloride.

13. The unit dosage form of claim 12, wherein the pre-filled syringe or vial contains a single dose of the pharmaceutical composition.

14. The unit dosage form of claim 10, wherein the pre-filled syringe is in an automated injection device.

15. A method of treating a subject having an endocrine disease or endocrine tumor, the method comprising:
administering to the subject an effective amount of a pharmaceutical composition comprising 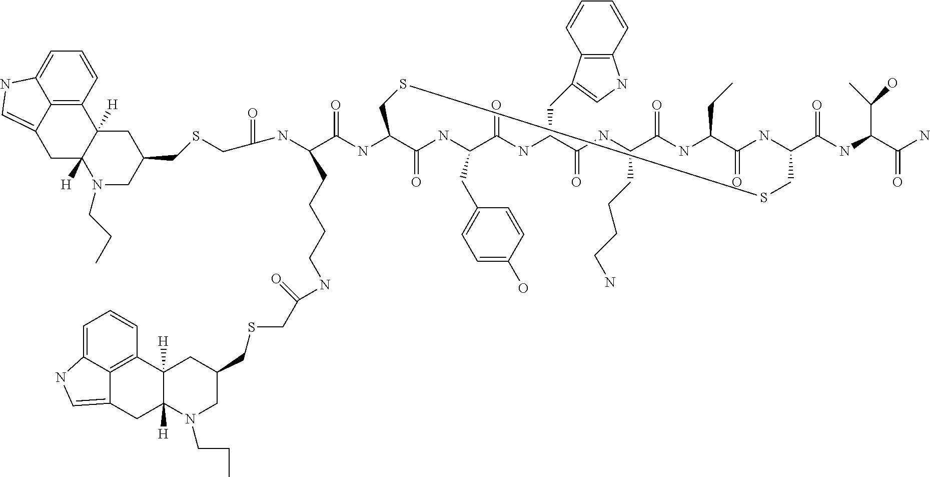 [SMILES: CCCN1C[C@H](CSCC(=O)NCCCC[C@@H](NC(=O)CSC[C@@H]2C[C@@H]3c4cccc5[nH]cc(c45)C[C@H]3N(CCC)C2)C(=O)N[C@H]2CSSC[C@@H](C(=O)N[C@H](C(N)=O)[C@@H](C)O)NC(=O)[C@H](CC)NC(=O)[C@H](CCCCN)NC(=O)[C@@H](Cc3c[nH]c4ccccc34)NC(=O)[C@H](Cc3ccc(O)cc3)NC2=O)C[C@@H]2c3cccc4[nH]cc(c34)C[C@H]21]
(TBR-760) hydrochloride salt.

16. The method of claim 15, wherein the subject has a nonfunctioning pituitary adenoma.
